(19) United States
(12) Patent Application Publication
(10) Pub. No.: US 2006/0211740 A1
(43) Pub. Date: Sep. 21, 2006

(54) Novel compounds

(76) Inventors: Emmanuel Hubert Demont, Welwyn (GB); Sally Redshaw, Welwyn (GB); Daryl Simon Walter, Welwyn (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/549,349

(22) PCT Filed: Mar. 11, 2004
(86) PCT No.: PCT/EP04/02644

§ 371 Date: Sep. 13, 2005

(30) Foreign Application Priority Data
Mar. 14, 2003 (GB) 0305918.5

(51) Int. Cl. A61K 31/44; A61K 31/18; C07D 213/78; C07C 311/08

(52) U.S. Cl. 514/352; 514/602; 546/310; 564/99

(57) ABSTRACT

The present invention relates to novel hydroxyethylamine compounds having Asp2 (β-secretase, BACE1 or Memapsin) inhibitory activity, processes for their preparation, to compositions containing them and to their use in the treatment of diseases characterised by elevated β-amyloid levels or β-amyloid deposits, particularly Alzheimer's disease.

The present invention relates to novel hydroxyethylamine compounds having Asp2 (β-secretase, BACE1 or Memapsin) inhibitory activity, processes for their preparation, to compositions containing them and to their use in the treatment of diseases characterised by elevated β-amyloid levels or β-amyloid deposits, particularly Alzheimer's disease.

Alzheimer's disease is a degenerative brain disorder in which extracellular deposition of β-amyloid (Aβ) in the form of senile plaques represents a key pathological hallmark of the disease (Selkoe, D. J. (2001) Physiological Reviews 81: 741-766). The presence of senile plaques is accompanied by a prominent inflammatory response and neuronal loss. Aβ exists in soluble and insoluble, fibrillar forms and a specific fibrillar form has been identified as the predominant neurotoxic species (Vassar, R. and Citron, M. (2000) Neuron 27: 419-422). In addition it has been reported that dementia correlates more closely with the levels of soluble amyloid rather than plaque burden (Naslund, J. et al. (2000) J. Am. Med. Assoc. 12:1571-1577; Younkin, S. (2001) Nat. Med. 1: 8-19). Aβ is known to be produced through the cleavage of the beta amyloid precursor protein (also known as APP) by an aspartyl protease enzyme known as Asp2 (also known as β-secretase, BACE1 or Memapsin) (De Strooper, B. and Konig, G. (1999) Nature 402: 471-472).

Therefore, it has been proposed that inhibition of the Asp2 enzyme would reduce the level of APP processing and consequently reduce the levels of Aβ peptides found within the brain. Therefore, it is also thought that inhibition of the Asp2 enzyme would be an effective therapeutic target in the treatment of Alzheimer's disease.

APP is cleaved by a variety of proteolytic enzymes (De Strooper, B. and Konig, G. (1999) Nature 402: 471-472). The key enzymes in the amyloidogenic pathway are Asp2 (β-secretase) and γ-secretase both of which are aspartic proteinases and cleavage of APP by these enzymes generates Aβ. The non-amyloidogenic, α-secretase pathway, which precludes Aβ formation, has been shown to be catalysed by a number of proteinases, the best candidate being ADAM10, a disintegrin and metalloproteinase. Asp1 has been claimed to show both α- and β-secretase activity in vitro. The pattern of expression of Asp1 and Asp2 are quite different, Asp2 is most highly expressed in the pancreas and brain while Asp1 expression occurs in many other peripheral tissues. The Asp2 knockout mouse indicates that lack of Asp2 abolished Aβ production and also shows that in this animal model endogenous Asp1 cannot substitute for the Asp2 deficiency (Luo, Y. et al. (2001) Nat Neurosci. 4: 231-232; Cal, H. et. al. (2001) Nat Neurosci. 4: 233-234; Roberds, S. L. et al. (2001) Hum. Mol. Genet 10: 1317-1324).

For an agent to be therapeutically useful in the treatment of Alzheimer's disease it is preferable that said agent is a potent inhibitor of the Asp2 enzyme, but should ideally also be selective for Asp2 over other enzymes of the aspartyl proteinase family, e.g Cathepsin D (Connor, G. E. (1998) Cathepsin D in Handbook of Proteolytic Enzymes, Barrett, A. J., Rawlings, N. D., & Woesner, J. F. (Eds) Academic Press London. pp828-836).

WO 01/70672, WO 02/02512, WO 02/02505 and WO 02/02506 (Elan Pharmaceuticals Inc.) describe a series of hydroxyethylamine compounds having β-secretase activity which are implicated to be useful in the treatment of Alzheimer's disease.

We have found a novel series of compounds which are potent inhibitors of the Asp2 enzyme, thereby indicating the potential for these compounds to be effective in the treatment of disease characterised by elevated β-amyloid levels or β-amyloid deposits, such as Alzheimer's disease.

Thus, according to a first aspect of the present invention we provide a compound of formula (I): 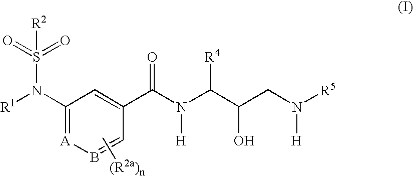
wherein
R¹ represents aryl or heteroaryl;
R² represents C₁₋₆ alkyl or C₃₋₈ cycloalkyl;
R^{2a} represents hydrogen, halogen, C₁₋₃ alkyl or C₁₋₃ alkoxy;
n represents 0, 1 or 2;
A represents —C(H)═, —C(R^{2b})═or —N═;
R^{2b} represents C₁₋₃ alkyl, C₂₋₄ alkenyl, halogen, C₁₋₃ alkoxy, amino, cyano or hydroxy;
B represents —C(R³)═or —N═;
R³ represents hydrogen, halogen, optionally substituted C₁₋₆ alkyl, C₂₋₆ alkenyl, aryl, heteroaryl, heterocyclyl, —C₁₋₆ alkyl-aryl, —C₁₋₆ alkyl-heteroaryl, —C₁₋₆ alkyl-heterocyclyl, —C₂₋₆ alkenyl-aryl, —C₂₋₆ alkenyl-heteroaryl, —C₂₋₆ alkenyl-heterocyclyl, C₃₋₈ cycloalkyl, —C₁₋₆ alkyl-C₃₋₈ cycloalkyl, cyano, azido, nitro, sulphoxide, —NR⁷R⁸, —NR⁹COR¹⁰, —NR¹¹SO₂R¹², —NR¹¹CO₂R¹², —OR³, —SO₂R¹⁴, —SR¹⁵, —C≡CR¹⁶, —C₀₋₆ alkyl-(CF₂)_{q}CF₃, —CONR¹⁷R¹⁸, COOR¹⁹, —C₁₋₆ alkyl-NR²⁰R²¹ or —C₁₋₆ alkyl-N₃, or R³ together with R^{2b} on adjacent carbon atoms may form a fused 5-7 membered saturated or partially saturated carbocyclic or heterocyclic ring optionally substituted by a C₁₋₆ alkyl group;
R⁴ represents optionally substituted C₁₋₆ alkyl, —C₁₋₆ alkyl-C₃₋₈ cycloalkyl, —C₁₋₆ alkyl-aryl, —C₁₋₆ alkyl-heteroaryl or —C₁₋₆ alkyl-heterocyclyl;
R⁵ represents hydrogen, optionally substituted C₁₋₁₀ alkyl, —C₃₋₈ cycloalkyl, —C₃₋₈ cycloalkenyl, aryl, heteroaryl, heterocyclyl, —C₁₋₆ alkyl-C₃₋₈ cycloalkyl, —C₃₋₈ cycloalkyl-aryl, -heterocyclyl-aryl, —C₁₋₆ alkyl-aryl-heteroaryl, —C(R^{a}R^{b})—CONH—C₁₋₆ alkyl, —C(R^{c}R^{d})—CONH—C₃₋₈ cycloalkyl, —C₂₋₆ alkyl-S—C₁₋₆ alkyl, —C₂₋₆ alkyl-NR^{e}R^{f}, —C(R^{g}R^{h})—C₁₋₆ alkyl, —C(RⁱR^{j})aryl, —C(R^{k}R^{l})—C₁₋₆ alkyl-aryl, —C(R^{m}Rⁿ)—C₁₋₆ alkyl-heteroaryl, —C(R^{o}R^{p})C₁₋₆ alkyl-heterocyclyl, —C₁₋₆ alkyl-O—C₁₋₆ alkyl-aryl, —C₁₋₆ alkyl-O—C₁₋₆ alkyl-heteroaryl or —C₁₋₆ alkyl-O—C₁₋₆ alkyl-heterocyclyl;
R⁷, R⁸, R⁹, R¹⁰, R¹¹, R¹³, R¹⁴, R¹⁵, R¹⁶, R¹⁷, R¹⁸, R¹⁹, R²⁰ and R²¹ independently represent hydrogen, C₁₋₆ alkyl, C₂₋₆ alkenyl, C₃₋₈ cycloalkyl, —CO—C₁₋₆ alkyl, aryl, heteroaryl, heterocyclyl, —C₁₋₆ alkyl-C₃₋₈ cycloalkyl, —C₁₋₆ alkyl-aryl, —C₁₋₆ alkyl-heteroaryl or —C₁₋₆ alkyl-heterocyclyl;
R^{a}, R^{c}, R^{e}, R^{f}, R^{g}, R^{h}, Rⁱ, R^{j}, R^{k}, R^{l}, R^{m}, Rⁿ, R^{o} and R^{p} independently represent hydrogen, C₁₋₆ alkyl or C₃₋₈ cycloalkyl;
R^{b} and R^{d} independently represent hydrogen, C₁₋₆ alkyl, C₃₋₈ cycloalkyl or —C₁₋₆ alkyl-SO₂—C₁₋₆ alkyl or R^{a} and R^{b}, R^{c} and R_{d}, R^{g} and R^{h}, Rⁱ and R^{j}, R^{k} and R^{l} and R^{m} and Rⁿ together with the carbon atom to which they are attached may form a C₃₋₈ cycloalkyl group;
R¹² represents C₁₋₆ alkyl or C₃₋₈ cycloalkyl;
q represents 0 to 3;
optional substituents for alkyl groups of R³, R⁴ and R⁵ include one or more (eg. 1, 2 or 3) halogen, C₁₋₆ alkoxy, amino, cyano or hydroxy groups;
and wherein said aryl, heteroaryl or heterocyclyl groups may be optionally substituted by one or more (eg. 1, 2 or 3) C₁₋₆ alkyl, halogen, —CF₃, —OCF₃, ═O, hydroxy, C₁₋₆ alkoxy, C₂₋₆ alkynyl, C₂₋₆ alkenyl, amino, cyano, nitro, —NR²²COR²³, —CONR²²R²³ —C₁₋₆ alkyl-NR²²R²³ (wherein R²² and R²³ independently represent hydrogen or C₁₋₆ alkyl), —C₁₋₆ alkyl-O—C₁₋₆ alkyl or —C₁₋₆ alkanoyl groups;
   or a pharmaceutically acceptable salt or solvate thereof.

In one particular aspect of the present invention, there is provided a compound of formula (I) as defined above wherein:
R² represents C₁₋₆ alkyl; and
R^{2a} represents hydrogen, halogen or C₁₋₃ alkyl; and
R³ represents hydrogen, halogen, optionally substituted C₁₋₆ alkyl, C₂₋₆ alkenyl, aryl, heteroaryl, heterocyclyl, —C₁₋₆ alkyl-aryl, —C₁₋₆ alkyl-heteroaryl, —C₁₋₆ alkyl-heterocyclyl, —C₂₋₆ alkenyl-aryl, —C₂₋₆ alkenyl-heteroaryl, —C₂₋₆ alkenyl-heterocyclyl, C₃₋₈ cycloalkyl, —C₁₋₆ alkyl-C₃₋₄ cycloalkyl, cyano, azido, nitro, sulphoxide, —NR⁷R⁸, —NR⁹COR¹⁰, —NR¹¹SO₂R¹², —OR¹³, —SO₂R¹⁴, —SR¹⁵, —C≡CR¹⁶, —C₀₋₆ alkyl-(CF₂)_{q}CF₃, —CONR¹⁷R¹⁸, COOR¹⁹, —C₁₋₆ alkyl-NR²⁰R²¹ or —C₁₋₆ alkyl-N₃, or R³ together with R^{2b} on adjacent carbon atoms may form a fused 5-7 membered saturated or partially saturated carbocyclic or heterocyclic ring; and
R⁵ represents hydrogen, optionally substituted C₁₋₁₀ alkyl, —C₃₋₈ cycloalkyl, —C₃₋₈ cycloalkenyl, aryl, heteroaryl, heterocyclyl, —C₁₋₆ alkyl-C₃₋₈ cycloalkyl, —C₃₋₈ cycloalkyl-aryl, -heterocyclyl-aryl, —C₁₋₆ alkyl-aryl-heteroaryl, —C(R^{a}R^{b})—CONH—C₁₋₆ alkyl, —C(R^{c}R^{d})CONH—C₃₋₈ cycloalkyl, —C₁₋₆ alkyl-S—C₁₋₆ alkyl, —C₁₋₆ alkyl-NR^{e}R^{f}, —C(R^{g}R^{h})—C₁₋₆ alkyl, —C(RⁱR^{j})-aryl, —C(R^{k}R^{l})—C₁₋₆ alkyl-aryl, —C(R^{m}Rⁿ)—C₁₋₆ alkyl-heteroaryl, —C(R^{o}R^{p})—C₁₋₆ alkyl-heterocyclyl, —C₁₋₆ alkyl-O—C₁₋₆ alkyl-aryl, —C₁₋₆ alkyl-O—C₁₋₆ alkyl-heteroaryl or —C₁₋₆ alkyl-O—C₁₋₆ alkyl-heterocyclyl; and
R¹¹, R^{a}, R^{c}, R^{e}, R^{f}, R^{g}, R^{h}, Rⁱ, R^{j}, R^{k}, R^{l}, R^{m}, Rⁿ, R^{o} and R^{p} independently represent hydrogen, C₁₋₆ alkyl or C₃₋₈ cycloalkyl; and
q represents 1 to 3.

References to alkyl include references to both straight chain and branched chain aliphatic isomers of the corresponding alkyl. It will be appreciated that references to alkenyl shall be interpreted similarly.

References to C₃₋₈ cycloalkyl include references to all alicyclic (including branched) isomers of the corresponding alkyl.

References to ‘aryl’ include references to monocyclic carbocyclic aromatic rings (eg. phenyl) and bicyclic carbocyclic aromatic rings (e.g. naphthyl) or carbocyclic benzofused rings (eg. C₃₋₈ cycloalkyl fused to a phenyl ring).

References to ‘heteroaryl’ include references to mono- and bicyclic heterocyclic aromatic rings containing 1-4 hetero atoms selected from nitrogen, oxygen and sulphur. Examples of monocyclic heterocyclic aromatic rings include e.g. thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl, pyridyl, tetrazolyl and the like. Examples of bicyclic heterocyclic aromatic rings include eg. quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like.

References to ‘heterocyclyl’ include references to a 5-7 membered non-aromatic monocyclic ring containing 1 to 3 heteroatoms selected from nitrogen, sulphur or oxygen. Examples of heterocyclic non-aromatic rings include e.g. morpholinyl, piperidinyl, piperazinyl, thiomorpholinyl, oxathianyl, dithianyl, dioxanyl, pyrrolidinyl, dioxolanyl, oxathiolanyl, imidazolidinyl, pyrazolidinyl and the like.

Preferably, R¹ represents aryl (eg. phenyl or naphthyl) or heteroaryl (eg. pyridyl) optionally substituted by one or more halogen (eg. fluorine or chlorine), cyano, —CF₃, C₁₋₆ alkoxy (eg. methoxy) or —CONR²²R²³ (eg.—CONH₂) groups.

More preferably, R¹ represents aryl (eg. phenyl or naphthyl) or heteroaryl (eg. pyridyl) optionally substituted by one or more halogen (eg. fluorine or chlorine) atoms.

Most preferably, R¹ represents aryl, eg. unsubstituted phenyl or phenyl substituted by a halogen (eg. fluorine or chlorine), cyano, or C₁₋₆ alkoxy (eg. methoxy) group.

Preferably, R² represents methyl, ethyl, i-propyl or butyl, more preferably methyl.

Preferably, n is 0 or 1, more preferably 1.

When n represents 1, R^{2a} is preferably C₁₋₃ alkyl (eg. methyl) or halogen (eg. fluorine), more preferably halogen (eg. fluorine).

When n represents 1, R^{2a} is preferably at the para position of the carbocyclic/heterocyclic ring with respect to the group B.

When A represents —C(R^{2b})═, R^{2b} is preferably:
halogen (eg. chlorine or fluorine); or
C₁₋₃alkyl (eg. methyl).

When A represents —C(R^{2b})═, R^{2b} is more preferably fluorine.

Preferably, A represents —C(H)═ or —N═, more preferably —C(H)═.

Preferably, B represents —C(R³)═.

Preferably, R³ represents:
halogen (eg. bromine);
C₁₋₆ alkyl (eg. ethyl, propyl, i-propyl or t-butyl);
C₂₋₆ alkenyl (eg. CH═CH₂, —CH═C(CH₃)₂, —CH(CH₃)═CH₂ or —CH═CH—CH₃);
C₃₋₈ cycloalkyl (eg. cyclopentyl or cyclohexyl);
cyano;

aryl (eg. phenyl) optionally substituted by one or more C₁₋₆ alkyl (eg. methyl) groups;
heterocyclyl (eg. pyrrolidinyl or isothiazolidinyl) optionally substituted by one or two oxo groups (eg. 2-oxopyrrolidinyl or 1,1-dioxo-isothiazolidinyl);
—NR⁷R⁸;
—OR¹³;
—SO₂R¹⁴;
—SR¹⁵;
—C≡CR¹⁶; or
—CONR¹⁷R¹⁸.

Also preferably, R³ together with R^{2b} on adjacent carbon atoms forms a partially saturated heterocyclic (eg. pyrroline) group optionally substituted by a C₁₋₆ alkyl group (eg. ethyl).

More preferably, R³ represents:
halogen (eg. bromine);
C₁₋₆ alkyl (eg. i-propyl);
C₂₋₆ alkenyl (eg.—CH═CH₂, —CH═C(CH₃)₂, —CH(CH₃)═CH₂ or —CH═CH—CH₃);
C₃₋₈ cycloalkyl (eg. cyclopentyl);
—NR⁷R⁸;
—OR¹³;
—SR¹⁵; or
heterocyclyl (eg. pyrrolidinyl) substituted by an oxo group (eg. 2-oxopyrrolidinyl).

Preferably, R⁷ and R⁸ independently represent:
hydrogen;
C₁₋₆ alkyl (eg. methyl, ethyl, propyl, butyl, pentyl, i-propyl, i-butyl, ethylpropyl, dimethylpropyl or methylbutyl);
C₃₋₈ cycloalkyl (eg. cyclopentyl or cyclohexyl);
aryl (eg. phenyl);
—C₁₋₆ alkyl-C₃₋₈ cycloalkyl (eg.—CH₂-cyclopropyl);
—C₁₋₆ alkyl-aryl (eg. —CH₂-phenyl or —(CH₂)₂-phenyl); or
—CO—C₁₋₆ alkyl (eg.—COCH₃).

More preferably, R⁷ and R⁸ independently represent hydrogen, C₁₋₆ alkyl (eg. methyl, ethyl or isopropyl) or —C₁₋₆ alkyl-aryl (eg.—CH₂-phenyl), especially hydrogen or C₁₋₆ alkyl.

Most preferably, R⁷ represents hydrogen and R⁸ represents C₁₋₆ alkyl (eg. ethyl or isopropyl).

Preferably, R¹³ represents C₁₋₆ alkyl (eg. ethyl or isopropyl).

Preferably, R¹⁴ and R¹⁵ independently represent C₁₋₆ alkyl (eg. methyl or ethyl).

More preferably, R¹⁵ represents ethyl.

Preferably, R¹⁶ represents hydrogen or C₁₋₆ alkyl (eg. methyl).

Preferably, R¹⁷ and R¹⁸ independently represent C₁₋₆ alkyl (eg. propyl).

Preferably, R⁴ represents —C₁₋₆ alkyl-aryl (eg. benzyl) optionally substituted by one or two halogen atoms (eg. fluorine). More preferably, R⁴ represents unsubstituted benzyl.

Preferably, R⁵ represents:
—C₁₋₁₀ alkyl (eg. 1,5-dimethylhexyl or 1,1,5-trimethylhexyl);
—C₃₋₈ cycloalkyl (eg. cyclopropyl or cyclohexyl);
—C(RⁱR^{j})-aryl (eg. benzyl or 1-phenyl-1-methylethyl) optionally substituted (eg. substituted at the 3 and 5 positions) by one or more halogen, cyano, —OCF₃, —CF₃, C₁₋₆ alkyl or C₁₋₆ alkoxy (eg. methoxy) groups;
—C(R_{c}R_{d})—CONH—C₃₋₈ cycloalkyl (eg. C(R^{c}R^{d})—CONH-cyclohexyl); or
—C₃₋₈ cycloalkyl-aryl.

More preferably, R⁵ represents:
—C(RⁱR^{j})-aryl (eg. benzyl) optionally substituted by an —OCF₃ or —CF₃ group; or
—C(R^{c}R^{d})—CONH—C₃₋₈ cycloalkyl (eg. C(R^{c}R^{d})—CONH-cyclohexyl).

Preferably, R^{c} and R^{d} independently represent hydrogen or methyl, more preferably R^{c} represents hydrogen and R^{d} represents methyl.

Preferably, Rⁱ and R^{j} independently represent hydrogen or C₁₋₆ alkyl (eg. methyl) or together with the carbon atom to which they are attached form a C₃₋₈ cycloalkyl group.

Preferred compounds according to the invention includes examples E1-E90 as shown below, or a pharmaceutically acceptable salt thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic or organic acids e.g. hydrochlorides, hydrobromides, sulphates, phosphates, acetates, benzoates, citrates, nitrates, succinates, lactates, tartrates, fumarates, maleates, 1-hydroxy-2-naphthoates, palmoates, methanesulphonates, p-toluenesulphonates, naphthalenesulphonates, formates or trifluoroacetates. The present invention includes within its scope all possible stoichiometric and non-stoichiometric forms.

The compounds of formula (I) may be prepared in crystalline or non-crystalline form, and, if crystalline, may optionally be solvated, eg. as the hydrate. This invention includes within its scope stoichiometric solvates (eg. hydrates) as well as compounds containing variable amounts of solvent (eg. water).

Certain compounds of formula (I) are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof. Preferably, compounds of formula (I) are in the form of a single enantiomer of formula (Ia):

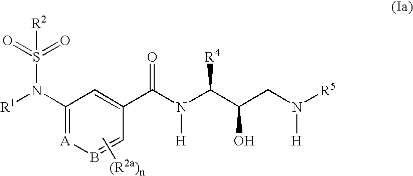

The compounds of formula (I) and salts and solvates thereof may be prepared by the methodology described hereinafter, constituting a further aspect of this invention.

A process according to the invention for preparing a compound of formula (I) which comprises:

(a) reacting a compound of formula (11) 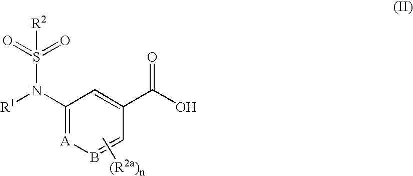
or an activated and/or optionally protected derivative thereof wherein R¹, R², R^{2a}, n, A and B are as defined above, with a compound of formula (III) 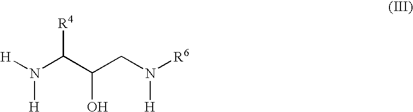
wherein R⁴ and R⁵ are as defined above; or

(b) preparing a compound of formula (I) which comprises reductive alkylation of a compound of formula (IV)

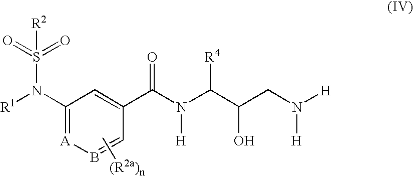

wherein R¹, R², R^{2a}, n, A, B and R⁴ are as defined above, with an appropriate aldehyde or ketone; or

(c) deprotecting a compound of formula (I) which is protected; and optionally thereafter

(d) interconversion of compounds of formula (I) to other compounds of formula (I).

Where the compound of formula (II) is an activated derivative, (eg. by activation of a carboxylic acid to an acid chloride, mixed anhydride, active ester e.g. mesylate or tosylate, O-acyl-isourea or other species), process (a) typically comprises treatment of said activated derivative with an amine (Ogliaruso, M. A.; Wolfe, J. F. in The Chemistry of Functional Groups (Ed. Patai, S.) Suppl. B: The Chemistry of Acid Derivatives, Pt. 1 (John Wiley and Sons, 1979), pp 442-8; Beckwith, A. L. J. in The Chemistry of Functional Groups (Ed. Patai, S.) Suppl. B: The Chemistry of Amides (Ed. Zabricky, J.) (John Wiley and Sons, 1970), p 73 ff. The acid of formula (II) and amine are preferably reacted in the presence of an activating agents such as 1-(dimethylaminopropyl)-3-ethylcarbodiimide hydrochloride (EDC) and 1-hydroxybenzotriazole (HOBT), or O-(7-azabenzotriazol-1-yl)-N,N,N′,N′-tetramethyluronium hexafluorophosphate (HATU)

Where the compound of formula (II) is a carboxylic acid, process (a) typically comprises the use of water soluble carbodiimide, HOBT and a suitable base such as tertiary alkylamine or pyridine in a suitable solvent such as DMF and at a suitable temperature, eg. between 0° C. and room temperature.

Process (b) typically comprises the use of sodium borohydride triacetate in the presence of a suitable solvent, such as ethanol, dichloromethane and 1,2-dichloroethane and at a suitable temperature, e.g. between 0° C. and room temperature.

In process (c), examples of protecting groups and the means for their removal can be found in T. W. Greene and P. G. M. Wuts ‘Protective Groups in Organic Synthesis’ (J. Wiley and Sons, 3rd Ed. 1999). Suitable amine protecting groups include aryl sulphonyl (e.g. tosyl), acyl (e.g. acetyl), carbamoyl (e.g. benzyloxycarbonyl or t-butoxycarbonyl) and arylalkyl (e.g. benzyl), which may be removed by hydrolysis or hydrogenolysis as appropriate. Other suitable amine protecting groups include trifluoroacetyl (—COCF₃) which may be removed by base catalysed hydrolysis. Suitable hydroxy protecting groups would be silyl based groups such as t-butyldimethylsilyl, which may be removed using standard methods, for example use of an acid such as trifluoroacetic or hydrochloric acid or a fluoride source such as tetra n-butylammonium fluoride.

Process (d) may be performed using conventional interconversion procedures such as epimerisation, oxidation, reduction, alkylation, aromatic substitution, ester hydrolysis, amide bond formation or removal and sulphonylation. An example of such an interconversion reaction may include interconversion of a compound of formula (I) wherein B represents —C(R³)═ and R³ represents a C₂₋₆ alkenyl containing group to a corresponding compound of formula (I) wherein R³ represents a C₂₋₆ alkyl containing group, using standard hydrogenation or reductive conditions. A further example of such an interconversion reaction may include interconversion of a compound of formula (I) wherein R³ represents —C₁₋₆ alkyl-N₃ to a corresponding compound of formula (I) wherein R³ represents —C₁₋₆ alkyl-NH₂, using standard hydrogenation or reductive conditions. A yet further example of such an interconversion reaction may include interconversion of a compound of formula (I) wherein R³ represents a nitro group to a corresponding compound of formula (I) wherein R³ represents NH₂, using standard hydrogenation or reductive conditions. A yet further example of such an interconversion reaction may include interconversion of a compound of formula (I) wherein R³ represents a halogen atom to a corresponding compound of formula (I) wherein R³ represents a C₂₋₆ alkenyl group, using standard Suzuki coupling conditions.

Compounds of formula (II) and/or activated and optionally protected derivatives thereof may be prepared in accordance with the following process: 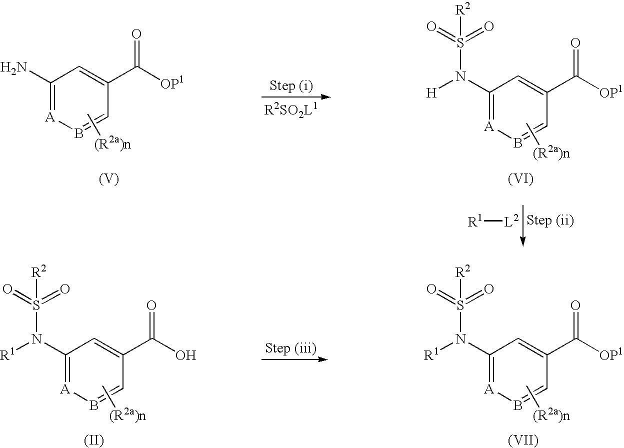
wherein R^{2a}, n, A, B, R¹ and R² are as defined above, P¹ represents a suitable group such as C₁₋₆ alkyl, L¹ represents a suitable leaving group such as a halogen atom (eg. Iodine, chlorine or bromine) and L² represents a suitable group such as boronic acid or a boronic ester.

Step (i) typically comprises the use of a suitable solvent such as dichloromethane and suitable bases such as pyridine and dimethylaminopyridine at a suitable temperature, such as room temperature.

Step (ii) typically comprises the use of copper (II) acetate in the presence of a suitable solvent such as dichloromethane and a suitable base such as triethylamine at a suitable temperature, such as room temperature (Chan et al., (1998) Tetrahedron Letters 39, 2933-2936).

Step (iii) typically comprises a standard procedure for conversion of a carboxylic ester to an acid, such as the use of an appropriate hydroxide salt like lithium or sodium salt in an appropriate solvent such as methanol at an appropriate temperature such as room temperature. In the case of a tert-butyl ester this conversion can be achieved by the use of an appropriate acid such as trifluoroacetic acid in an appropriate solvent such as dichloromethane at an appropriate temperature such as O° C. Activated derivatives of compounds of formula (II) may then be prepared as described in process (a) above.

Compounds of formula (II) or activated and optionally protected derivatives thereof may also be prepared in accordance with the following process: 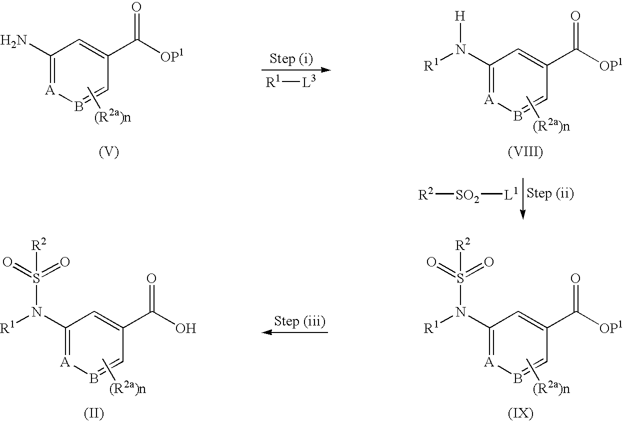
wherein R^{2a}, n, A, B, R¹, R², P¹ and L¹ are as defined above and L³ represents a suitable leaving group such as a halogen atom (eg. bromine, chlorine or iodine), OSO₂(CF₂)₀₋₇CF₃.

Step (i) typically comprises the use of caesium carbonate, 4,5-bis(diphenylphosphino) 9,9-dimethylxanthene and a suitable catalyst such as tris(dibenzylideneacetone)dipalladium(0) in the presence of a suitable solvent (eg. toluene) at a suitable temperature, eg. 100° C.

Step (ii) typically comprises the use of lithium diisopropylamide in the presence of a suitable solvent such as tetrahydrofuran at a suitable temperature, eg. heating from −78° C. to room temperature.

Step (iii) typically comprises a standard procedure for conversion of a carboxylic ester to an acid, such as the use of an appropriate hydroxide salt such as lithium or sodium salt in an appropriate solvent such as methanol at an appropriate temperature such as room temperature. In the case of a tert-butyl ester this conversion can be achieved by the use of an appropriate acid such as trifluoroacetic acid in an appropriate solvent such as dichloromethane at an appropriate temperature such as 0° C. Activated derivatives of compounds of formula (II) may then be prepared as described in process (a) above.

Compounds of formula (III) may be prepared in accordance with the following process: 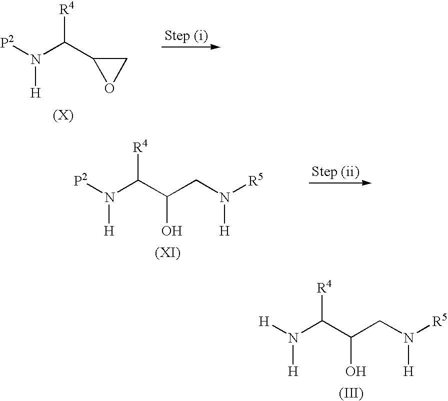
wherein R⁴ and R⁵ are as defined above and P² represents a suitable amine protecting group, such as t-butoxycarbonyl.

Step (i) typically comprises the reaction of a compound of formula (X) with a compound of formula NH₂R⁵ in the presence of a suitable solvent, e.g. ethanol at a suitable temperature, e.g. reflux.

Step (ii) typically comprises the use of suitable deprotection reactions as described above for process (c), eg. when P² represents t-butoxycarbonyl, deprotection typically comprises the use of trifluoroacetic acid in the presence of a suitable solvent, such as dichloromethane at a suitable temperature, e.g. between 0° C. and room temperature.

Compounds of formula (IV) may be prepared in accordance with the following process: 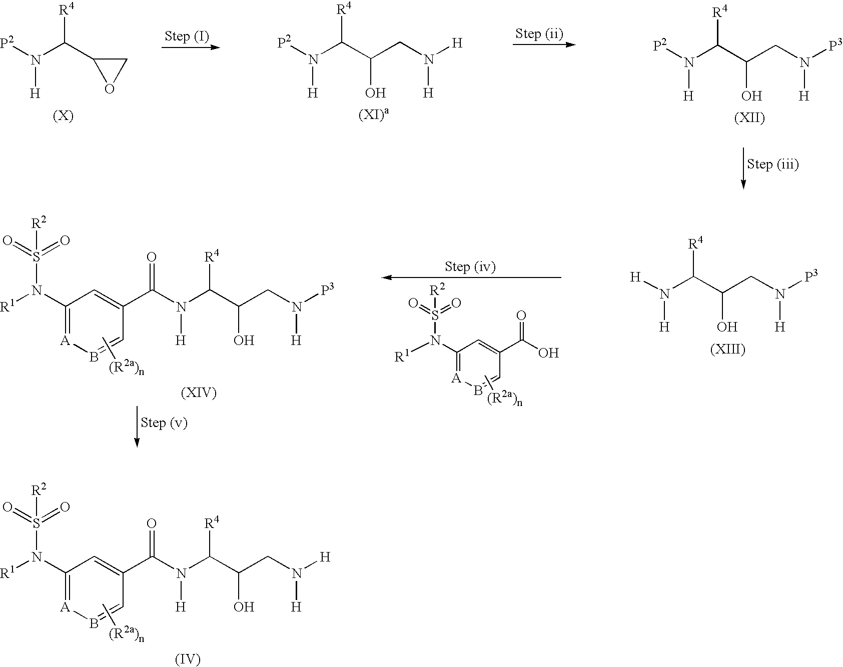
wherein R¹, R², R^{2a}, n, A, B, R⁴ and P² are as defined above and P³ represents a suitable amine protecting group different to P², such as —COOCH₂-phenyl.

Step (i) typically comprises the reaction of a compound of formula (X) in aqueous ammonia in the presence of a suitable solvent, e.g. ethanol at a suitable temperature, e.g. reflux.

When P³ represents —COOCH₂-phenyl, step (ii) typically comprises the use of ClCOOCH₂-phenyl in the presence of a suitable base, e.g. triethylamine, a suitable solvent, e.g. dimethylformamide at a suitable temperature, e.g. between 0° C. and room temperature.

Step (iii) typically comprises the use of suitable deprotection reactions as described above for process (c), eg. when P² represents t-butoxycarbonyl, deprotection typically comprises the use of trifluoroacetic acid in the presence of a suitable solvent, such as dichloromethane at a suitable temperature, e.g. between 0° C. and room temperature.

Step (iv) typically comprises reacting a compound of formula (XIII) with a compound of formula (II) in the presence of water soluble carbodiimide and HOBT.

Step (v) typically comprises the use of suitable deprotection reactions as described above for process (c), eg. when P³ represents —COOCH₂-phenyl, deprotection typically comprises the use of a suitable catalyst, eg. palladium in the presence of a suitable solvent, e.g. water and ethanol and in the presence of a suitable hydrogen source, e.g. ammonium formate at a suitable temperature, eg. 60° C.

Compounds of formula (V) are either commercially available or may be prepared from commercially available compounds using standard procedures.

Compounds of formula (X) are either known or may be prepared in accordance with known procedures.

As a further aspect of the invention there is thus provided a compound of formula (I) or a pharmaceutically acceptable salt or solvate thereof for use as a pharmaceutical, particularly in the treatment of patients with diseases characterised by elevated β-amyloid levels or β-amyloid deposits.

According to another aspect of the invention, there is provided the use of a compound of formula (I) or a physiologically acceptable salt or solvate thereof for the manufacture of a medicament for the treatment of patients with diseases characterised by elevated β-amyloid levels or β-amyloid deposits.

In a further or alternative aspect there is provided a method for the treatment of a human or animal subject with diseases characterised by elevated β-amyloid levels or β-amyloid deposits, which method comprises administering to said human or animal subject an effective amount of a compound of formula (I) or a physiologically acceptable salt or solvate thereof.

As a further aspect of the invention there is thus provided a pharmaceutical composition comprising a compound of formula (I) or a pharmaceutically acceptable salt or solvate thereof for use in the treatment of diseases characterised by elevated β-amyloid levels or β-amyloid deposits.

It will be appreciated by those skilled in the art that reference herein to treatment extends to prophylaxis as well as the treatment of diseases characterised by elevated β-amyloid levels or β-amyloid deposits.

The compounds according to the invention may be formulated for administration in any convenient way, and the invention therefore also includes within its scope pharmaceutical compositions for use in the therapy of diseases characterised by elevated β-amyloid levels or β-amyloid deposits, comprising a compound of formula (I) or a physiologically acceptable salt or solvate thereof together, if desirable, with one or more physiologically acceptable diluents or carriers.

It will be appreciated that diseases characterised by elevated β-amyloid levels or β-amyloid deposits include Alzheimer's disease, mild cognitive impairment, Down's syndrome, hereditary cerebral haemorrhage with β-amyloidosis of the Dutch type, cerebral β-amyloid angiopathy and various types of degenerative dementias, such as those associated with Parkinson's disease, progressive supranuclear palsy, cortical basal degeneration and diffuse Lewis body type of Alzheimer's disease.

Most preferably, the disease characterised by elevated β-amyloid levels or β-amyloid deposits is Alzheimer's disease.

There is also provided a process for preparing such a pharmaceutical formulation which comprises mixing the ingredients.

Compounds of formula (I) may be used in combination with other therapeutic agents. Suitable examples of such other therapeutic agents may be acetylcholine esterase inhibitors (such as tetrahydroaminoacridine, donepezil hydrochloride and rivastigmine), gamma secretase inhibitors, anti-inflammatory agents (such as cyclooxygenase II inhibitors), antioxidants (such as Vitamin E and ginkolidesor), statins or p-glycoprotein (P-gp) inhibitors (such as cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102 and 918).

When the compounds are used in combination with other therapeutic agents, the compounds may be administered either sequentially or simultaneously by any convenient route.

The compounds according to the invention may, for example, be formulated for oral, inhaled, intranasal, buccal, enteral, parenteral, topical, sublingual, intrathecal or rectal administration, preferably for oral administration.

Tablets and capsules for oral administration may contain conventional excipients such as binding agents, for example syrup, acacia, gelatin, sorbitol, tragacanth, mucilage of starch, cellulose or polyvinyl pyrrolidone; fillers, for example, lactose, microcrystalline cellulose, sugar, maize- starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavouring, colouring and/or sweetening agents (e.g. mannitol) as appropriate.

For buccal administration the compositions may take the form of tablets or lozenges formulated in conventional manner.

The compounds may also be formulated as suppositories, e.g. containing conventional suppository bases such as cocoa buffer or other glycerides.

The compounds according to the invention may also be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or tonicity adjusting agents. Alternatively, the active ingredient may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

When the compounds of the invention are administered topically they may be presented as a cream, ointment or patch.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 3000 mg; and such unit doses may be administered more than once a day, for example one, two, three or four times per day (preferably once or twice); and such therapy may extend for a number of weeks, months or years.

All publications, including but not limited to patents and patent applications, cited in this specification are herein incorporated by reference as if each individual publication were specifically and individually indicated to be incorporated by reference herein as though fully set forth.

### EXAMPLES

Preparation of Intermediates

Description F1

((S)-1-Cyclohexylcarbamoyl-ethyl)-carbamic acid tert-butyl ester (F1)

(S)-2-tert-Butoxycarbonylamino-propionic acid (1.5 g, 8.0 mmol, 1 equiv), 1-(3-dimethylaminopropyl)-3-ethyl-carbodiimide hydrochloride (1.84 g, 9.6 mmol, 1.2 equiv), 1-hydroxybenzotriazole hydrate (1.47 g, 9.6 mmol, 1.2 equiv), 4-ethylmorpholine (1.76 g, 16 mmol, 2 equiv) and cyclohexylamine (1.1 ml, 9.6 mmol, 1.2 equiv) in CH₂Cl₂ (10 ml) were stirred at room temperature for 16 h. The solution was concentrated in vacuo and the residue dissolved in AcOEt. The organic phase was washed with 2N aqueous HCl solution, saturated aqueous NaHCO₃ solution and brine, dried over MgSO₄ and concentrated in vacuo to give ((S)-1-cyclohexylcarbamoyl-ethyl)-carbamic acid tert-butyl ester (F1) (2.2 g, 98%) as a colourless oil.

Description F2

2-(3-Methoxy-phenyl)-2-methyl-propionic acid ethyl ester (F2)

To a solution of (3-methoxy-phenyl)-acetic acid ethyl ester (19.72 g, 0, 101 m, 1 equiv) in THF (200 ml) was added NaH (60% in mineral oil, 8.8 g, 0.222 mol, 2.2 equiv) then iodomethane (26 ml, 0.4 mol, 4 equiv). The resulting mixture was stirred at room temperature for 16 h then partitioned between AcOEt and saturated NaHCO₃ aqueous solution. The two layers were separated and the organic phase washed with brine, dried over MgSO₄ and concentrated in vacuo to give 2-(3-methoxy-phenyl)-2-methyl-propionic acid ethyl ester (F2) (20.85 g, 98%) as an orange oil.

Description F3

2-(3-Methoxy-phenyl)-2-methyl-propionic acid (F3)

To a solution of 2-(3-methoxy-phenyl)-2-methyl-propionic acid ethyl ester (F2) (20.95 g, 94 mmol, 1 equiv) in EtOH (200 ml) was added 2N NaOH aqueous solution (90 ml, 180 mmol, 1.9 equiv) and the resulting mixture was stirred at 70° C. for 16 h then cooled to room temperature. Most of EtOH was removed in vacuo and the residue extracted with AcOEt then acidified to pH 1. The aqueous phase was then extracted with AcOEt and the organic phase dried over MgSO₄ and concentrated in vacuo to give 2-(3-methoxy-phenyl)-2-methyl-propionic acid (F3) (15 g, 82%) as a yellow oil.

Description F4

[1-(3-Methoxy-phenyl)-1-methyl-ethyl]-carbamic acid benzyl ester (F4)

To a solution of 2-(3-methoxy-phenyl)-2-methyl-propionic acid (F3) (1 g, 5.15 mmol, 1 equiv) in toluene (20 ml) at room temperature was added NEt₃ (1.07 ml, 7.72 mmol, 1.5 equiv) and then diphenylphosphoryl azide (2.2 ml, 10.3 mmol, 2 equiv). The resulting mixture was then heated at 80° C. for 2 h then benzyl alcohol (1.61 ml, 15.45 mmol, 3 equiv) was added and the solution heated for a further 2 h, cooled to room temperature and partitioned between EtOAc and saturated NaHCO₃ aqueous solution. The two layers were separated and the aqueous phase dried over MgSO₄ and concentrated in vacuo. Purification of the residue by flash chromatography on silica gel (isohexane/AcOEt: 9/1) gave [1-(3-methoxy-phenyl)-1-methyl-ethyl]-carbamic acid benzyl ester (F4) (1 g, 65%) as a yellow gum.

Description F5

1-(3-Methoxy-phenyl)-1-methyl-ethylamine (F5)

A flask was charged with [1-(3-methoxy-phenyl)-1-methyl-ethyl]-carbamic acid benzyl ester (F4) (1 g, 3.34 mmol, 1 equiv), 10% palladium on charcoal (50% wet, 100 mg, 10% w/w), NH₄COOH (2.1 g, 33 mmol, 10 equiv), EtOH (40 ml) and H₂O (8 ml). The resulting mixture was stirred at 80° C. for 2 h, cooled to room temperature and the catalyst was filtered off using a pad of celite. Most of the EtOH was removed in vacuo and the residue was diluted with 1N HCl aqueous solution. The aqueous phase was extracted with AcOEt then basified to pH 13 and extracted twice with AcOEt. The combined organic layers were dried over MgSO₄ and concentrated in vacuo to give 1-(3-methoxy-phenyl)-1-methyl-ethylamine (F5) (290 mg, 53%) as a yellow gum.

Description F6

(S)-2-Amino-N-cyclohexyl-propionamide (F6)

((S>1-Cyclohexylcarbamoyl-ethyl)carbamic acid tert-butyl ester (F1) (2.32 g, 8.6 mmol, 1 equiv) was dissolved in 4M HCl in dioxan (40 ml) and the solution was stirred for 1 h at room temperature then concentrated in vacuo. The residue was triturated with Et₂O to give (S)-2-amino-N-cyclohexyl-propionamide hydrochloride salt (F6) (2.0 g, 95%) as a white solid.

Description F7

5-Nitro-isophthalamic acid methyl ester (F7)

A suspension of 5-nitro-isophthalic acid monomethyl ester (10 g, 44 mmol, 1 equiv) in CH₂Cl₂ (250 ml) was treated with (COCl)₂ (4.36 ml, 50 mmol, 1.1 equiv) followed by a few drops of DMF. The resulting mixture was stirred for 30 min at 35° C. then cooled to 0° C. 32% aqueous ammonia (10 ml, excess) was slowly added and the resulting mixture was stirred for 5 min. The precipitate formed was filtered to give 5-nitro-isophthalamic acid methyl ester (F7) (9 g, 90%) as a white solid.

Description F8

3-Cyano-5-nitro-benzoic acid methyl ester (F8)

A suspension of 5-nitro-isophthalamic acid methyl ester (F7) (500 mg, 2.23 mmol, 1 equiv) in CH₂Cl₂ (100 ml) was treated with NEt₃ (1.2 g, 12.0 mmol, 5.4 equiv) and trifluoroacetic anhydride (1.4 mg, 6.7 mmol, 3 equiv). The resulting mixture was stirred for 3 h at room temperature then washed with 2N aqueous HCl solution (50 ml), saturated aqueous NaHCO₃ solution (50 ml), dried over MgSO₄. and concentrated in vacuo. The residue was triturated with AcOEt and iso-hexane to give 3-cyano-5-nitro-benzoic acid methyl ester (F8) (250 mg, 54%) as a white solid.

Description F9

3-Amino-5-cyano-benzoic acid methyl ester (F9)

To a solution of 3-cyano-5-nitro-benzoic acid methyl ester (F8) (700 mg, 3.39 mmol, 1 equiv) in EtOH (50 ml) was added SnCl₂ (3.2 g, 17 mmol, 5 equiv). The resulting mixture was stirred at reflux for 2 h, cooled to room temperature and concentrated in vacuo. The residue was partitioned between ice-cold AcOEt and H₂O. The aqueous phase was basified with 2N aqueous NaOH solution until a white precipitate appeared, then slowly with 12.5N aqueous NaOH solution until this precipitate disappeared. The temperature was kept below 10° C. during this addition. The two layers were separated and the aqueous phase extracted with AcOEt. The combined organic layers were washed with brine, dried over MgSO₄ and concentrated in vacuo to give 3-amino-5-cyano-benzoic acid methyl ester (F9) (300 mg, 50%) as a light tan solid.

Description F10

5-Nitro-N,N-dipropyl-isophthalamic acid methyl ester (F10)

A suspension of 5-nitro-isophthalic acid monomethyl ester (1.0 g, 4.44 mmol, 1 equiv) in CH₂Cl₂ (40 ml) was treated with (COCl)₂ (655 mg, 5.2 mmol, 1.2 equiv) followed by a few drops of DMF. The resulting mixture was stirred for 1 h at room temperature and then dipropylamine (1.65 g, 15 mmol, 3.4 equiv) was added and the resulting solution stirred for a further 30 min. The solution was then washed with 2N aqueous HCl solution (50 ml), saturated aqueous NaHCO₃ solution (50 ml), dried over MgSO₄ and concentrated in vacuo to give 5-nitro-N,N-dipropyl-isophthalamic acid methyl ester (F10) (1.5 g, 110%) as a pale yellow oil.

Description F11

5-Amino-N,N-dipropyl-isophthalamic acid methyl ester (F11)

A mixture of 5-nitro-N,N-dipropyl-isophthalamic acid methyl ester (F10) (1.5 g, 4.9 mmol, 1 equiv), NH₄COOH (3.0 g, 49 mmol, 10 equiv), 10% Pd on charcoal (50% wet, 250 mg, 0.082 equiv w/w), EtOH (20 ml) and H₂O (10 ml) was heated at 50° C. for 90 min. The mixture was cooled to room temperature, filtered through a pad of celite and concentrated in vacuo. The residue was dissolved in AcOEt (200 ml) and the resulting solution was washed with saturated NaHCO₃ solution (100 ml), dried over MgSO₄ and concentrated in vacuo to give 5-amino-N,N-dipropyl-isophthalamic acid methyl ester (F11) (1.2 g, 88%) as a white waxy solid.

Description F12

3-Cyclopent-1-enyl-5-(methanesulfonyl-phenyl-amino)-benzoic tert butyl ester;

3-cyclopent-2-enyl-5-(methanesulfonyl-phenyl-amino)-benzoic acid tert butyl ester;

3-cyclopent-3-enyl-5-(methanesulfonyl-phenyl-amino)-benzoic acid tert butyl ester (F12)

To a solution of 3-bromo-5-(methanesulfonyl-phenyl-amino)-benzoic acid tert-butyl ester (C44) (1 g, 2.35 mmol, 1 equiv) in DMF (10 ml) was added cyclopentene (415 μl, 4.7 mmol, 2 equiv), palladium(II)acetate (25 mg, 0.10 mmol, 0.05 equiv), tri(o-tolyl)phosphine (70 mg, 0.23 mmol, 0.1 equiv) and triethylamine (980 μl, 7 mmol, 3 equiv). The resulting mixture was stirred at 125° C. for 16 h then cooled to room temperature and partitioned between H₂O and Et₂O. The two layers were separated and the organic phase was dried over Na₂SO₄ and concentrated in vacuo. Purification of the residue by flash chromatography (iso-hexane/AcOEt: 4/1) gave a mixture of 3-cyclopent-1-enyl-5-(methanesulfonyl-phenyl-amino)-benzoic tert-butyl ester, 3-cyclopent-2-enyl-5-(methanesulfonyl-phenyl-amino)-benzoic acid tert-butyl ester and 3-cyclopent-3-enyl-5-(methanesulfonyl-phenyl-amino)-benzoic acid tert-butyl ester (F12) (450 mg, 46%) as a colourless oil.

Description F13

3-cyclohex-1-enyl-5-(methanesulfonyl-phenyl-amino)-benzoic tert butyl ester;

3-cyclohex-2-enyl-5-(methanesulfonyl-phenyl-amino)-benzoic acid tert butyl ester;

3-Cyclohex-3-enyl-5-(methanesulfonyl-phenyl-amino)-benzoic acid tert butyl ester (F13)

Description F13 was prepared in an analogous manner to the procedure described for Description F12 using cyclohexene instead of cyclopentene (470 μl, 4.7 mmol, 2 equiv) and using 3-bromo-5-(methanesulfonyl-phenyl-amino)-benzoic acid tert-butyl ester (C44) (1 g, 2.35 mmol, 1 equiv) which yielded 350 mg (40%) of a mixture of 3-cyclohex-1-enyl-5-(methanesulfonyl-phenyl-amino)-benzoic tert butyl ester, 3-cyclohex-2-enyl-5-(methanesulfonyl-phenyl-amino)-benzoic acid tert butyl ester and 3-cyclohex-3-enyl-5-(methanesulfonyl-phenyl-amino)-benzoic acid tert butyl ester (F13) after purification by flash chromatography on silica gel (iso-hexane/EtOAc: 5/1)

Description F14

3-(Methanesulfonyl-phenyl-amino)-5-(2-methyl-propenyl)-benzoic acid tert-butyl ester (F14)

To a solution of 3-bromo-5-(methanesulfonyl-phenyl-amino)-benzoic acid tert-butyl ester (C44) (300 mg, 0.7 mmol, 1 equiv) in DME (7 ml) and H₂O (2 ml) was added tetrakis(triphenylphosphine)-palladium(0) (40 mg, 0.035 mmol, 0.05 equiv), and the suspension was stirred for 30 min. 2,4,6 Triisobutenylcyclotriboroxane-pyridine complex (obtained as described by F. Kerins and D. F. O'Shea in J. Org. Chem, 2002, 67, 4968-4971) (466 mg, 0.7 mmol, 1 equiv) and K₂CO₃ (97 mg, 0.7 mmol, 1 equiv) were added and the resulting mixture was stirred at 90° C. for 4 h, cooled to room temperature and diluted with AcOEt. The organic phase was washed with H₂O, dried over MgSO₄ and concentrated in vacuo. Purification by flash chromatography on silica gel (iso-hexane/AcOEt: 4/1) gave 3-(methanesulfonyl-phenyl-amino)-5-(2-methyl-propenyl) benzoic acid tert-butyl ester (F14) (250 mg, 89%) as a pale yellow oil.

Description F15

3-(Hydroxy-methyl-but-1-ynyl)-5-(methanesulfonyl-phenyl-amino)-benzoic acid tert-butyl ester (F15)

To a solution of 3-bromo-5-(methanesulfonyl-phenyl-amino)-benzoic acid tert-butyl ester (C44) (200 mg, 0.47 mmol, 1 equiv) in DME (2 ml) and H₂O (2 ml) were added K₂CO₃ (136 mg, 1.18 mmol, 2.5 equiv), CuI (9 mg, 0.05 mmol, 0.1 equiv), triphenyl phosphine (14 mg, 0.05 mmol, 0.1 equiv), 10% palladium on charcoal (14 mg, 0.013 mmol, 0.028 equiv) and the solution was stirred at room temperature for 15 min. 2-Methyl-3-butyne-2-ol (113 μl, 1.15 mmol, 2.5 equiv) was added and the resulting mixture was stirred at 90° C. for 16 h then cooled to room temperature. The catalyst was removed by filtration through a pad of celite and the filtrate was diluted with AcOEt. The organic phase was washed with 2N aqueous HCl solution, saturated aqueous NaHCO₃ solution, dried over MgSO₄ and concentrated in vacuo. Purification by flash chromatography on silica gel (iso-hexane/AcOEt: 2/1) gave 3-(hydroxy-methyl-but-1-ynyl)-5-(methanesulfonyl-phenyl-amino)-benzoic acid tert-butyl ester (F15) (150 mg, 75%) as a colourless oil.

Description F16

3-Amino-5-nitro-benzoic acid methyl ester (F16)

To a solution of 3-amino-5-nitro-benzoic acid (65 g, 357 mmol, 1 equiv) in MeOH (650 ml) at 0° C. was added SOCl₂ dropwise (39 ml, 536 mmol, 1.5 equiv). The resulting solution was allowed to warm to room temperature and stirred for 16 h. A further portion of SOCl₂ (10 ml, 137 mmol, 0.4 equiv) was added dropwise and the solution was stirred at room temperature for 5 h, at 50° C. for 2 h and then cooled to room temperature and concentrated in vacuo. The residue was dissolved in AcOEt and the organic phase washed with saturated aqueous NaHCO₃ solution, dried over MgSO₄ and concentrated in vacuo. The solid residue was triturated with AcOEt/iso-hexane to give 3-amino-5-nitro-benzoic acid methyl ester (F16) (55 g, 78%) as a pale yellow solid.

Description F17

3-(4-Chloro-butanoylamino)-5-nitro-benzoic acid methyl ester (F17)

To a solution of 3-amino-5-nitro-benzoic acid methyl ester (F16) (38 g, 194 mmol, 1 equiv) in CH₂Cl₂ (350 ml) was added NEt₃ (32 ml, 230 mmol, 1.2 equiv) followed by 4-chlorobutyryl chloride (24.7 ml, 220 mmol, 1.13 equiv) dropwise over 20 min. The resulting mixture was allowed to warm to room temperature and stirred for 30 min. The organic phase was then washed with 2N aqueous HCl solution, dried over MgSO₄ and concentrated in vacuo. The residue was triturated with iso-hexane and Et₂O to give 3-(4-chloro-butanoylamino)-5-nitro-benzoic acid methyl ester (F17) (56 g, 96%) as a brown solid.

Description F18

3-Nitro-5-(2-oxo-pyrrolidin-1-yl)-benzoic acid methyl ester (F18)

To a solution of 3-(4-chloro-butanoylamino)-5-nitro-benzoic acid methyl ester (F17) (56 g, 186 mmol, 1 equiv) in THF (500 ml) under nitrogen was added portionwise NaH (60% w/w in mineral oil, 8 g, 200 mmol, 1.07 equiv) over 10 min. The resulting mixture was stirred at room temperature for 1 h then cooled to 0° C. and MeOH was added dropwise until bubbling ceased. The solution was concentrated in vacuo and the residue diluted with AcOEt. The organic phase was washed with H₂O, dried over MgSO₄ and concentrated in vacuo. The residue was triturated with iso-hexane to give 3-nitro-5-(2-oxo-pyrrolidin-1-yl)-benzoic acid methyl ester (F18) (38.5 g, 78%) as a light tan solid.

Description F19

3-Amino-5-(2-oxo-pyrrolidin-1-yl)-benzoic acid methyl ester (F19)

A flask was charged with 3-nitro-5-(2-oxo-pyrrolidin-1-yl)benzoic acid methyl ester (F18) (5 g, 19 mmol, 1 equiv), 10% palladium on charcoal (50% wet, 750 mg, 7.5% w/w), NH₄COOH (11.9 g, 190 mmol, 10 equiv) H₂O (30 ml) and MeOH (60 ml). The resulting mixture was stirred at 50° C. for 1.5 h, cooled to room temperature and the catalyst was filtered off through a pad of celite. Most of the MeOH was removed in vacuo and the residue diluted with saturated aqueous NaHCO₃ solution. The aqueous phase was extracted twice with AcOEt. The combined organic phases were dried over MgSO₄ and concentrated in vacuo to give an off white solid. The catalyst was then washed three times with DMF and the combined organic phases concentrated in vacuo. The residue was combined with the material obtained previously and was triturated with Et₂O to give amino-5-(2-oxo-pyrrolidin-1-yl)-benzoic acid methyl ester (F19) (3.9 g, 88%) as a white solid which was used in the next step without further purification.

Description F20

3-(3-Chloro-propane-1-sulfonylamino)-5-nitro-benzoic acid methyl ester (F20)

To a solution of 3-amino-5-nitro-benzoic acid methyl ester (F16) (45 g, 229 mmol, 1 equiv) in CH₂Cl₂ (450 ml) was added pyridine (18.5 ml, 229 mmol, 1 equiv), DMAP (100 mg, 0.8 mmol, catalytic) and 3-chloropropanesulfonyl chloride (28 ml, 230 mmol, 1 equiv). The resulting mixture was stirred for 40 h then diluted with AcOEt. The organic phase was diluted with 2N aqueous HCl solution. The resulting solid was filtered to give 3-(3-chloro-propane-1-sulfonylamino)-5-nitro-benzoic acid methyl ester (23 g, 32%). The filtrate was separated and the organic phase was washed with saturated aqueous NaHCO₃ solution, dried over MgSO₄ and concentrated in vacuo. The residue was triturated with AcOEt and iso-hexane to give a further 50 g (65%) of 3-(3-chloro-propane-1-sulfonylamino)-5-nitro-benzoic acid methyl ester (F20) as a pale brown solid.

[M−H]⁻=334.9, RT=3.11 min

Description F21

3-(1,1-Dioxo-1/⁶-isothiazolidin-2-yl)-5-nitro-benzoic acid methyl ester (F21)

To a solution of 3-(3-chloro-propane-1-sulfonylamino)-5-nitro-benzoic acid methyl ester (F20) (73 g, 217 mmol, 1 equiv) in EtOH (600 ml) was added Et₃N (60 ml, 430 mmol, 2 equiv) and the resulting mixture was refluxed for 3 h, cooled to room temperature and concentrated in vacuo. The residue was dissolved in AcOEt, washed with 2N aqueous HCl solution, dried over MgSO₄ and concentrated in vacuo. The residue was triturated with iso-hexane and AcOEt to give 3-(1,1-dioxo-1/⁶-isothiazolidin-2-yl)-5-nitro-benzoic acid methyl ester (F21) (58 g, 88%) as a pale brown solid. [M+H+NH₃]⁺=318.0, RT=2.78 min

Description F22

3-Amino-5-(1,1-dioxo-1/⁶-isothiazolidin-2-yl)-benzoic acid methyl ester (F22)

A flask was charged with 3-(1,1-dioxo-1/⁶-isothiazolidin-2-yl)-5-nitro-benzoic acid methyl ester (F21) (25 g, 83 mmol, 1 equiv) and 10% palladium (0) on charcoal (50% wet, 5 g, 10% w/w) and EtOH (500 ml). The resulting suspension was stirred under an atmosphere of hydrogen (atmospheric pressure) for 4 h and the catalyst was filtered off through a pad of celite. The catalyst was washed three times with DMF and the combined organic layers were concentrated in vacuo. The residue was dissolved in AcOEt and filtered again through celite in order to remove residual catalyst. The organic phase was concentrated in vacuo. The residue was triturated with Et₂O to give 3-amino-5-(1,1-dioxo-1/⁶-isothiazolidin-2-yl)-benzoic acid methyl ester (F22) (18 g, 80%) as a pale brown solid. [M+H]⁺=271.0, RT=2.16 min

Description F23

3-Bromo-5-nitro-benzoic acid (F23)

To a solution of 3-amino-5-nitro-benzoic acid (17.6 g, 96.6 mmol, 1 equiv) in 48% aqueous HBr solution (180 ml) at 0° C. was added portionwise NaNO₂ (8.67 g, 126 mmol, 1.3 equiv) over 20 min. The temperature was kept below 8° C. during this addition. The resulting mixture was then added to a suspension of CuBr (9.7 g, 67.6 mmol, 0.7 equiv) in 48% aqueous HBr solution (50 ml) at 65° C. over 40 min. The temperature was kept above 60° C. during the addition. The resulting mixture was stirred at 70° C. for 45 min, cooled to room temperature and diluted with 1 L of water. The aqueous phase was extracted three times with Et₂O. The combined organic layers were washed twice with H₂O, dried over MgSO₄ and concentrated in vacuo to give 3-bromo-5-nitro-benzoic acid (F23) (21 g, 88%) as a brown solid. [M−H]⁻=245.7, RT=2.82 min

Description F24

3-Bromo-5-nitro-benzoic acid methyl ester (F24)

3-Bromo-5-nitro-benzoic acid methyl ester (F24) was prepared from 3-bromo-5-nitro-benzoic acid (F23) in accordance with an analogous procedure to that described in F46.

Description F27

3-Amino-5-(methanesulfonyl-phenyl-amino)-benzoic acid methyl ester (F27)

A flask was charged with 3-(methanesulfonyl-phenyl-amino-5-nitro-benzoic acid methyl ester (F26) (400 mg, 1.14 mmol, 1 equiv), 10% palladium on charcoal (50% wet, 40 mg, 5% w/w), NH₄COOH (718 mg, 11.4 mmol, 10 equiv) H₂O (2 ml) and EtOH (10 ml). The resulting mixture was stirred at 50° C. for 2 h, cooled to room temperature and the catalyst was filtered off through a pad of celite. Most of the EtOH was removed in vacuo and the residue diluted with H₂O and AcOEt. The layers were separated. The organic phase was dried over MgO₄ and concentrated in vacuo to give 3-amino-5-(methanesulfonyl-phenyl-amino)-benzoic acid methyl ester (F27) (330 mg, 90%) as a pale orange solid.

Description F28

5-Ethoxy-isophthalic acid dimethyl ester (F28)

K₂CO₃ (31.6 g, 223 mmol, 2.23 equiv) and iodoethane (17.8 ml, 230 mmol, 2.3 equiv) were added to a solution of 5-hydroxy-isophthalic acid dimethyl ester (21 g, 100 mmol, 1 equiv) in acetone (500 ml) at room temperature. The resulting solution was refluxed for 16 h, then cooled to room temperature and concentrated in vacuo. The residue was partitioned between H₂O and AcOEt. The aqueous phase was extracted with AcOEt and the combined organic layers were washed with 2N aqueous NaOH solution and brine, dried over MgSO₄ and concentrated in vacuo to give 5-ethoxy-isophthalic acid dimethyl ester (F28) (23 g, 96%) as a white solid. RT=3.13 min

Description F29

5-Ethoxy-isophthalic acid monomethyl ester (F29)

To a solution of 5-ethoxy-isophthalic acid dimethyl ester (F28) (22 g, 92.4 mmol, 1 equiv) in MeOH (440 ml) was added 1N aqueous NaOH solution (87.8 ml, 87.8 mmol, 0.95 equiv) and the resulting solution was stirred at room temperature for 17 h. Most of the MeOH was removed in vacuo and the residue was partitioned between AcOEt and 1N aqueous NaOH solution. The aqueous layer was extracted with AcOEt, acidified to pH 1 and re-extracted with AcOEt. The second organic extract was dried over MgSO₄ and concentrated in vacuo to give 5-ethoxy-isophthalic acid monomethyl ester (F29) (17 g, 82%) as a white solid. [M+H+NH₃]⁺=242.0, RT=2.79 min

Description F30

3-Benzyloxycarbonylamino-5-ethoxy-benzoic acid methyl ester (F30)

NEt₃ (14.2 ml, 102 mmol, 1.3 equiv) and diphenylphosphoryl azide (22 ml, 102 mmol, 1.3 equiv) were added to a suspension of 5-ethoxy-isophthalic acid monomethyl ester (F29) (17.6 g, 78.6 mmol, 1 equiv) in toluene (250 ml) and the mixture heated at 80° C. for 3 h. Benzyl alcohol (12 ml, 118 mmol, 1.5 equiv) was added and the resulting mixture was refluxed for 4 h, cooled to room temperature and concentrated in vacuo. The residue was dissolved in AcOEt (300 ml) and the resulting solution was washed with 2N aqueous HCl solution (100 ml) followed by saturated aqueous NaHCO₃ solution (100 ml), dried over MgSO₄ and concentrated in vacuo. The residue was triturated with Et₂O to give 3-benzyloxycarbonylamino-5-ethoxy-benzoic acid methyl ester (F30) (15 g, 62%) as a white solid. [M−H]⁻=328.1, RT=3.46 min

Description F31

3-Amino-5-ethoxy-benzoic acid methyl ester (F31)

A mixture of 3-benzyloxycarbonylamino-5-ethoxy-benzoic acid methyl ester (F30) (15 g, 45.5 mmol, 1 equiv), 10% palladium on charcoal (50% wet, 1.5 g, 5% w/w) and NH₄COOH (15 g, 455 mmol, 10 equiv) H₂O (50 ml) and MeOH (200 ml) was stirred at 50° C. for 2 h. The mixture was cooled to room temperature and the catalyst was filtered off through a pad of celite. Most of the MeOH was removed in vacuo and the residue was partitioned between saturated aqueous NaHCO₃ solution and AcOEt. The aqueous phase was re-extracted with AcOEt. The combined organic phases were dried over MgSO₄ and concentrated in vacuo to give 3-amino-5-ethoxy-benzoic acid methyl ester (F31) (8.8 g, 99%) as a pale green solid. [M+H]⁺=196.1, RT=2.49 min

Description F32

5-Dimethylthiocarbamoyloxy-isophthalic acid dimethyl ester (F32)

To a solution of 5-hydroxy-isophthalic acid dimethyl ester (21 g, 100 mmol, 1 equiv) in DMF (300 ml) at room temperature was added DABCO (14.6 g, 130 mmol, 1.3 equiv) followed by dimethylthiocarbamoyl chloride (14.8 g, 120 mmol, 1.2 equiv). The resulting mixture was stirred at room temperature for 16 h and at 60° C. for 2 h, then cooled to room temperature and concentrated in vacuo. The residue was partitioned between AcOEt and H₂O and the aqueous phase re-extracted with AcOEt. The combined organic solution was washed sequentially with 5% aqueous citric acid solution, 2N aqueous NaOH solution and brine, then dried over MgSO₄ and concentrated in vacuo to give 5-dimethylthiocarbamoyloxy-isophthalic acid dimethyl ester (F32) (23.5 g, 79%) as a pale yellow oil. [M+H]⁺=298.0, RT=3.06 min

Description F33

5-Dimethylcarbamoylsulfanyl-isophthalic acid dimethyl ester (F33)

5-Dimethylthiocarbamoyloxy-isophthalic acid dimethyl ester (F32) (15.5 g, 52.2 mmol, 1 equiv) was stirred at 200° C. for 24 h under nitrogen then cooled to room temperature. Purification by flash chromatography on silica gel (iso-hexane/AcOEt: 4/1 then 3/1) gave 5-dimethylcarbamoylsulfanyl-isophthalic acid dimethyl ester (F33) (7.0 g, 45%) and recovered 5-dimethylthiocarbamoyloxy-isophthalic acid dimethyl ester (F32) (2.77 g, 18%), both as white solids. [M+H]⁺=298.0, RT=2.92 min

Description F34

5-Dimethylcarbamoylsulfanyl-isophthalic acid monomethyl ester (F34)

To a solution of 5-dimethylcarbamoylsulfanyl-isophthalic acid dimethyl ester (F33) (6 g, 20.2 mmol, 1 equiv) in THF (100 ml) at room temperature was added 2N aqueous NaOH solution (9.6 ml, 19.2 mmol, 0.95 equiv). The resulting mixture was stirred for 11 h and then partitioned between AcOEt and H₂O. The two layers were separated and the aqueous phase extracted with AcOEt. After acidification to pH 1, the aqueous phase was extracted twice with AcOEt. The organic solution was dried over MgSO₄ then concentrated in vacuo to give 5-dimethylcarbamoylsulfanyl-isophthalic acid monomethyl ester (F34) (4.54 g, 79%) as a white solid.

Description F35

tert-Butoxycarbonylamino-dimethylcarbamoylsulfanyl-benzoic acid methyl ester (F35)

To a solution of 5-dimethylcarbamoylsulfanyl-isophthalic acid monomethyl ester (F34) (4.56 g, 16.1 mmol, 1 equiv) in toluene (100 ml) was added triethylamine (6.7 ml, 48 mmol, 3 equiv) and diphenylphosphoryl azide (5.2 ml, 24 mmol, 1.5 equiv). The resulting mixture was stirred under nitrogen at 80° C. for 3 h and then tert-butanol (4.6 ml, 48 mmol, 3 equiv) was added. The solution was stirred at 80° C. for another 16 h then cooled to room temperature and concentrated in vacuo. The crude product was dissolved in AcOEt and the resulting solution washed sequentially with 2N aqueous NaOH solution, 2N aqueous HCl solution and brine, dried over MgSO₄ and concentrated in vacuo. Purification by flash chromatography on silica gel (iso-hexane/AcOEt: 3/1 to 6/4) gave tert-butoxycarbonylamino-dimethylcarbamoylsulfanyl-benzoic acid methyl ester (F35) (2.24 g, 40%). as a white solid.

Description F36

3-tert-Butoxycarbonylamino-5-mercapto-benzoic acid (F36)

To a solution of tert-butoxycarbonylamino-dimethylcarbamoylsulfanyl-benzoic acid methyl ester (F35) (2.24 g, 6.3 mmol, 1 equiv) in MeOH (30 ml) and H₂O (23 ml) was added 2N aqueous NaOH solution (7 ml, 14 mmol, 2.2 equiv). The resulting mixture was refluxed for 3 h and then cooled to room temperature and concentrated in vacuo. The residue was partitioned between AcOEt and 1N aqueous NaOH solution. The aqueous phase was acidified to pH 1 and extracted twice with AcOEt. The combined organic solutions were dried over MgSO₄ then concentrated in vacuo to give 3-tert-butoxycarbonylamino-5-mercapto-benzoic acid (F36) (1.54 g, 90%) as a white solid.

Description F37

3-tert-Butoxycarbonylamino-5-methylsulfanyl-benzoic acid methyl ester (F37)

To a solution of 3-tert-butoxycarbonylamino-5-mercapto-benzoic acid (F36) (0.68 g, 2.52 mmol, 1 equiv) in acetone (15 ml) was added K₂CO₃ (3.5 g, 25.3 mmol, 10 equiv) and iodomethane (473 μl, 7.59 mmol, 3 equiv). The resulting mixture was stirred at 50° C. for 2 h, cooled to room temperature and concentrated in vacuo. The residue was partitioned between AcOEt and H₂O. The organic layer was washed with H₂O and brine, dried over MgSO₄ and concentrated in vacuo. Purification by flash chromatography on silica gel (iso-hexane/AcOEt: 85/15) gave 3-tert-butoxycarbonylamino-5-methylsulfanyl-benzoic acid methyl ester (F37) (0.47 g, 63%) as a white solid. [M−H]⁻=296.1, RT=3.51 min

Description F38

3-tert-Butoxycarbonylamino-5-ethylsulfanyl-benzoic acid ethyl ester (F38)

Description F38 was prepared in an analogous manner to that described for Description F37, using iodoethane as the alkylating agent, from 0.68 g (2.53 mmol) of 3-tert-butoxycarbonylamino-5-methylsulfanyl-benzoic acid methyl ester (F36), which yielded 3-tert-butoxycarbonylamino-5-ethylsulfanyl-benzoic acid ethyl ester (F38) (0.58 g, 71%) as a white solid. [M−H]⁻=324.2, RT=3.79 min

Description F39

3-Amino-5-methylsulfanyl-benzoic acid methyl ester hydrochloride (F39)

3-tert-Butoxycarbonylamino-5-methylsulfanyl-benzoic acid methyl ester (F37) (0.54 g, 1.82 mmol, 1 equiv) was dissolved in dioxan (2 ml) and 4M HCl in dioxan (16 mmol, 4 ml, 8.8 equiv) was added. The solution was stirred at room temperature for 2 h allowing the hydrochloride salt of the amine to precipitate. This precipitate was filtered off, washed with Et₂O and dried giving 3-amino-5-methylsulfanyl-benzoic acid methyl ester hydrochloride (F39) (0.224 g, 52%).

[M+H]⁺=198.1

RT=2.68 min

Description F40

3-Amino-5-ethylsulfanyl-benzoic acid ethyl ester hydrochloride (F40)

F40 was prepared in an analogous manner to Intermediate 39 from 0.57 g (1.75 mmol) 3-tert-butoxycarbonylamino-5-ethylsulfanyl-benzoic acid ethyl (F38) which yielded F40 as a white solid; 0.335 g (73%). [M+H]⁺=226.1, RT=3.13 min

Description F41

3-Bromo-5-iodo-benzoic acid tert-butyl ester (F41)

To a solution of 3-bromo-5-iodo-benzoic acid (50 g, 153 mmol, 1 equiv) in CH₂Cl₂ (500 ml) was added 1-(3-dimethylaminopropyl)₃-ethyl-carbodiimide hydrochloride (30.8 g, 160 mmol, 1.05 equiv), DMAP (14 g, 114 mmol, 0.75 equiv) and tert-butanol (90 ml, 917 mmol, 6 equiv). The resulting mixture was stirred at room temperature for 48 h. DMAP (4.67 g, 38 mmol, 0.25 equiv) was then added and the solution was stirred for another 24 h then concentrated in vacuo. The residue was dissolved in AcOEt and washed sequentially with 2N aqueous HCl solution, 1N aqueous NaOH solution and brine, dried over MgSO₄ and concentrated in vacuo to give 3-bromo-5-iodo-benzoic acid tert-butyl ester (F41) (50.6 g, 86%) as a brown solid.

The following compounds were made in an analogous manner to that described for Description F41:

| | Starting |
|---|---|
| Description | Material |
| 3-Chloro-2-fluoro-benzoic acid tert-butyl ester (F42) | 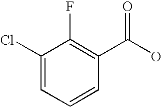 |
| 3-Bromo-4-fluoro-benzoic acid tert-butyl ester (F43) | 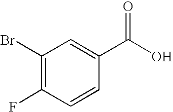 |
| 3-Nitro benzoic acid tert-butyl ester (F44) | 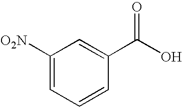 |

Description F45
3-Amino-benzoic acid tert-butyl ester (F45)

To a solution of 3-nitro-benzoic acid tert-butyl ester (F44) (1 g, 4.5 mmol, 1 equiv) in EtOH (30 ml) and H₂O (6 ml) was added 10% palladium on charcoal (50% wet, 100 mg, 5% w/w) and NH₄COOH (2.8 g, 45 mmol, 10 equiv). The resulting mixture was stirred at 50° C. for 2 h then cooled to room temperature. The catalyst was removed by filtration over a pad of celite. Most of the EtOH was removed in vacuo and the residue dissolved in AcOEt. The organic phase was washed with saturated NaHCO₃ solution, dried over MgSO₄ and concentrated in vacuo to give 3-amino-benzoic acid tert-butyl ester (F45) (796 mg, 92%) as a colorless oil,

Description F46

3-Amino-4-chloro-benzoic acid methyl ester (F46)

To a solution of 3-amino-4-chloro-benzoic acid (1.7 g, 10 mmol, 1 equiv) in MeOH (70 ml) at 0° C. was added SOCl₂ (1.46 ml, 20 mmol, 2 equiv). The resulting solution was refluxed for 16 h, cooled to room temperature and concentrated in vacuo. The residue was diluted with AcOEt and washed twice with 2N aqueous NaOH solution then brine, dried over MgSO₄ and concentrated in vacuo to give 3-amino-4-chloro-benzoic acid methyl ester (F46) (1.8 g, 97%) as a pale yellow oil.

Description F47

4-((Z/E)-But-2-enylamino)-3,5-diiodo-benzoic acid ethyl ester (F47)

To a solution of 4-amino-3,5-diiodo-benzoic acid ethyl ester (commercially available from Maybridge) (72.6 g, 0.17 mmol, 1 equiv) in DMF (450 ml) at 0° C. under nitrogen was added NaH (60% in mineral oil, 7.3 g, 0.18 mmol, 1.05 equiv) portionwise over 2 min. After 10 min crotyl bromide (21.5 ml, 0.21 mmol, 1.2 equiv) in DMF (50 ml) was added via cannula over 5 min and the resulting mixture was allowed to warm to room temperature over 30 min. 5 ml of EtOH were added and the mixture was concentrated in vacuo. The residue was dissolved in AcOEt and the organic phase was washed with H₂O. The aqueous phase was extracted with AcOEt and the combined organic phases were washed with brine, dried over MgSO₄ and concentrated in vacuo to give the title compound (F47) (82 g, 100%) as a pink solid which was used in the next step without further purification. [M+H]⁺=472.0, RT=4.93 min.

Description F48

3-Ethyl-7-iodo-1H-indole-5-carboxylic acid ethyl ester (F48)

To a solution of 4-((Z/E)but-2-enylamino)-3,5-diiodo-benzoic acid ethyl ester (F47) (15 g, 31.8 mmol, 1 equiv) in DMF (150 ml) at room temperature under nitrogen were added Pd(OAc)₂ (357 mg, 1.6 mmol, 0.05 equiv), NaCOOH (6.5 g, 95.6 mmol, 3 equiv), Na₂CO₃ (8.4 g, 79.6 mmol, 2.5 equiv) and Nbu₄Cl (8.0 g, 35.0 mmol, 1.1 equiv). The resulting suspension was stirred under nitrogen at 80° C. for 30 min then cooled to room temperature and concentrated in vacuo. The residue was partitioned between AcOEt and H₂O and the two phases were separated. The organic phase was dried over MgSO₄ and concentrated in vacuo. Purification of the residue by flash chromatography on silica gel (isohexane/AcOEt: 9/1) gave the title compound (F48) (6.3 g, 58%) as a white solid. [M+H]⁺=344.0, RT=3.86 min.

Description F49

7-Benzyloxycarbonylamino-3-ethyl-1H-indole-5-carboxylic acid ethyl ester (F49)

To a solution of 3-ethyl-7-iodo-1H-indole-5-carboxylic acid ethyl ester (F48) (850 mg, 2.48 mmol, 1 equiv) in toluene (20 ml) at room temperature under nitrogen were added benzyl carbamate (562 mg, 3.72 mmol, 1.5 equiv), copper iodide (24 mg, 0.13 mmol, 0.05 equiv) K₃PO₄ (1.05 g, 4.8 mmol, 2 equiv) and N,N′-dimethylethylenediamine (26 μl, 0.25 mmol, 0.1 equiv) and the resulting suspension was stirred at 100° C. for 30 min then cooled to room temperature and concentrated in vacuo. The residue was partitioned between AcOEt and H₂O and the two phases were separated. The organic phase was dried over MgSO₄ and concentrated in vacuo. Purification of the residue by flash chromatography on silica gel (isohexane/AcOEt: 9/1) gave the title compound (F49) (250 mg, 27%) as an off white solid. [M+H]⁺=367.1, RT=3.73 min.

Description F50

7-Amino-3-ethyl-1H-indole-5-carboxylic acid ethyl ester (F50)

To a solution of 7-benzyloxycarbonylamino-3-ethyl-1H-indole-5-carboxylic acid ethyl ester (F49) (250 mg, 0.68 mg, 1 equiv) in EtOH (10 ml) were added NH₄COOH (431 mg, 6.8 mmol, 10 equiv), H₂O (2 ml), Pd (10% w/w on charcoal, 50 mg, 0.02 equiv w/w) and the resulting mixture was stirred at 70° C. for 1.5 h. Another 200 mg of Pd (10% w/w on charcoal, 0.08 equiv w/w) were then added and the resulting mixture stirred at 70° C. for another 30 min then cooled to room temperature. The catalyst was filtered off through a pad of celite and most of the EtOH was removed in vacuo. The residue was partitioned between AcOEt and H₂O and the two phases were separated. The organic phase was dried over MgSO₄ and concentrated in vacuo to give the title compound (F50) (150 mg, 95%) as an off white solid which was used in the next step without further purification. [M+H]⁺=233.1, RT=3.19 min.

Description F51

1,1,5-Trimethyl-hexylamine (F51)

Description F51 was obtained according to S. S. Berg and D. T. Cowling, J. Chem. Soc. (C) 1971, 1653-1658.

Description 1

3-Methanesulfonylamino-benzoic acid methyl ester (D1)

To a solution of 3-amino-benzoic acid methyl ester (10 g, 66 mmol, 1 equiv) in CH₂Cl₂ (100 ml) at room temperature were added pyridine (5.86 ml, 72.6 mmol, 1.1 equiv), CH₃SO₂Cl (5.37 ml, 70 mmol, 1.06 equiv) and DMAP (1 g, 8.2 mmol, 0.12 equiv) and the resulting mixture was stirred at this temperature for 16 h then concentrated in vacuo. The residue was diluted with AcOEt and the organic phase was washed with 2N HCl aqueous solution, saturated NaHCO₃ solution and brine, dried over MgSO₄ and concentrated in vacuo to give 3-methanesulfonylamino-benzoic acid methyl ester (D1) (13.4 g, 89%) as a pale yellow oil. [M−H]⁻=228.0, RT=2.40 min

The following compounds were prepared in accordance with the procedure described in Description 1 from the appropriate aniline starting material:

| Description | Starting Material |
|---|---|
| 3-(1,1-Dioxo-1I^{β}-isothiazolidin-2-yl)-5- | F22 |
| methanesulfonylamino-benzoic | |
| acid methyl ester (D2) | |
| 5-Methanesulfonylamlno-N,N-dipropyl- | F11 |
| isophthalamic acid methyl ester (D3) | |
| 3-Ethoxy-5-methanesulfonylamino- | F31 |
| benzoic acid methyl ester | |
| 3-Methanesulfonylamino-benzoic acid ethyl ester (D5) | 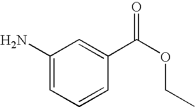 |
| 3-Methanesulfonylamino-benzoic acid | F45 |
| tert-butyl ester (06) | |
| 3-Cyano-5-methanesulfonylamino- | F9 |
| benzoic acid methyl ester (D7) | |
| 3-Methanesulfonylamino-2- methyl-benzoic acid methyl ester (D5) | 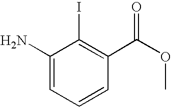 |
| 3-Methanesulfonylamino-4- methyl-benzoic acid methyl ester (D10) | 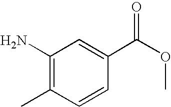 |
| 4-Chloro-3-methanesulfonylamino- | F46 |
| benzoic acid methyl ester (D12) | |
| 5-Methanesulfonylamino-2- methyl-benzoic acid methyl ester (D13) | 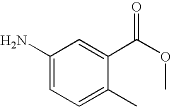 |
| 3-Methanesulfonylamino-5- | F39 |
| methylsulfanyl-benzoic acid methyl | |
| ester (D14) | |
| 3-Methanesulfonylamino-5- | F40 |
| ethylsulfanyl-benzoic acid methyl | |
| ester (D15) | |
| 3-Methanesulfonylamino-5- | F19 |
| (2-oxo-pyrrolidin-1-yl)-benzoic acid | |
| methyl ester (D18) | |
| 3-Ethyl-7-methanesulfonylamino- | F50 |
| 1H-indole-5-carboxylic acid | |
| ethyl ester (D50) | |

Description 16
5-Methanesulfonylamino-nicotinic acid ethyl ester (D16)

D16 was obtained in an analogous manner to the procedure described in Description 17 from 5-amino-nicotinic acid ethyl ester (which was prepared in accordance with Jensen, H. H. et al Chem. Europ. J 2002, 8 (5), 1218-1226).

Description 17

2-Methanesulfonylamino-isonicotinic acid ethyl ester (D17)

To a solution of 2-amino-isonicotinic acid ethyl ester (obtained according to Seewood, D. L. and all, J. Med. Chem. 2001,44(1), 78-93) (1 g, 6.02 mmol, 1 equiv) in CH₂Cl₂ (60 ml) at room temperature were added pyridine (700 μl, 8.6 mmol, 1.4 equiv), CH₃SO₂Cl (606 μl, 0.78 mmol, 1.25 equiv) and DMAP (100 mg, 0.82 mmol, 0.13 equiv) and the resulting mixture was stirred at this temperature for 16 h then partitioned between CH₂Cl₂ and NaHCO₃ saturated aqueous solution. The two layers were separated and the organic phase was washed water, dried over MgSO₄ and concentrated in vacuo. The residue was triturated with Et₂O and the precipitate filtered off to give 2-methanesulfonylamino-isonicotinic acid ethyl ester (D17) (1.6 g, 110%) as an orange solid.

Description 27

3-(4-Trifluoromethyl-phenylamino)-benzoic acid ethyl ester (D27)

A flask was charged under nitrogen with 4-bromo-trifluoromethyl-benzene (1.25 ml, 8.9 mmol, 1 equiv), Cs₂CO₃ (4.4 g, 13.4 mmol, 1.5 equiv), palladium(II)acetate (101 mg, 0.45 mmol, 0.05 equiv), rac-BINAP (416 mg, 0.67 mmol, 0.075 equiv) and toluene (30 ml). 3-Amino-benzoic acid ethyl ester (1.6 ml, 10.7 mmol, 1.2 equiv) was then added via syringe and the resulting mixture was stirred at 100° C. for 3 h then cooled to room temperature and diluted with AcOEt. The organic phase was washed with H₂O, 2N HCl aqueous solution then brine, dried over MgSO₄ and concentrated in vacuo. Purification of the residue by flash chromatography on silica gel (iso-hexane/AcOEt: 4/1) gave 3-(4-trifluoromethyl-phenylamino)benzoic acid ethyl ester (D27) (600 mg, 21%) as an off-white solid.

The following compounds were prepared in an analogous manner to that described for Description 27 from the appropriate aniline and aryl halide:

| | | |
|---|---|---|
| Description | Aniline | Aryl halide |

| | | |
|---|---|---|
| 2-Fluoro-3-phenylamino-benzoic acid tert-butyl ester (D31) | 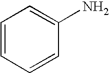 | F42 |
| 4-Fluoro-3-phenylamino-benzoic acid tert-butyl ester (D32) | 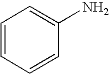 | F43 |
| 3-Phenylamino-benzoic acid methyl ester (D49) | 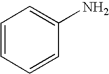 | 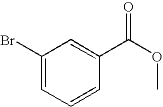 |
| 3-(2-Methoxy-phenylamino)-benzoic acid methyl ester (D33) | 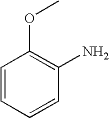 | 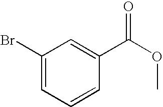 |
| 3-(3-Methoxy-phenylamino)-benzoic acid methyl ester (D34) | 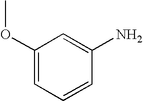 | 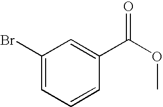 |
| 3-(4-Methoxy-phenylamino)-benzoic acid methyl ester (D35) | 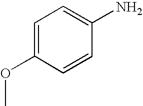 | 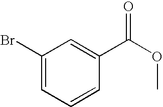 |
| 3-(2-Chloro-phenylamino)-benzoic acid methyl ester (D36) | 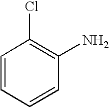 | 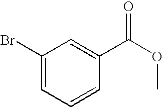 |
| 3-(3-Chloro-phenylamino)-benzoic acid methyl ester (D37) | 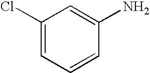 | 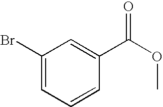 |
| 3-(4-Chloro-phenylamino)-benzoic acid methyl ester (D38) | 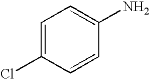 | 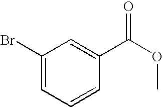 |
| 3-(3,4-Dichloro-phenylamino)-benzoic acid methyl ester (D39) | 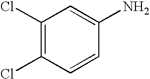 | 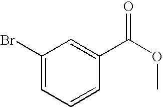 |
| 3-(3,5-Dichioro-phenylamino)-benzoic acid methyl ester (D40) | 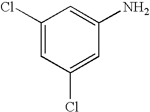 | 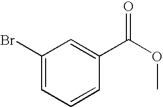 |
| 3-(2-Cyano-phenylamino)-benzoic acid methyl ester (D41) | 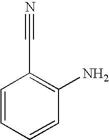 | 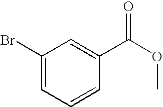 |
| 3-(3-Cyano-phenylamino)-benzoic acid methyl ester (D42) | 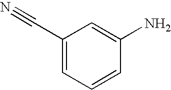 | 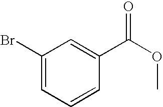 |
| 3-(4-Cyano-phenylamino)-benzoic acid methyl ester (D43) | 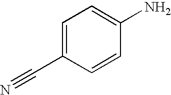 | 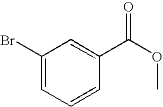 |
| 3-Bromo-5-phenylamino-benzoic acid tert-butyl ester (D44) | 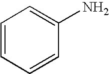 | F41 |
| 3-(Naphthalen-1-ylamino)-benzoic acid tert-butyl ester (D45) | 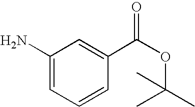 | F45 |
| 3-Nitro-5-phenylamino-benzoic acid methyl ester (F25) | 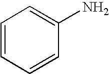 | F24 |

Description 47

3-(Pyridin-2-ylamino)-benzoic acid tert-butyl ester (D47)

A flask was charged under nitrogen with 3-bromo-pyridine (908 μl, 9.42 mmol, 1 equiv), t-BuONa (1.36 g, 14.1 mmol, 1.5 equiv), tris(dibenzylideneacetone)dipalladium(0) (86 mg, 0.09 mmol, 0.01 equiv), 1,3-bis-(2,6-diisopropyl-phenyl)-4,5-dihydro-3H-imidazol-1-ium chloride (80 mg, 0.19 mmol, 0.02 equiv) and dioxan (30 ml). 3-Amino-benzoic acid tert-butyl ester (2 g, 10.4 mmol, 1.1 equiv) was then added and the resulting mixture was stirred at 100° C. for 16 h then cooled to room temperature and diluted with AcOEt. The organic phase was washed with 2N HCl aqueous solution. The aqueous phase was basified to pH 13 with 2N NaOH aqueous solution and extracted twice with AcOEt. The combined organic phases were dried over MgSO₄ and concentrated in vacuo. Purification of the residue by flash chromatography on silica gel (iso-hexane/AcOEt: 1/2) gave 3-(pyridin-2-ylamino)-benzoic acid tert-butyl ester (D47) (420 mg, 16%) as a pale yellow oil.

The following compounds were obtained in an analogous manner to that described for Description 47 from the appropriate aniline and aryl halide:

| | | |
|---|---|---|
| Description | Aryl halide | Aniline |

| | | |
|---|---|---|
| 3-(Pyridin-2-ylamino)-benzoic acid tert-butyl ester (D46) | 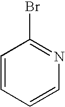 | F45 |
| 3-(Pyridin-4-ylamino)-benzoic acid tert-butyl ester (D48) | 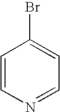 | F45 |

Preparation of Esters
Ester 1
3-(Methanesulfonyl-phenyl-amino)-benzoic acid methyl ester (C1)

To a solution of 3-methanesulfonylamino-benzoic acid methyl ester (D1) (2 g, 8.7 mmol, 1 equiv) in CH₂Cl₂ (25 ml) were added phenylboronic acid (2.1 g, 17.5 mmol, 2 equiv), Cu(OAc)₂ (1.9 g, 10 mmol, 1.15 equiv), NEt₃ (2.4 ml, 17.5 mmol, 2 equiv) and powered activated 4A molecular sieves (1 g, 50% w/w). The resulting mixture was stirred at room temperature for 16 h then the molecular sieves were filtered off through a pad of celite and the organic phase was washed with 2N HCl aqueous solution, NaHCO₃ saturated aqueous solution, dried over MgSO₄ and concentrated in vacuo. Purification of the residue by flash chromatography on silica gel (iso-hexane/AcOEt: 6/1) gave 3-(methanesulfonyl-phenyl-amino)-benzoic acid methyl ester (C1) (680 mg, 26%) as a white solid. [M+H—SO₂Me]⁺=227.0, R.T.=3.04

The following esters were prepared in an analogous manner to the procedure described for Ester 1 from the corresponding N-aryl sulphonamide and boronic acid starting materials:

| | N-aryl | |
|---|---|---|
| Ester | sulfonamide | Boronic acid |
| 3-(Dioxo-1I^{β}-isothiazolidin-2- yl)-5-(methanesulfonyl- phenyl-amino)-benzoic acid methyl ester (C2) | D2 | 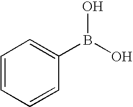 |
| 3-(Methanesulfonyl-phenyl- amino)-5-N,N-dipropyl-iso- phthalamic acid methyl ester (C3) | D3 | 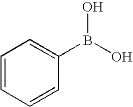 |
| 3-Ethoxy-5-(methane- sulfonyl-phenyl-amino)- benzoic acid methyl ester (C4) | D4 | 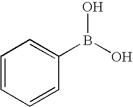 |
| 3-(Methanesulfonyl-naphtha- len-2-yl-amino)-benzoic acid methyl ester (C5) | D5 | 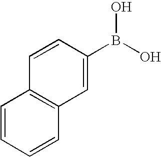 |
| 3-[(4-Fluoro-phenyl)- methanesulfonyl-amino]- benzoic acid tert-butyl ester (C6) | D6 | 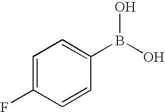 |
| Cyano-(methanesulfonyl- phenyl-amino)-benzoic acid methyl ester (C7) | D7 | 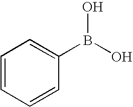 |
| 3-[(3-Fluoro-phenyl)- methanesulfonyl-amino]- benzoic acid tert-butyl ester (C8) | D8 | 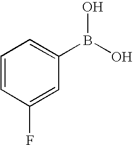 |
| 3-[(3,5-Difluoro-phenyl)- methanesulfonyl-amino]- benzoic acid tert-butyl ester (C9) | D9 | 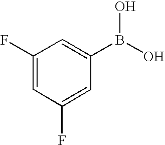 |
| 3-(Methanesulfonyl-phenyl- amino)-2-methyl-benzoic acid methyl ester (C10) | D10 | 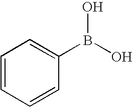 |
| 3-(Methanesulfonyl-phenyl- amino)-4-methyl-benzoic acid methyl ester (C11) | D11 | 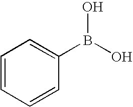 |
| 4-Chloro-3-(methanesulfonyl- phenyl-amino)-benzoic acid methyl ester (C12) | D12 | 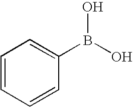 |
| 3-(Methanesulfonyl-phenyl- amino)-5-methyl-benzoic acid methyl ester (C13) | D13 | 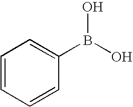 |
| 3-(Methanesulfonyl-phenyl- amino)-5-methylsulfanyl- benzoic acid methyl ester (C14) | D14 | 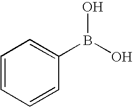 |
| 3-(Methanesulfonyl-phenyl- amino)-5-ethylsulfanyl- benzoic acid methyl ester (C15) | D15 | 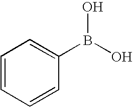 |

Ester 16
5-(Methanesulfonyl-phenyl-amino)-nicotinic acid ethyl ester (C16)

Ester 16 was prepared in an analogous manner to Ester 17 from 5-methanesulfonylamino-nicotinic acid ethyl ester (D16).

Ester 17

2-(Methanesulfonyl-phenyl-amino)-isonicotinic acid ethyl ester (C17)

To a solution of 2-methanesulfonylamino-isonicotinic acid ethyl ester (D17) (1.47 g, 6.02 mmol, 1 equiv) in CH₂Cl₂ (25 ml) were added phenylboronic acid (2.2 g, 18.0 mmol, 3 equiv), Cu(OAc)₂ (2.19 g, 12.06 mmol, 2 equiv), NEt₃ (2.5 ml, 18.0 mmol, 3 equiv) and powered activated 4A molecular sieves (1 g, 70% w/w). The resulting mixture was stirred at room temperature for 16 h then the molecular sieves were filtered off through a pad of celite and the resulting solution was concentrated in vacuo. The residue was partitioned between AcOEt and saturated NaHCO₃ aqueous solution and the two layers were separated. the organic phase was washed with brine, dried over MgSO₄ and concentrated in vacuo. Purification of the residue by flash chromatography on silica gel (iso-hexane/AcOEt: 2/1) gave 2-(methanesulfonyl-phenyl-amino)-isonicotinic acid ethyl ester (C17) (394 mg, 21%) as a yellow solid.

The following esters were prepared in an analogous manner to the procedure described for Ester 1 from the corresponding N-aryl sulphonamide and boronic acid starting materials:

| | N-aryl | |
|---|---|---|
| | sulfona- | Boronic |
| Ester | mide | acid |
| 3-[(4-Fluoro-phenyl)- methanesulfonyl-amino]- 5-(2-oxo-pyrrolidin-1-yl)- benzoic acid methyl ester (C18) | D18 | 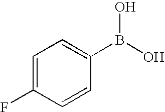 |
| 3-[(3-Fluoro-phenyl)- methanesulfonyl-amino]- 5-(2-oxo-pyrrolidin-1-yl)- benzoic acid methyl ester (C19) | D18 | 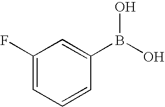 |
| 3-[(3-Methoxy-phenyl)- methanesulfonyl-amino]- 5-(2-oxo-pyrrolidin-1-yl)- benzoic acid methyl ester (C20) | D18 | 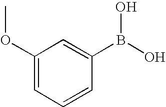 |
| 3-[(4-Methoxy-phenyl)- methanesulfonyl-amino]- 5-(2-oxo-pyrrolidin-1-yl)- benzoic acid methyl ester (C21) | D18 | 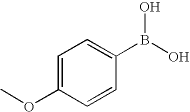 |
| 3-[(3,4-Difluoro-phenyl)- methanesulfonyl-amino]- 5-(2-oxo-pyrrolidin-1-yl)- benzoic acid methyl ester (C22) | D18 | 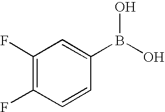 |
| 3-[(3,5-Difluoro-phenyl)- methanesulfonyl-amino]- 5-(2-oxo-pyrrolidin-1-yl)- benzoic acid methyl ester (C23) | D18 | 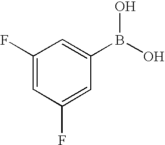 |
| 3-[(3-Cyano-phenyl)- methanesulfonyl-amino]- 5-(2-oxo-pyrrolidin-1-yl)- benzoic acid methyl ester (C24) | D18 | 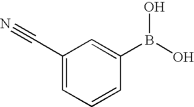 |
| 3-[(4-Cyano-phenyl)- methanesulfonyl-amino]- 5-(2-oxo-pyrrolidin-1-yl)- benzoic acid methyl ester (C25) | D18 | 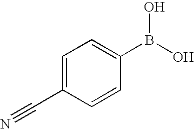 |
| 3-[(3-Carbamoyl-phenyl)- methanesulfonyl-amino]- 5-(2-oxo-pyrrolidin-1-yl)- benzoic acid methyl ester (C26) | D18 | 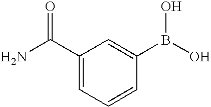 |
| Ethyl-(methanesulfonyl- phenyl-amino)1H-indole-5- carboxylic acid ethyl ester (C59) | D50 | 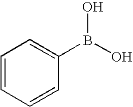 |

Ester 27
3-[Methanesulfonyl-(4-trifluoromethyl-phenyl)-amino]-benzoic acid ethyl ester (C27)

To a solution of 3-(4-trifluoromethyl-phenylamino)-benzoic acid ethyl ester (D27) (600 mg, 1.94 mmol, 1 equiv) in THF (10 ml) at −78° C. was added LDA (2M in THF/n-heptane, 2.45 ml, 4.9 mmol, 2.5 equiv) dropwise and the resulting mixture was stirred at this temperature for a further 5 min. Methanesulfonyl chloride (376 μl, 4.9 mmol, 2.5 equiv) was then added dropwise and the resulting mixture was allowed to warm to room temperature over 30 min then diluted with AcOEt. The organic phase was washed with 2N HCl aqueous solution, 2N NaOH aqueous solution and brine, dried over MgSO₄ and concentrated in vacuo. Purification of the residue by flash chromatography on silica gel (isohexane/AcOEt: 3/1) gave 3[methanesulfonyl-(4-trifluoromethyl-phenyl)-amino]-5 benzoic acid ethyl ester (C27) (450 mg, 60%) as a colourless oil.

The following esters were obtained in analogous manner to that described for Ester 27 from the appropriate diphenylamine using the appropriate alkylsulfonyl chloride:

| Ester | N,N-diarylamine |
|---|---|
| 3-(Ethanesulfonyl-phenyl-amino)-benzoic acid methyl | D49 |
| ester (C28) | |
| 3-[(Butane-1-sulfonyl)-phenyl-amino]-benzoic acid | D49 |
| methyl ester (C29) | |
| 3-[Phenl-(propane-2-sulfonyl)-amino]-benzoic acid | D49 |
| methyl ester (C30) | |
| 2-Fluoro-3-(methanesulfonyl-phenyl-amino)-benzoic | D31 |
| acid tert-butyl ester (C31) | |
| 4-Fluoro-3-(methanesulfonyl-phenyl-amino)-benzoic | D32 |
| acid tert-butyl ester (C32) | |
| 3-[Methanesulfonyl-(2-methoxy-phenyl)-amino]- | D33 |
| benzoic acid methyl ester (C33) | |
| 3-[methanesulfonyl-(3-meethoxy-phenyl)-amino]- | D34 |
| benzoic acid methyl ester (C34) | |
| 3-[Methanesulfonyl-(4-methoxy-phenyl)-amino]- | D35 |
| benzoic acid methyl ester (C35) | |
| 3-[Methanesulfonyl-(2-chloro-phenyl.)-amino]- | D36 |
| benzoic acid methyl ester (C36) | |
| 3-[Methanesulfonyl-(3-chloro-phenyl)-amino]- | D37 |
| benzoic acid methyl ester (C37) | |
| 3-[Methanesulfonyl-(4-chloro-phenyl)-amino]- | D38 |
| benzoic acid methyl ester (C38) | |
| 3-[Methanesulfonyl-(3,4-dichloro-phenyl)-amino]- | D39 |
| benzoic acid methyl ester (C39) | |
| 3-[Methanesulfonyl-(3,5-dichloro-phenyl)-amino]- | D40 |
| benzoic acid methyl ester (C40) | |
| 3-[Methanesulfonyl-(2-cyano-phenyl)-amino]- | D41 |
| benzoic acid methyl ester (C41) | |
| 3-[Methanesulfonyl-(3-cyano-phenyl)-amino]- | D42 |
| benzoic acid methyl ester (C42) | |
| 3-[Methanesulfonyl-(4-cyano-phenyl)-amino]- | D43 |
| benzoic acid methyl ester (C43) | |
| 3-Bromo-5-(methanesulfonyl-phenyl-amino)-benzoic | D44 |
| acid dimethyl-ethyl ester (C44) | |
| 3-(Methanesulfonyl-naphthalen-1-yl-amino)-benzoic | D45 |
| acid tert-butyl ester (C45) | |
| 3-(Methanesulfonyl-phhenyl-amino)-5-nitro-benzoic | F25 |
| acid methyl ester (F26) | |

Ester 47
3-(Methanesulfonyl-pyridin-3-yl-amino)-benzoic acid tert-butyl ester (C47)

To a solution of 3-(pyridin-3-ylamino)-benzoic acid tert-butyl ester (D47) (420 mg, 1.56 mmol, 1 equiv) in THF (10 ml) at −78° C. was added LDA (2M in THF/n-heptane, 2.33 ml, 4.67 mmol, 3 equiv) dropwise and the resulting mixture was stirred at this temperature for a further 5 min. Methanesulfonyl chloride (361 μl, 4.9 mmol, 2.5 equiv) was then added dropwise and the resulting mixture was allowed to warm to room temperature over 30 min then diluted with AcOEt. The organic phase was washed with 2N NaOH aqueous solution and brine, dried over MgSO₄ and concentrated in vacuo. Purification of the residue by flash chromatography on silica gel (iso-hexane/AcOEt: 1/2) gave 3-(methanesulfonyl-pyridin-3-yl-amino)-benzoic acid tert-butyl ester (C47) (230 mg, 42%) as a pale yellow oil.

The following esters were obtained using an analogous process to that described for Ester 47 from the appropriate diphenylamine starting material:

| | Starting |
|---|---|
| Ester | Material |
| 3-(Methanesulfonyl-pyridin-2-yl-amino)-benzoic acid tert-butyl | D46 |
| ester (C46) | |
| 3-(Methanesulfonyl-pyridin-4-yl-amino)-benzoic acid tert-butyl | D48 |
| ester (C48) | |

Ester 49
3-Ethylamino-5-(methanesulfonyl-phenyl-amino)-benzoic acid methyl ester (C49)

To a solution of 3-amino-5-(methanesulfonyl-phenyl-amino)benzoic acid methyl ester (F27) (330 mg, 1.03 mmol, 1 equiv) in (CH₂Cl)₂ (10 ml) was added sodium triacetoxyborohydride (306 mg, 1.44 mmol, 1.4 equiv), acetaldehyde (86 μl, 1.44 mmol, 1.4 equiv) and CH₃COOH (71 μl, 1.24 mmol, 1.2 equiv). The resulting mixture was stirred at room temperature for 2 h, diluted with CH₂Cl₂ (20 ml), washed with saturated aqueous NaHCO₃ solution (20 ml), dried over MgSO₄ and concentrated in vacuo. Purification of the residue by flash chromatography on silica gel (iso-hexane/AcOEt: 1/2) 3-ethylamino-5-(methanesulfonyl-phenyl-amino)-benzoic acid methyl ester (C49) (260 mg, 72%) as a pale yellow solid.

Ester 50

3-(Benzyl-methyl-amino)-5-(methanesulfonyl-phenyl-amino)-benzoic acid tert-butyl ester (C50)

A flask was charged under nitrogen with 3-bromo-5-(methanesulfonyl-phenyl-amino)-benzoic acid tert-butyl ester (C44) (800 mg, 1.9 mmol, 1 equiv), sodium tert-butoxide (274 mg, 2.85 mmol, 1.5 equiv), tris(dibenzylideneacetone)dipalladium(0) (92 mg, 0.1 mmol, 0.05 equiv), 2-(dicyclohexylphosphino)biphenyl (50 mg, 0.14 mmol, 0.075 equiv) and toluene (20 ml). N-Methylbenzylamine (368 μl, 2.85 mmol, 1.5 equiv) was then added via syringe and the resulting mixture was stirred at 90° C. for 2 h then N-methylbenzylamine (200 μl, 1.54 mmol, 0.8 equiv) was added. After 3 h, the mixture was cooled to room temperature, diluted with H₂O and AcOEt. The layers were separated, the aqueous phase diluted with saturated aqueous NaHCO₃ solution and extracted with AcOEt. The combined organic phases were dried over MgSO₄ and concentrated in vacuo. Purification of the residue by flash chromatography on silica gel (iso-hexane/AcOEt: 3/1) gave 3-(benzyl-methyl-amino)-5-(methanesulfonyl-phenyl-amino)-benzoic acid tert-butyl ester (C50) (600 mg, 68%) as a pale yellow oil.

Ester 51

3-(Methanesulfonyl-phenyl-amino)-5-(2-oxo-pyrrolidin-1-yl)-benzoic acid tert-butyl ester (C51)

A flask was charged under nitrogen with 3-bromo-5-(methanesulfonyl-phenyl-amino)-benzoic acid tert-butyl ester (C44) (1.1 g, 2.58 mmol, 1 equiv), Cs₂CO₃ (1.18 g, 3.61 mmol, 1.4 equiv), tris(dibenzylideneacetone)dipalladium(0) (47 mg, 0.05 mmol, 0.02 equiv), Xantphos (90 mg, 0.15 mmol, 0.06 equiv) and toluene (20 ml). Pyrrolidin-2-one (216 μl, 2.84 mmol, 1.1 equiv) was then added via syringe and the resulting mixture was stirred at 100° C. for 16 h. Xantphos (30 mg, 0.05 mmol, 0.02 equiv), tris(dibenzylideneacetone)dipalladium(0) (20 mg, 0.021 mmol, 0.085 equiv) and pyrrolidin-2-one (100 μl, 1.31 mmol, 0.5 equiv) were then added and the stirring was continued at 100° C. for 16 h. The mixture was then cooled to room temperature and concentrated in vacuo. The residue was partitioned between H₂O and AcOEt and the aqueous phase re-extracted with AcOEt. The combined organic solutions were dried over MgSO₄ and concentrated in vacuo. Purification of the residue by flash chromatography on silica gel (iso-hexane/AcOEt: 3/2) gave 3-(methanesulfonyl-phenyl-amino)-5-(2-oxo-pyrrolidin-1-yl)-benzoic acid tert-butyl ester (C51) (1.0 g, 90%) as a white solid.

Ester 53

3-Ethynyl-5-(methanesulfonyl-phenyl-amino)-benzoic acid tert-butyl ester (C53)

To a solution of 3-(hydroxy-methyl-but-1-ynyl)-5-(methanesulfonyl-phenyl-amino) benzoic acid tert-butyl ester (F15) (600 mg, 1.40 mmol, 1 equiv) in toluene (20 ml) was added NaH (60% dispersion in mineral oil, 60 mg, 1.5 mmol, 1.1 equiv). The resulting mixture was stirred at 110° C. for 2 h, cooled to room temperature and diluted with AcOEt. The organic phase was washed with saturated aqueous NaHCO₃ solution, dried over MgSO₄ and concentrated in vacuo to give 3-ethynyl-5-(methanesulfonyl-phenyl-amino) benzoic acid tert-butyl ester (C53) (550 mg, 105%) as a pale brown oil.

Ester 54

(Methanesulfonyl-phenyl-amino)-methyl-biphenyl-3-carboxylic acid dimethyl-ethyl ester (C54)

Ester 54 was prepared according to an analogous procedure described for Ester 55, using (2-methylphenyl)boronic acid instead of 2,6-dimethylphenylboronic acid which yielded (methanesulfonyl-phenyl-amino)-methyl-biphenyl-3-carboxylic acid dimethyl-ethyl ester (C54) (130 mg, 63%) from 3-bromo-5-(methanesulfonyl-phenyl-amino)benzoic acid tert-butyl ester (C44) (200 mg, 0.47 mmol).

Ester 55

(Methanesulfonyl-phenyl-amino)-dimethyl-biphenyl-3-carboxylic acid tert-butyl ester (C55)

To a solution of 3-bromo-5-(methanesulfonyl-phenyl-amino)-benzoic acid tert-butyl ester (C44) (200 mg, 0.47 mmol, 1 equiv) in toluene (1 ml) and EtOH (1 ml) were added tetrakis(triphenylphosphine)-palladium(0) (54 mg, 0.047 mmol, 0.1 equiv), 2,6-dimethylphenylboronic acid (74 mg, 0.49 mmol, 1.05 equiv) and K₂CO₃ (98 mg, 0.71 mmol, 1.5 equiv) and the resulting mixture was stirred at 90° C. for 16 h, cooled to room temperature and diluted with AcOEt. The organic phase was washed with a 2N NaOH aqueous solution, dried over MgSO₄ and concentrated in vacuo. Purification by flash chromatography on silica gel (iso-hexane/AcOEt: 3/1) gave (methanesulfonyl-phenyl-amino)-dimethyl-biphenyl-3-carboxylic acid tert-butyl ester (C55) (130 mg, 61%) as a pale yellow oil.

Ester 56

3-Cyclopentyl-5-(methanesulfonyl-phenyl-amino)-benzoic acid tert-butyl ester (C56)

To a solution of 3-cyclopent-1-enyl-5-(methanesulfonyl-phenyl-amino)benzoic tert-butyl ester, 3-cyclopent-2-enyl-5-(methanesulfonyl-phenyl-amino)-benzoic acid tert-butyl ester and 3-cyclopent-3-enyl-5-(methanesulfonyl-phenyl-amino)-benzoic acid tert-butyl ester (F12) (450 mg, 1.09 mmol, 1 equiv) in EtOH (10 ml) and H₂O (2 ml) was added 10% palladium on charcoal (50% wet, 450 mg, 5% w/w) and NH₄COOH (687 mg, 10.90 mmol, 10 equiv). The resulting mixture was stirred at 50° C. for 1 h then cooled to room temperature. The catalyst was removed by filtration over a pad of celite. Most of the EtOH was removed in vacuo and the residue dissolved in AcOEt. The organic phase was washed with H₂O, dried over MgSO₄ and concentrated in vacuo to give 3 cyclopentyl-5-(methanesulfonyl-phenyl-amino)-benzoic acid tert-butyl ester (C56) (420 mg, 92%) as a colorless oil.

The following esters were prepared in an analogous manner to the procedure described for Ester 56:

| | Starting |
|---|---|
| Ester | Material |
| 3-Cyclohexyl-5-(methanesulfonyl-phenyl-amino)-benzoic acid | F13 |
| tert-butyl ester (C57) | |
| 3-Isobutyl-5-(methanesulfonyl-phenyl-amino)-benzoic acid tert- | F14 |
| butyl ester (C52) | |

Ester 58
3-(Methanesulfonyl-phenyl-amino)-5-prop-1-ynyl-benzoic acid tert-butyl ester (C58)

To a solution of 3-bromo-5-(methanesulfonyl-phenyl-amino)-benzoic acid tert-butyl ester (C44) (200 g, 0.47 mmol, 1 equiv) in toluene (10 ml) was added tetrakis(triphenylphosphine)-palladium(0) (16 mg, 0.014 mmol, 0.03 equiv) and tributyl-prop-1-ynyl-stannane (171 μl, 0.56 mmol, 1.2 equiv) and the resulting mixture was stirred at 100° C. for 16 h then cooled to room temperature and concentrated in vacuo. Purification by flash chromatography on silica gel (iso-hexane/AcOEt: 17/3) gave 3-(ethanesulfonyl-phenyl-amino)-5-prop-1-ynyl-benzoic acid tert-butyl ester (C58) (150 mg, 83%) as a colourless oil.

Preparation of BOC-Protected Amines

Description G1

((1S,2R)-1-Benzyl-3-cyclohexylamino-2-hydroxy-propyl)-carbamic acid tert-butyl ester (G1)

((S)—(S)-1-Oxiranyl-2-phenyl-ethyl)-carbamic acid tert-butyl ester (10 g, 38 mmol, 1 equiv) [Chirex 1819W94 Lot#9924382] was dissolved in EtOH (100 ml) and cyclohexylamine (13 ml, 114 mmol, 3 equiv) was added. The resulting mixture was heated, under an atmosphere of nitrogen, for 12 h at reflux temperature. The mixture was cooled and the solvent was removed by evaporation in vacuo. The resulting white solid was washed with H₂O and then with Et₂O before drying in vacuo to give ((1S,2R)-1-benzyl-3-cyclohexylamino-2-hydroxy-propyl)carbamic acid tert-butyl ester (G1) (9.0 g, 66%).

[M+H]⁺=363.2

The following Boc-protected amines were prepared in an analogous manner to that described for Description G1 by substituting cyclohexylamine with the starting materials indicated:

| | Starting |
|---|---|
| Boc-protected Amine | Material |
| [(1S,2R)-1-Benzyl-3-((S)-1-cyclo- | F6 |
| hexylcarbamoyl-ethylamino)-2- | |
| hydroxy-propyl]- | |
| tert-butyl ester (G2) | |
| [(1S,2R)-1-Benzyl-3-(1,5- dimethyl-hexylamino)-2-hydroxy- propyl]-carbamic acid tert- butyl ester (G3) | 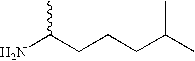 |
| [(1S,2R)-1-Benzyl-2-hydroxy- 3-(3-methoxy-benzylamino) propyl]-carbamic acid tert- butyl ester (G4) | 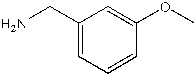 |
| [(1S,2R)-1-Benzyl-2-hydroxy- 3-(3-trifluoromethoxy- benzylamino)-propyl]-carbamic acid tert-butyl ester (G5) | 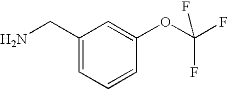 |
| [(1S,2R)-1-Benzyl-2-hydroxy- 3-(3-trifluoromethyl- benzylamino)-propyl]-carbamic acid tert-butyl ester (G6) | 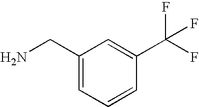 |
| {(1S,2R)-1-Benzyl-2-hydroxy- | F5 |
| 3-[1-(3-methoxy-phenyl)-1- | |
| methyl-ethylamino]-propyl}- | |
| carbamic acid tert-butyl ester (G7) | |
| ((1S,2R)-1-Benzyl-3-cyclo- propylamino-2-hydroxy-propyl)- carbamic acid tert-butyl ester (G8) | 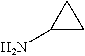 |
| ((1S,2R)-1-Benzyl-3-[(1-ethyl- 1H-pyrazol-4-ylmethyl]- 2-hydroxy-propyl)-carbamic acid tert-butyl ester (G9) | 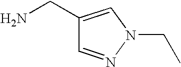 |
| [(1S,2R)-1-Benzyl-2-hydroxy-3- | F51 |
| (1,1,5-trimethyl-hexylamino)- | |
| propyl]-carbamic acid | |
| tert-butyl ester (G10) | |

Preparation of Amines
Amine 1
(2R,3S)-3-Amino-1-cyclohexylamino-4-phenyl-butan-2-ol di-hydrogen chloride (B1)

((1S,2R)-1-benzyl-3-cyclohexylamino-2-hydroxy-propyl)-carbamic acid tert-butyl ester (G1) (9 g, 25 mmol, 1 equiv) was dissolved in MeOH (70 ml) and then a 4M solution of HCl in dioxan (60 ml, excess) was added. The resulting mixture was stirred for 3 h at room temperature and then the solvents were removed by evaporation in vacuo. The resulting residue was washed with AcOEt and then with Et₂O before drying in vacuo to give (2R,3S)-3-amino-1-cyclohexylamino-4-phenyl-butan-2-ol di-hydrogen chloride (B1) as a white solid (7.4 g, 88%).

The following amines were prepared in an analogous manner described for Amine 1 substituting the appropriate BOC-protected amines for ((1S,2R)-1-benzyl-3-cyclohexylamino-2-hydroxy-propyl)carbamic acid tert-butyl ester. In some cases the 4M HCl in dioxane was replaced with 3 equivalents of p-toluene sulphonic acid to give the tosic acid salts as the product.

| | Starting |
|---|---|
| Amine | Material |
| (S)-2-((2R,3S)-3-Amino-2-hydroxy-4-phenyl-butylamino)-N- | G2 |
| cyclohexyl-propionamide di-hydrogen chloride (B2) | |
| (2R,3S)-3-Amino-1-(1,5-dimethyl-hexylamino)-4-phenyl-butan- | G3 |
| 2-ol di-hydrogen chloride (B3) | |
| (2R,3S)-3-Amino-1-(3-methoxy-benzylamino)-4-phenyl-butan-2- | G4 |
| ol (B4) | |
| (2R,3S)-3-Amino-4-phenyl-1-(3-trifluoromethoxy-benzylamino)- | G5 |
| butan-2-ol ditosylate (B5) | |
| (2R,3S)-3-Amino-4-phenyl-1-(3-trifluoromethyl-benzylamino)- | G6 |
| butan-2-ol ditosylate (B6) | |
| (2R,3S)-3-Amino-1-[1-(3-methoxy-phenyl)-1-methyl- | G7 |
| ethylamino]-4-phenyl-butan-2-ol di tosylate (B7) | |
| (2R,3S)-3-Amino-1-sec-butylamino-4-phenyl-butan-2-ol di- | G8 |
| hydrogen chloride (B8) | |
| (2R,3S)-3-Amino-1-[(1-ethyl-1H-pyrazol-4-ylmethyl)-amino]-4- | G9 |
| phenyl-butane-2-ol di tosylate (B9) | |
| (2R,3S)-3-Amino-4-phenyl-1-(1,1,5-trimethyl-hexylamino)- | G10 |
| butan-2-ol di tosylate (B10) | |

Preparation of Acids
Acid 1 (A1)
3-(Methanesulfonyl-phenyl-amino)-benzoic acid (A1)

To a solution 3-(methanesulfonyl-phenyl-amino)-benzoic acid methyl ester (C1) (680 mg, 2.23 mmol, 1 equiv) in MeOH (5 ml) was added 2N aqueous NaOH solution (5 ml, 10 mmol, 4.5 equiv). The resulting mixture was stirred for 2 h then most of MeOH was removed in vacuo. The residue was diluted with H₂O and extracted with Et₂O. The aqueous layer was acidified using 2N aqueous HCl solution and the white precipitate formed was extracted twice with AcOEL The combined organic solutions were dried over MgSO₄ and concentrated in vacuo to give 3-(methanesulfonyl-phenyl-amino)benzoic acid (A1) (600 mg, 92%) as a white solid, which was used in the next step without further purification.

The following Acids were prepared in an analogous manner to that described for Acid 1 from the appropriate starting material:

| | Starting |
|---|---|
| Acid | Material |
| 3-(Dioxo-1/⁶-isothiazolidin-2-yl)-5-(methanesulfonyl-phenyl- | C2 |
| amino)-benzoic acid (A2) | |
| 3-(Methanesulfonyl-phenyl-amino)-5-N,N-dipropyl- | C3 |
| isophthalamic acid (A3) | |
| 3-Ethoxy-5-(methanesulfonyl-phenyl-amino)-benzoic acid (A4) | C4 |
| 3-(Methanesulfonyl-naphthalen-2-yl-amino)-benzoic acid (A5) | C5 |
| Cyano-(methanesulfonyl-phenyl-amino)-benzoic acid (A7) | C7 |
| 3-(Methanesulfonyl-phenyl-amino)-2-methyl-benzoic acid (A10) | C10 |
| 3-(Methanesulfonyl-phenyl-amino)-4-methyl-benzoic acid (A11) | C11 |
| 4-Chloro-3-(methanesulfonyl-phenyl-amino)-benzoic acid (A12) | C12 |
| 3-(Methanesulfonyl-phenyl-amino)-5-methyl-benzoic acid (A13) | C13 |
| 3-(Methanesulfonyl-phenyl-amino)-5-methylsulfanyl-benzoic | C14 |
| acid (A14) | |
| 3-(Methanesulfonyl-phenyl-amino)-5-ethylsulfanyl-benzoic | C15 |
| acid (A15) | |

Acid 16
5-(Methanesulfonyl-phenyl-amino)-nicotinic acid (A16)

To a solution 5-(methanesulfonyl-phenyl-amino)-nicotinic acid ethyl ester (C16) (514 mg, 1.6 mmol, 1 equiv) in EtOH (50 ml) was added 2N aqueous NaOH solution (20 ml, 40 mmol, excess). The resulting mixture was stirred for 16 h then most of EtOH was removed in vacuo. The residue was partitioned between AcOEt and 5% citric acid aqueous solution (pH 4) and the two layers were separated. The aqueous phase was extracted with AcOEt and the combined organic solutions were dried over MgSO₄ then concentrated in vacuo to give 5-(methanesulfonyl-phenyl-amino)-nicotinic acid (A16) (130 mg, 28%) as a cream solid, which was used in the next step without further purification.

The following Acid was prepared in an analogous manner to that described for Acid 16 from the appropriate starting material:

| | Starting |
|---|---|
| Acid | Material |
| 2-(Methanesulfonyl-phenyl-amino)-isonicotinic acid (A17) | C17 |

The following Acids were prepared in an analogous manner to that described for Acid 1 from the appropriate starting material:

| Acid | Starting Material |
|---|---|
| 3-[(4-Fluoro-phenyl)-methanesulfonyl-amino]-5-(2-oxo-pyrrolidin-1- | C18 |
| yl)-benzoic acid (A18) | |
| 3-[(3-Fluoro-phenyl)-methanesulfonyl-amino]-5-(2-oxo-pyrrolidin-1- | C19 |
| yl)-benzoic acid (A19) | |
| 3-[(3-Methoxy-phenyl)-methanesulfonyl-amino]-5-(2-oxo-pyrrolidin- | C20 |
| 1-yl)-benzoic acid (A20) | |
| 3-[(4-Methoxy-phenyl)-methanesulfonyl-amino]-5-(2-oxo-pyrrolidin- | C21 |
| 1-yl)-benzoic acid (A21) | |
| 3-[(3,4-Difluoro-phenyl)-methanesulfonyl-amino]-5-(2-oxo- | C22 |
| pyrrolidin-1-yl)-benzoic acid (A22) | |
| 3-[(3,5-Difluoro-phenyl)-methanesulfonyl-amino]-5-(2-oxo- | C23 |
| pyrrolidin-1-yl)-benzoic acid (A23) | |
| 3-[(3-Cyano-phenyl)-methanesulfonyl-amino]-5-(2-oxo-pyrrolidin-1- | C24 |
| yl)-benzoic acid (A24) | |
| 3-[(4-Cyano-phenyl)-methanesulfonyl-amino]-5-(2-oxo-pyrrolidin-1- | C25 |
| yl)-benzoic acid (A25) | |
| 3-[(3-Carbamoyl-phenyl)-methanesulfonyl-amino]-5-(2-oxo- | C26 |
| pyrrolidin-1-yl)-benzoic acid (A26) | |
| 3-[Methanesulfonyl-(4-trifluoromethyl-phenyl)-amino]-benzoic acid | C27 |
| (A27) | |
| 3-(Ethanesulfonyl-phenyl-amino)-benzoic acid (A28) | C28 |
| 3-[(Butane-1-sulfonyl)-phenyl-amino]-benzoic acid (A29) | C29 |
| 3-[Phenyl-(propane-2-sulfonyl)-amino]-benzoic acid (A30) | C30 |
| 2-Fluoro-3-(methanesulfonyl-phenyl-amino)-benzoic acid (A31) | C31 |
| 3-[Methanesulfonyl-(2-methoxy-phenyl)-amino]-benzoic acid (A33) | C33 |
| 3-[Methanesulfonyl-(3-methoxy-phenyl)-amino]-benzoic acid (A34) | C34 |
| 3-[Methanesulfonyl-(4-methoxy-phenyl)-amino]-benzoic acid (A35) | C35 |
| 3-[Methanesulfonyl-(2-chloro-phenyl)-amino]-benzoic acid (A36) | C36 |
| 3-[Methanesulfonyl-(3-chloro-phenyl)-amino]-benzoic acid (A37) | C37 |
| 3-[Methanesulfonyl-(4-chloro-phenyl)-amino]-benzoic acid (A38) | C38 |
| 3-[Methanesulfonyl-(3,4-dichloro-phenyl)-amino]-benzoic acid | C39 |
| (A39) | |
| 3-[Methanesulfonyl-(3,5-dichloro-phenyl)-amino]-benzoic acid | C40 |
| (A40) | |
| 3-[Methanesulfonyl-(2-cyano-phenyl)-amino]-benzoic acid (A41) | C41 |
| 3-[Methanesulfonyl-(3-cyano-phenyl)-amino]-benzoic acid (A42) | C42 |
| 3-[Methanesulfonyl-(4-cyano-phenyl)-amino]-benzoic acid (A43) | C43 |
| 3-Ethylamino-5-(methanesulfonyl-phenyl-amino)-benzoic acid | C49 |
| (A49) | |
| 3-(Methanesulfonyl-phenyl-amino)-5-(2-oxo-pyrrolidin-1-yl)-benzoic | C51 |
| acid (A51) | |
| Ethyl-(methanesulfonyl-phenyl-amino)1H-indole-5-carboxylic acid | C59 |
| (A61) | |

Acid 44
3-Bromo-5-(methanesulfonyl-phenyl-amino)-benzoic acid (A44)

A solution of 3-bromo-5-(methanesulfonyl-phenyl-amino)benzoic acid tert-butyl ester (C44) (2 g, 4.7 mmol, 1 equiv) in CH₂Cl₂/CF₃COOH (1/1, 30 ml) was stirred at room temperature for 2 h then concentrated in vacuo. Traces of solvent were removed by azeotroping with toluene. The residue was triturated with Et₂O/iso-hexane (1:1) and filtered off to give 3-bromo-5-(methanesulfonyl-phenyl-amino)-benzoic acid (A44) (1.45 mg, 83%) as a white solid which was used in the next step without further purification.

The following Acids were prepared in an analogous manner to that described for Acid 44 from the appropriate starting material:

| | Starting |
|---|---|
| Acid | Material |
| 3-[(4-Fluoro-phenyl)-methanesulfonyl-amino]-benzoic acid (A6) | C6 |
| 3-[(3-Fluoro-phenyl)-methanesulfonyl-amino]-benzoic acid (A8) | C8 |
| 3-[(3,5-Difluoro-phenyl)-methanesulfonyl-amino]-benzoic acid | C9 |
| (A9) | |
| 4-Fluoro-3-(methanesulfonyl-phenyl-amino)-benzoic acid (A32) | C32 |
| 3-(Methanesulfonyl-naphthalen-1-yl-amino)-benzoic acid (A45) | C45 |
| 3-(Methanesulfonyl-pyridin-2-yl-amino)-benzoic acid (A46) | C46 |
| 3-(Methanesulfonyl-pyridin-3-yl-amino)-benzoic acid (A47) | C47 |
| 3-(Methanesulfonyl-pyridin-4-yl-amino)-benzoic acid (A48) | C48 |
| 3-(Benzyl-methyl-amino)-5-(methanesulfonyl-phenyl-amino)- | C50 |
| benzoic acid (A50) | |
| 3-Isobutyl-5-(methanesulfonyl-phenyl-amino)-benzoic acid | C52 |
| (A52) | |
| 3-Ethynyl-5-(methanesulfonyl-phenyl-amino)-benzoic acid | C53 |
| (A53) | |
| (Methanesulfonyl-phenyl-amino)-methyl-biphenyl-3- | C54 |
| carboxylic acid (A54) | |
| (Methanesulfonyl-phenyl-amino)-dimethyl-biphenyl-3- | C55 |
| carboxylic acid (A55) | |
| 3-Cyclopentyl-5-(methanesulfonyl-phenyl-amino)-benzoic acid | C56 |
| (A56) | |
| 3-Cyclohexyl-5-(methanesulfonyl-phenyl-amino)-benzoic acid | C57 |
| (A57) | |
| 3-(Methanesulfonyl-phenyl-amino)-5-(prop-1-ynyl)-benzoic acid | C58 |
| (A58) | |

Acid 59
3-Methanesulfonyl-5-(methanesulfonyl-phenyl-amino)-benzoic acid (A59)

To a solution of 3-(methanesulfonyl-phenyl-amino)-5-methylsulfanyl-benzoic acid (A14) (67 mg, 0.199 mmol, 1 equiv) in MeOH/H₂O (3:1, 12 ml) was added oxone (488 mg, 0.79 mmol, 4 equiv). The resulting mixture was stirred at room temperature for 2 h and then concentrated in vacuo. The residue was partitioned between AcOEt and H₂O and the layers separated. The organic layer was washed with H₂O and brine, dried over Na₂SO₄ and concentrated in vacuo to give a solid which was triturated with Et₂O to give 3-methanesulfonyl-5-(methanesulfonyl-phenyl-amino)benzoic acid (A59) (66 mg, 90%) as a white solid. [M−H]⁻=368.0, RT=2.64 min

Acid 60

3-Ethanesulfonyl-5-(methanesulfonyl-phenyl-amino)-benzoic acid (A60)

A60 was prepared in an analogous manner to that described for Acid 59 from 53 mg (0.15 mmol) of 3-(methanesulfonyl-phenyl-amino)-5-ethylsulfanyl-benzoic acid (A15) which yielded 56 mg (96%) of 3-ethanesulfonyl-5-(methanesulfonyl-phenyl-amino)-benzoic acid (A60) as a white solid. [M−H]⁻=382.0, RT=2.73 min

### Preparation of Examples

### Example 1

N-[(1S,2R)-1-Benzyl-3-((S)-1-cyclohexylcarbamoyl-ethylamino)-2-hydroxy-propyl]-(methanesulfonyl-phenyl-amino)-benzamide (E1)

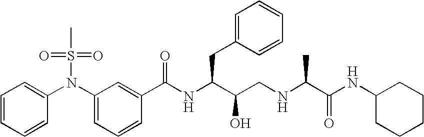

To a solution of 3-(methanesulfonyl-phenyl-amino)-benzoic acid (A1) (45 mg, 0.15 mmol, 1 equiv) in DMF (5 ml) at room temperature was added (S)-2-((2R,3S)-3-amino-2-hydroxy-4-phenyl-butylamino)- N-cyclohexyl-propionamide di-hydrogen chloride (B2) (63 mg, 0.15 mmol, 1 equiv), 1-(3-dimethylaminopropyl)-3-ethyl-carbodiimide hydrochloride (28 mg, 0.15 mmol, 1.2 equiv), 1-hydroxybenzotriazole hydrate (23 mg, 0.15 mmol, 1.2 equiv) and 4-ethylmorpholine (120 μl, 0.93 mmol, 6 equiv). The resulting mixture was stirred for 4 h then concentrated in vacuo. The residue was diluted with AcOEt and the organic phase washed with saturated aqueous NaHCO₃ solution, dried over MgSO₄ and concentrated in vacuo. The residue was purified by trituration with Et₂O to yield N-[(1S,2R)-1-benzyl-3-((S)-1-cyclohexylcarbamoyl-ethylamino)-2-hydroxy-propyl]-(methanesulfonyl-phenyl-amino)benzamide (E1) as a white solid (46 mg, 50%).

[M+H]⁺=607.2

RT=2.63 min

### Examples 2-79 (E2-E79)

Examples 2-79 were prepared in an analogous manner to Example 1 from the appropriate acid and amines indicated in the below table:

| | | | | | RT |
|---|---|---|---|---|---|
| Example | Structure | Acid | Amine | [M + H]⁺ | (min) |
| N-[(1S,2R)-1-Benzyl-3-((S)-1- cyclohexylcarbamoyl-ethylamino)- 2-hydroxy-propyl]-(ethanesulfonyl- phenyl-amino)-benzamide (E2) | 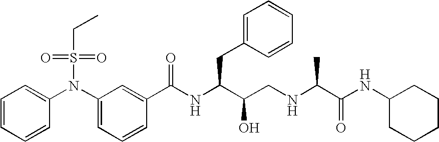 | A28 | B2 | 621.2 | 2.71 |
| N-[(1S,2R)-1-Benzyl-3-((S)-1- cyclohexylcarbamoyl-ethylamino)- 2-hydroxy-propyl]-[(butane-1- sulfonyl)-phenyl-amino]- benzamide (E3) | 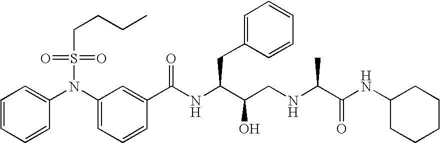 | A29 | B2 | 649.2 | 2.90 |
| N-[(1S,2R)-1-Benzyl-3-((S)-1- cyclohexylcarbamoyl-ethylamino)- 2-hydroxy-propyl]-[phenyl- (propane-2-sulfonyl-phenyl-amino]- benzamide (E4) | 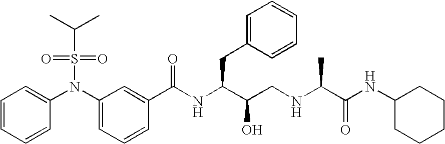 | A30 | B2 | 635.2 | 2.75 |
| N-[(1S,2R)-1-Benzyl-3-((S)-1- cyclohexylcarbamoyl-ethylamino)- 2-hydroxy-propyl]-[methanesulfonyl- (4-trifluoromethyl-phenyl)- amino]-benzamide (E5) | 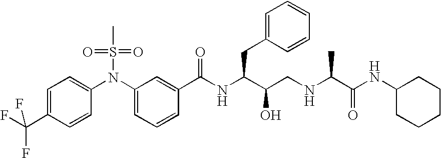 | A27 | B2 | 675.3 | 2.84 |
| N-[(1S,2R)-1-Benzyl-3-((S)-1- cyclohexylcarbamoyl-ethylamino)- 2-hydroxy-propyl]-[methanesulfonyl- (3-methoxy-phenyl)-amino]- benzamide (E6) | 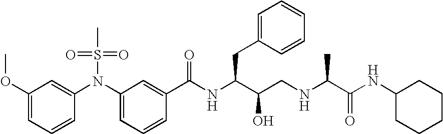 | A34 | B2 | 637.3 | 2.68 |
| N-[(1S,2R)-1-Benzyl-3-((S)-1- cyclohexylcarbamoyl-ethylamino)- 2-hydroxy-propyl]-[methanesulfonyl- (4-methoxy-phenyl)-amino]- benzamide (E7) | 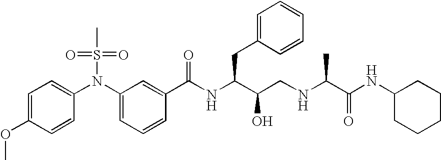 | A35 | B2 | 637.2 | 2.66 |
| N-[(1S,2R)-1-Benzyl-3-((S)-1- cyclohexylcarbamoyl-ethylamino)- 2-hydroxy-propyl]-[methanesulfonyl- (2-methoxy-phenyl)-amino]- benzamide (E8) | 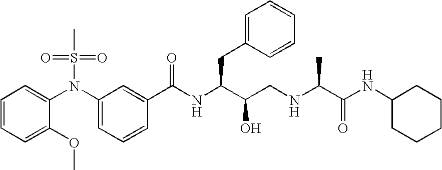 | A33 | B2 | 637.3 | 2.67 |
| N-[(1S,2R)-1-Benzyl-3-((S)-1- cyclohexylcarbamoyl-ethylamino)- 2-hydroxy-propyl]-[(3,4-dichloro- phenyl)-methanesulfonyl-amino]- benzamide (E9) | 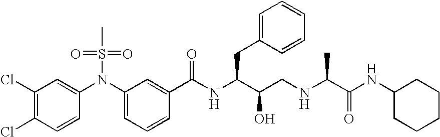 | A39 | B2 | 675.2 | 2.90 |
| N-[(1S,2R)-1-Benzyl-3-((S)-1- cyclohexylcarbamoyl-ethylamino)- 2-hydroxy-propyl]-[(3,5-dichloro- phenyl)-methanesulfonyl-amino]- benzamide (E10) | 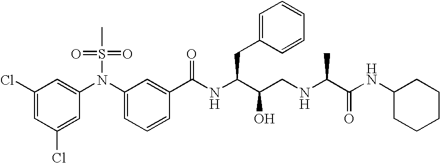 | A40 | B2 | 675.2 | 2.92 |
| N-[(1S,2R)-1-Benzyl-3-((S)-1- cyclohexylcarbamoyl-ethylamino)- 2-hydroxy-propyl]-[(2-cyano-phenyl)- methanesulfonyl-amino]- benzamide (E11) | 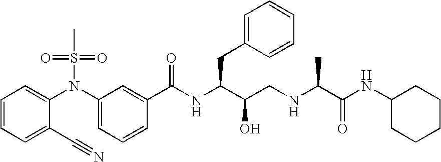 | A41 | B2 | 632.2 | 2.65 |
| N-[(1S,2R)-1-Benzyl-3-((S)-1- cyclohexylcarbamoyl-ethylamino)- 2-hydroxy-propyl]-[(4-cyano-phenyl)- methanesulfonyl-amino]- benzamide (E12) | 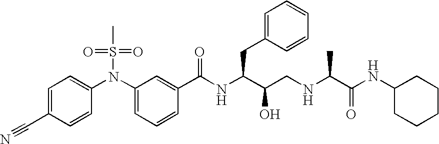 | A43 | B2 | 632.2 | 2.65 |
| N-[(1S,2R)-1-Benzyl-3-((S)-1- cyclohexylcarbamoyl-ethylamino)- 2-hydroxy-propyl]-[(2-chloro- phenyl)-methanesulfonyl-amino]- benzamide (E13) | 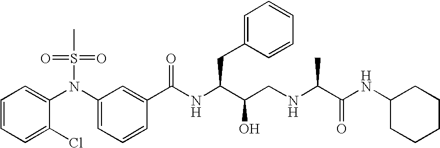 | A36 | B2 | 641.2 | 2.71 |
| N-[(1S,2R)-1-Benzyl-3-((S)-1- cyclohexylcarbamoyl-ethylamino)- 2-hydroxy-propyl]-[(4-chloro- phenyl)-methanesulfonyl-amino]- benzamide (E14) | 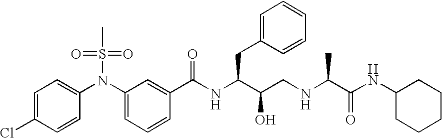 | A38 | B2 | 641.2 | 2.80 |
| N-[(1S,2R)-1-Benzyl-3-((S)-1- cyclohexylcarbamoyl-ethylamino)- 2-hydroxy-propyl]-[(3-cyano- phenyl)-methanesulfonyl-amino]- benzamide (E15) | 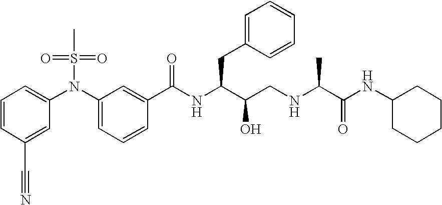 | A42 | B2 | 632.2 | 2.64 |
| N-[(1S,2R)-1-Benzyl-3-((S)-1- cyclohexylcarbamoyl-ethylamino)- 2-hydroxy-propyl]-[(3-chloro- phenyl)-methanesulfonyl-amino]- benzamide (E16) | 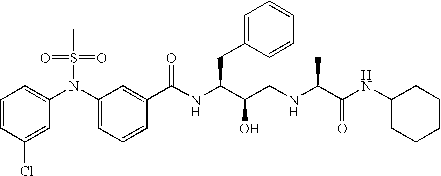 | A37 | B2 | 641.2 | 2.78 |
| N-[(1S,2R)-1-Benzyl-2-hydroxy-3- (3-trifluoromethyl-benzylamino)- propyl]-[(3-cyano-phenyl)- methanesulfonyl-amino]- benzamide (E17) | 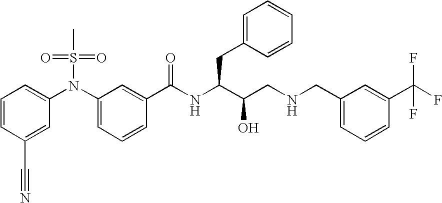 | A42 | B6 | 637.1 | 2.81 |
| N-[(1S,2R)-1-Benzyl-2-(3- trifluoromethyl-benzylamino)- propyl]-[(3-chloro-phenyl)- methanesulfonyl-amino]- benzamide (E18) | 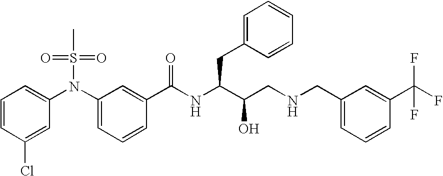 | A37 | B6 | 646.1 | 2.94 |
| N-[(1S,2R)-1-Benzyl-2-hydroxy-3- (3-trifluoromethyl-benzylamino)- propyl]-[methanesulfonyl- (3-methoxy-phenyl)-amino]- benzamide (E19) | 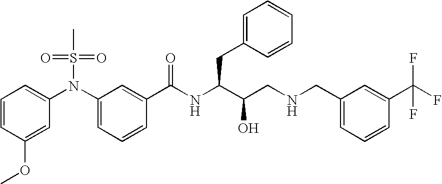 | A34 | B6 | 642.2 | 2.85 |
| N-[(1S,2R)-1-Benzyl-2-hydroxy-3- (3-trifluoromethyl-benzylamino)- propyl]-[methanesulfonyl- (4-methoxy-phenyl-amino]- benzamide (E20) | 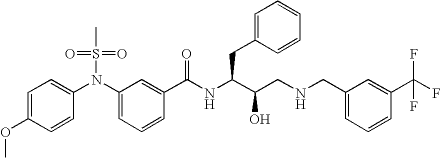 | A35 | B6 | 642.2 | 2.85 |
| N-[(1S,2R)-1-Benzyl-2-hydroxy-3- (3-trifluoromethyl-benzylamino)- propyl]-[(4- chloro-phenyl)-methanesulfonyl- amino]-benzamide (E21) | 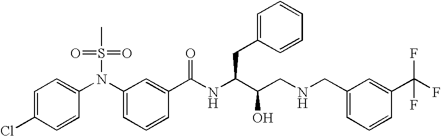 | A38 | B6 | 646.1 | 2.95 |
| N-[(1S,2R)-1-Benzyl-2-hydroxy-3- (3-trifluoromethyl-benzylamino)- propyl]-[(3,5- dichloro-phenyl)-methanesulfonyl- amino]-benzamide (E22) | 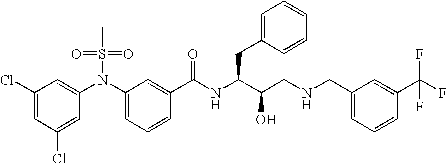 | A40 | B6 | 680.1 | 3.07 |
| N-[(1S,2R)-1-Benzyl-2-hydroxy-3- (3-trifluoromethyl-benzylamino)- propyl]-(methanesulfonyl- pyridin-3-yl-amino)- benzamide (E23) | 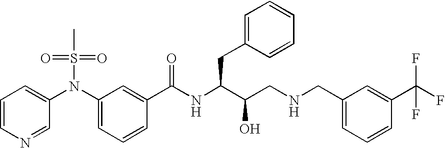 | A47 | B6 | 613.2 | 2.59 |
| N-[(1S,2R)-1-Benzyl-2-hydroxy-3- (3-trifluoromethyl-benzylamino)- propyl]-(methanesulfonyl- pyridin-2-yl-amino)- benzamide (E24) | 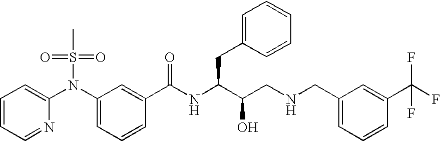 | A46 | B6 | 613.1 | 2.83 |
| N-[(1S,2R)-1-Benzyl-2-hydroxy-3- (3-trifluoromethyl-benzylamino)- propyl]-(methanesulfonyl- pyridin-4-yl-amino)- benzamide (E25) | 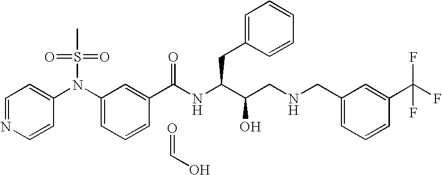 | A48 | B6 | 613.2 | 2.61 |
| N-[(1S,2R)-1-Benzyl-2-hydroxy-3- (3-trifluoromethyl-benzylamino)- propyl]-(methanesulfonyl- naphthalen-1-yl-amino)- benzamide (E26) | 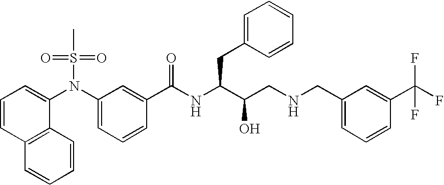 | A45 | B6 | 662.2 | 3.08 |
| N-[(1S,2R)-1-Benzyl-2-hydroxy-3- (3-trifluoromethyl-benzylamino)- propyl]-(methanesulfonyl- phenyl-amino)- benzamide (E27) | 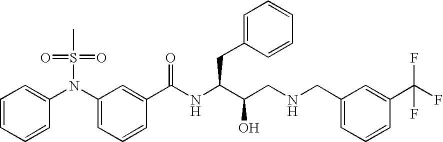 | A1 | B6 | 612.1 | 2.74 |
| N-[(1S,2R)-1-Benzyl-2-hydroxy-3- (3-trifluoromethyl-benzylamino)- propyl]-(methanesulfonyl- phenyl-amino)- benzamide (E28) | 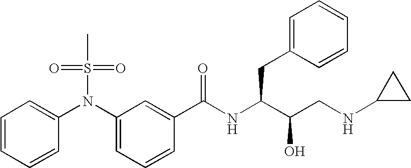 | A1 | B8 | 494.1 | 2.47 |
| N-{(1S,2R)-1-Benzyl-2-hydroxy-3- [1-(3-methoxy-phenyl)-1-methyl- ethylamino]-propyl}- benzamide formate salt (E29) | 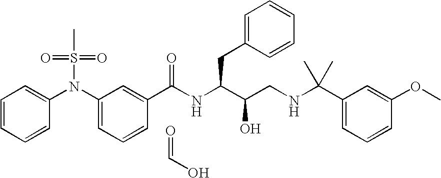 | A1 | B7 | 602.2 | 2.76 |
| N-[(1S,2R)-1-Benzyl-2-hydroxy-3- (3-trifluoromethyl-benzylamino)- propyl]-(methanesulfonyl-naphthalen- 2-yl-amino)-benzamide (E30) | 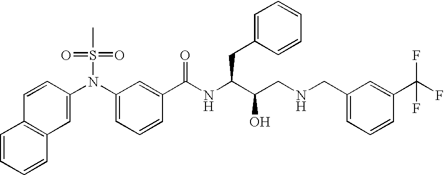 | A5 | B6 | 662.2 | 2.99 |
| N-[(1S,2R)-1-Benzyl-2-hydroxy-3- (3-trifluoromethyl-benzylamino)- propyl]-[(4-fluoro-phenyl)- methane-sulfonyl-amino]- benzamide (E31) | 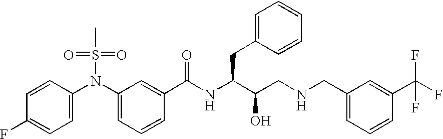 | A6 | B6 | 630.2 | 2.84 |
| N-[(1S,2R)-1-Benzyl-2-hydroxy-3- (3-trifluoromethyl-benzylamino)- propyl]-[(3-fluoro-phenyl)- methane-sulfonyl-amino]- benzamide (E32) | 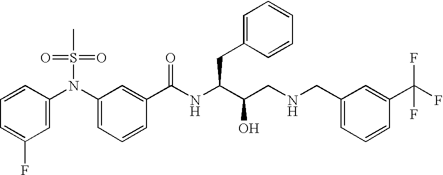 | A8 | B6 | 630.1 | 2.86 |
| N-[(1S,2R)-1-Benzyl-2-hydroxy-3- (3-trifluoromethyl-benzylamino)- propyl]-](3,5-difluoro- phenyl)-methanesulfonyl- amino]-benzamide (E33) | 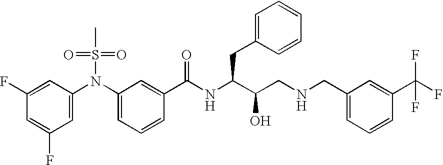 | A9 | B6 | 648.2 | 2.92 |
| N-[(1S,2R)-1-Benzyl-3-(1,5- dimethyl-hexylamino)-2-hydroxy- propyl]-(methanesulfonyl-phenyl- amino)-benzamide formate salt (E34) | 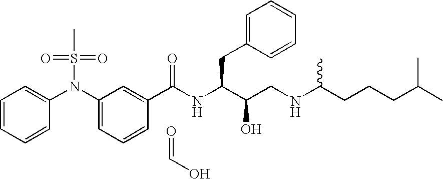 | A1 | B3 | 566.3 | 2.83 |
| N-[(1S,2R)-1-Benzyl-2-hydroxy-3- (3-methoxy-benzylamino)-propyl]- (methanesulfonyl-phenyl-amino)- benzamide (E35) | 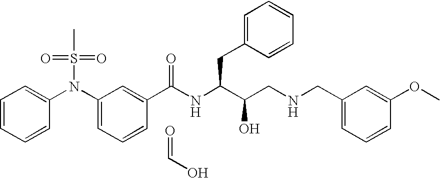 | A1 | B4 | 574.2 | 2.62 |
| N-{(1S,2R)-1-Benzyl-3-[(1-ethyl-1H- pyrazol-4-ylmethyl)amino]- 2-hydroxy-propyl}-(methanesulfonyl- phenyl-amino)-benzamide (E36) | 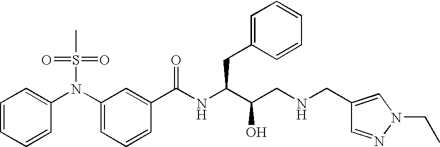 | A1 | B9 | 562.5 | 2.33 |
| N-(1S,2R)-1-Benzyl-3- cyclohexylamino-2-hydroxy-propyl)- (methanesulfonyl-phenyl-amino)- benzamide (E37) | 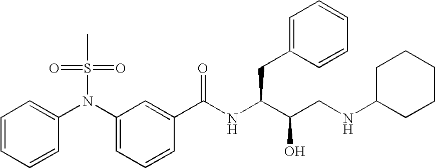 | A1 | B1 | 536.3 | 2.57 |
| N-[(1S,2R)-1-Benzyl-3-((S)-1- cyclohexylcarbamoyl-ethylamino)- 2-hydroxy-propyl]-ethylamino- (methanesulfonyl-phenyl-amino)- benzamide (E38) | 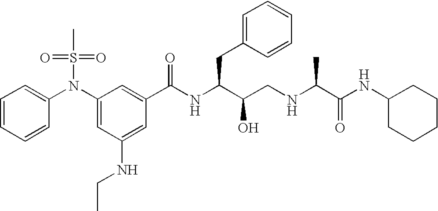 | A49 | B2 | 650.2 | 2.7 |
| N-[(1S,2R)-1-Benzyl-((S)-1- cyclohexylcarbamoyl-ethylamino)- hydroxy-propyl]-(benzyl-methyl- amino)-(methanesulfonyl-phenyl- amino)-benzamide (E39) | 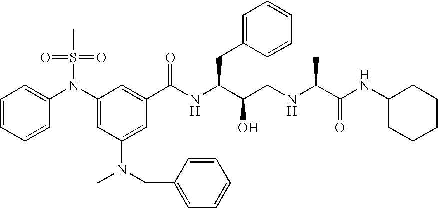 | A50 | B2 | 726.3 | 2.95 |
| N-[(1S,2R)-1-Benzyl-3- cyclohexylamino-2-hydroxy-propyl)- (methanesulfonyl-phenyl-amino)- (2-oxo-pyrrolidin-1-yl)- benzamide (E40) | 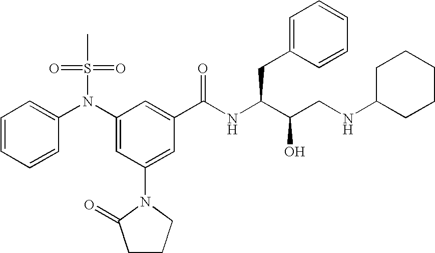 | A51 | B1 | — | 3.14 |
| N-[(1S,2R)-1-Benzyl-3-((S)-1- cyclohexylcarbamoyl-ethylamino)- 2-hydroxy-propyl]-(methanesulfonyl- phenyl-amino)-(2-oxo-pyrrolidin- 1-yl)-benzamide (E41) | 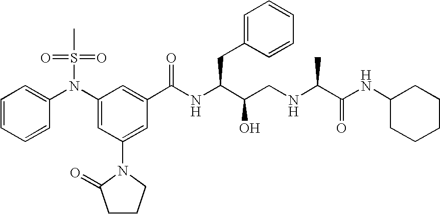 | A51 | B2 | 690.2 | 2.61 |
| N-[(1S,2R)-1-Benzyl-2-hydroxy-3- (3-methoxy-benzylamino)-propyl]- (methanesulfonyl-phenyl-amino)- benzamide (E42) | 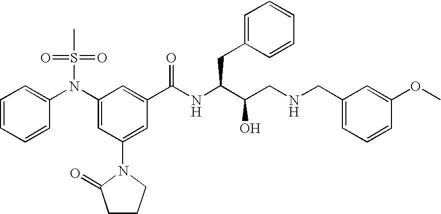 | A51 | B4 | 657.3 | 2.59 |
| N-[(1S,2R)-1-Benzyl-3-(1,5- dimethyl-hexylamino)-2-hydroxy- propyl]-(methanesulfonyl-phenyl- amino)-(2-oxo-pyrrolidin-1-yl)- benzamide (E43) | 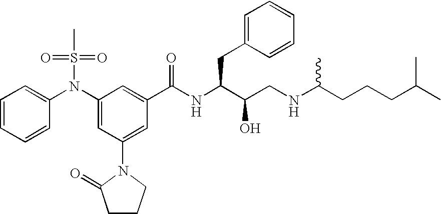 | A51 | B3 | 649.3 | 2.81 |
| N-[(1S,2R)-1-Benzyl-2-hydroxy-3- (3-trifluoromethyl-benzylamino)- propyl]-(methanesulfonyl-phenyl- amino)-(2-oxo-pyrrolidin-1-yl)- benzamide (E44) | 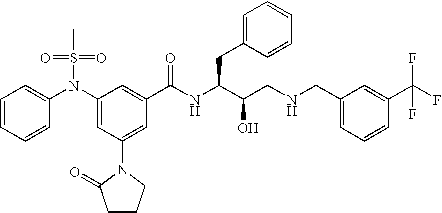 | A51 | B6 | 637.2 | 2.66 |
| N-[(1S,2R)-1-Benzyl-2-hydroxy-3- (3-trifluoromethyl-benzylamino)- propyl]-bromo-(methanesulfonyl- phenyl-amino)-benzamide (E45) | 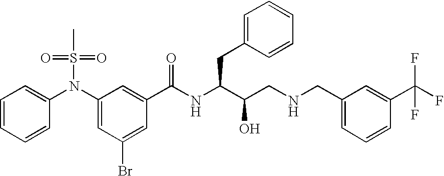 | A44 | B6 | 692.2 | 2.96 |
| N-[(1S,2R)-1-Benzyl-2-hydroxy-3- (3-trifluoromethyl-benzylamino)- propyl]-ethynyl-(methanesulfonyl- phenyl-amino)-benzamide (E46) | 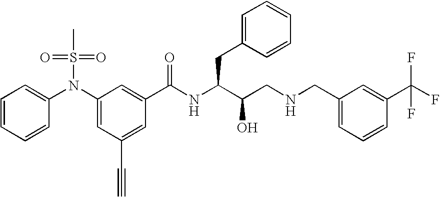 | A53 | B6 | 636.2 | 2.91 |
| (Methanesulfonyl-phenyl-amino)- methyl-biphenyl-3-carboxylic acid [(1S,2R)-1-benzyl-2-hydroxy-3- (3-trifluoromethyl-benzylamino)- propyl]-amide (E47) | 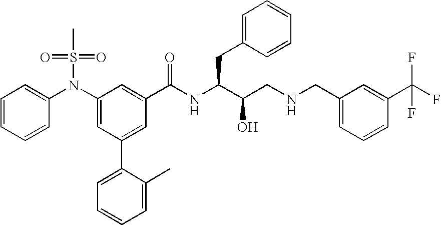 | A54 | B6 | 702.2 | 3.13 |
| (Methanesulfonyl-phenyl-amino)- dimethyl-biphenyl-3-carboxylic acid [(1S,2R)-1-benzyl-2-hydroxy-3- (3-trifluoromethyl-benzylamino)- propyl]-amide (E48) | 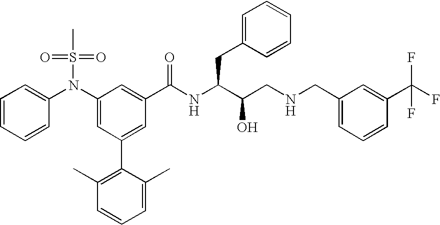 | A55 | B6 | 716.3 | 3.18 |
| N-[(1S,2R)-1-Benzyl-2-hydroxy-3- (3-trifluoromethyl-benzylamino)- propyl]-cyclopentyl- (methanesulfonyl-phenyl-amino)- benzamide (E49) | 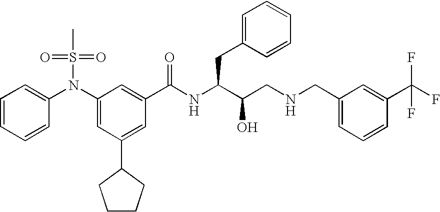 | A56 | B6 | 680.3 | 3.13 |
| N-[(1S,2R)-1-Benzyl-2-hydroxy-3- (3-trifluoromethyl-benzylamino)- propyl]-cyclohexyl- (methanesulfonyl-phenyl-amino)- benzamide (E50) | 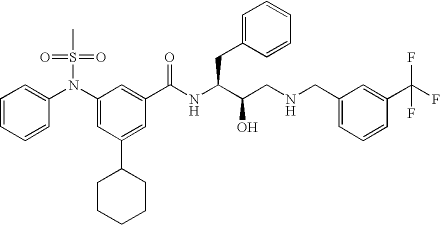 | A57 | B6 | 612.2 | 3.19 |
| N-[(1S,2R)-1-Benzyl-2-hydroxy-3- (3-trifluoromethyl-benzylamino)- propyl]-(methanesulfonyl-phenyl- amino)-prop-1-ynyl-benzamide (E51) | 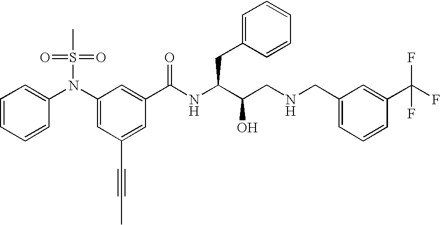 | A58 | B6 | 650.2 | 2.97 |
| N-[(1S,2R)-1-Benzyl-2-hydroxy-3- (3-trifluoromethyl-benzylamino)- propyl]-(methanesulfonyl-phenyl- amino)-nicotinamide (E52) | 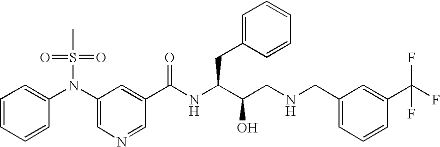 | A16 | B6 | 613.2 | 2.69 |
| N-[(1S,2R)-1-Benzyl-2-hydroxy-3- (3-trifluoromethyl-benzylamino)- propyl]-#Cl-(methanesulfonyl- phenyl-amino)-isonicotinamide (E53) | 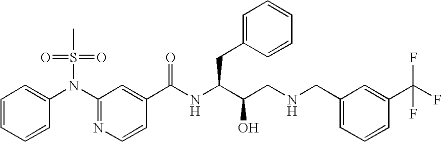 | A17 | B6 | 613.2 | 2.75 |
| N-[(1S,2R)-1-Benzyl-3- cyclopropylamino-2-hydroxy- propyl)-(methanesulfonyl- phenyl-amino)-(2-oxo-pyrrolidin- 1-yl)-benzamide (E54) | 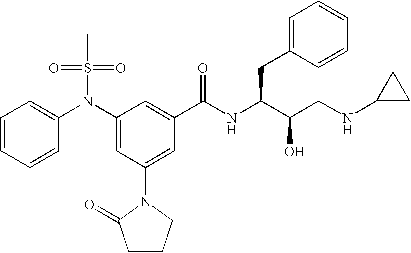 | A51 | B8 | 494.1 | 2.47 |
| N-[(1S,2R)-1-Benzyl-2-hydroxy-3- (3-trifluoromethoxy-benzylamino)- propyl]-(methanesulfonyl-phenyl- amino)-(2-oxo-pyrrolidin-1-yl)- benzamide formate salt (E55) | 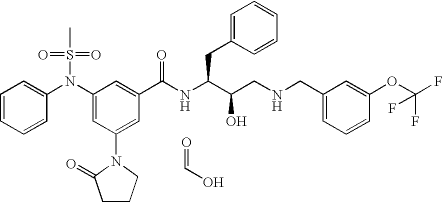 | A51 | B5 | 711.2 | 2.91 |
| N-[(1S,2R)-1-Benzyl-2-hydroxy-3- (3-trifluoromethoxy-benzylamino)- propyl]-C-fluoro-(methanesulfonyl- phenyl-amino)-(2-oxo-pyrrolidin-1- yl)-benzamide formate salt (E56) | 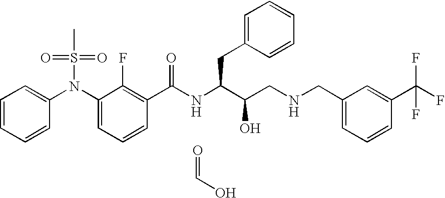 | A31 | B6 | 630.1 | 2.82 |
| N-[(1S,2R)-1-Benzyl-2-hydroxy-3- (3-trifluoromethoxy-benzylamino)- propyl]-fluoro-(methanesulfonyl- phenyl-amino)-benzamide formate salt (E57) | 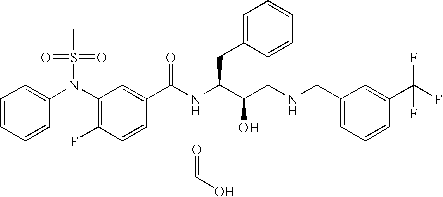 | A32 | B6 | 630.1 | 2.88 |
| N-[(1S,2R)-1-Benzyl-2-hydroxy-3- (3-trifluoromethoxy-benzylamino)- propyl]-(methanesulfanyl-phenyl- amino)-methylsulfanyl-benzamide formate salt (E58) | 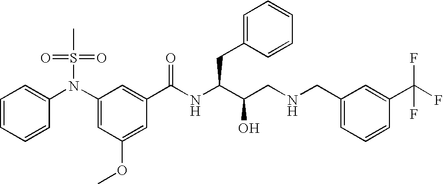 | A14 | B6 | 658.2 | 3.01 |
| N-[(1S,2R)-1-Benzyl-2-hydroxy-3- (3-trifluoromethoxy-benzylamino)- propyl]-methanesulfonyl- (methanesulfonyl-phenyl-amino)- benzamide (E59) | 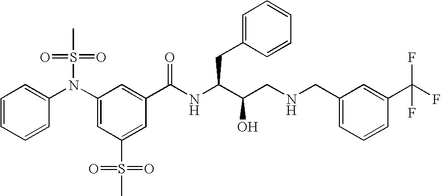 | A59 | B6 | 690.2 | 2.81 |
| N-[(1S,2R)-1-Benzyl-2-hydroxy-3- (3-trifluoromethoxy-benzylamino)- propyl]-ethylsulfanyl- (methanesulfonyl-phenyl-amino)- benzamide (E60) | 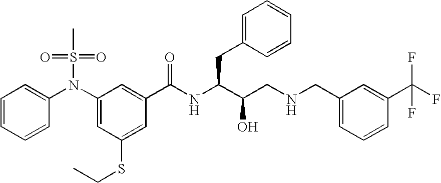 | A15 | B6 | 672.1 | 3.06 |
| N-[(1S,2R)-1-Benzyl-2-hydroxy-3- (3-trifluoromethoxy-benzylamino)- propyl]-ethanesulfonyl- (methanesulfonyl-phenyl-amino)- benzamide (E61) | 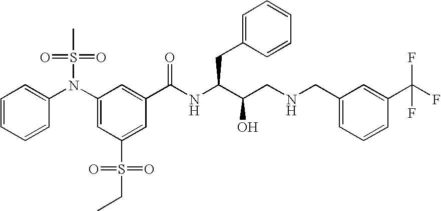 | A60 | B6 | 704.2 | 2.86 |
| N-[(1S,2R)-1-Benzyl-3-((S)-1- cyclohexylcarbamoyl-ethylamino)-2- hydroxy-proypl]-(1,1-dioxo-1I⁶- isothiazolidin-2-yl)-methanesulfonyl- phenyl-amino)-benzamide (E62) | 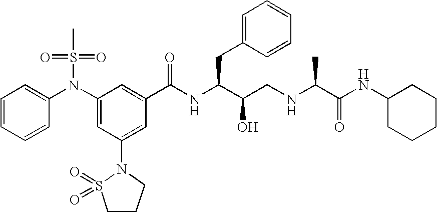 | A2 | B2 | 726.3 | 2.60 |
| N-[(1S,2R)-1-Benzyl-3-((S)-1- cyclohexylcarbamoyl-ethylamino)-2- hydroxy-propyl]-(methanesulfonyl- phenyl-amino)-N′-N′-dipropyl- isophthalamide (E63) | 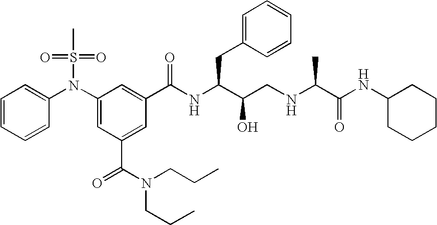 | A3 | B2 | 734.3 | 2.85 |
| N-[(1S,2R)-1-Benzyl-2-hydroxy-3- (3-trifluoromethyl-benzylamino)- propyl]-ethoxy-(methanesulfonyl- phenyl-amino)-benzamide (E64) | 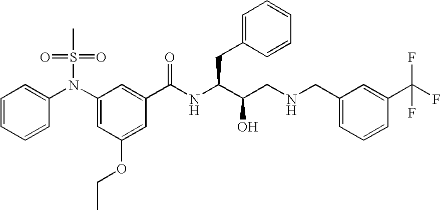 | A4 | B6 | 656.3 | 2.70 |
| N-[(1S,2R)-1-Benzyl-2-hydroxy-3- (3-trifluoromethyl-benzylamino)- propyl]-cyano-(methanesulfonyl- phenyl-amino)-benzamide (E65) | 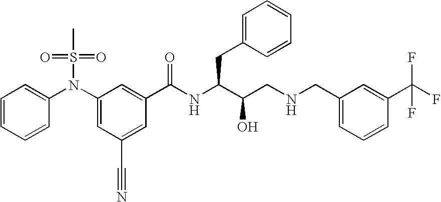 | A7 | B6 | 637.1 | 2.86 |
| N-[(1S,2R)-1-Benzyl-2-hydroxy-3- (3-trifluoromethyl-benzylamino)- propyl]-(methanesulfonyl-phenyl- amino)-C-methyl-benzamide (E66) | 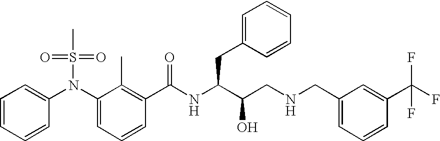 | A10 | B6 | 626.2 | 2.80 |
| N-[(1S,2R)-1-Benzyl-2-hydroxy-3- (3-trifluoromethyl-benzylamino)- propyl]-(methanesulfonyl-phenyl- amino)-methyl-benzamide (E67) | 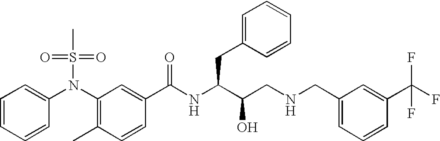 | A11 | B6 | 626.3 | 2.91 |
| N-[(1S,2R)-1-Benzyl-2-hydroxy-3- (3-trifluoromethyl-benzylamino)- propyl]-chloro-(methanesulfonyl- phenyl-amino)-benzamide (E68) | 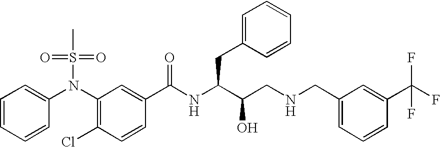 | A12 | B6 | 646.1 | 3.09 |
| N-[(1S,2R)-1-Benzyl-2-hydroxy-3- (3-trifluoromethyl-benzylamino)- propyl]-(methanesulfonyl-phenyl- amino)-C-methyl-benzamide (E69) | 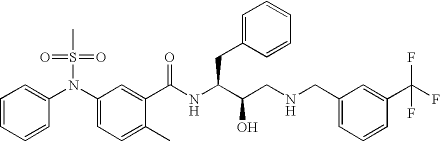 | A13 | B6 | 626.3 | 2.83 |
| N-[(1S,2R)-1-Benzyl-2-hydroxy-3- (3-trifluoromethyl-benzylamino)- propyl]-[(4-fluoro-phenyl)- methanesulfonyl-amino]-(2-oxo- pyrrolidin-1-yl)-benzamide formate salt (E70) | 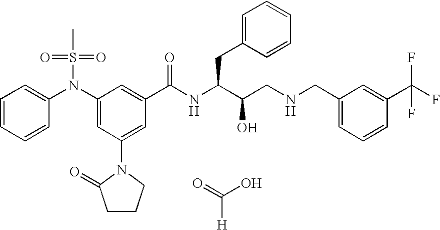 | A18 | B6 | 713.2 | 2.87 |
| N-[(1S,2R)-1-Benzyl-2-hydroxy-3- (3-trifluoromethyl-benzylamino)- propyl]-[(3-fluoro-phenyl)- methanesulfonyl-amino]-(2-oxo- pyrrolidin-1-yl)-benzamide formate salt (E71) | 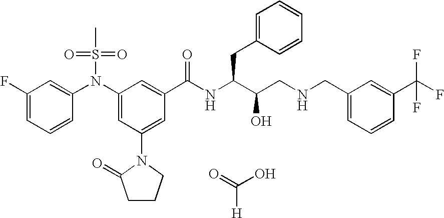 | A19 | B6 | 713.2 | 2.86 |
| N-[(1S,2R)-1-Benzyl-2-hydroxy-3- (3-trifluoromethyl-benzylamino)- propyl]-[(3-methoxy-phenyl)- methanesulfonyl-amino]-(2-oxo- pyrrolidin-1-yl)-benzamide formate salt (E72) | 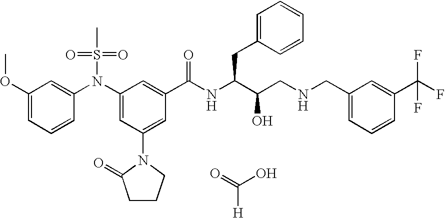 | A20 | B6 | 725.2 | 2.84 |
| N-[(1S,2R)-1-Benzyl-2-hydroxy-3- (3-trifluoromethyl-benzylamino)- propyl]-[(3,4-difluoro-phenyl)- methanesulfonyl-amino]-(2- oxo-pyrrolidin-1-yl)-benzamide formate salt (E73) | 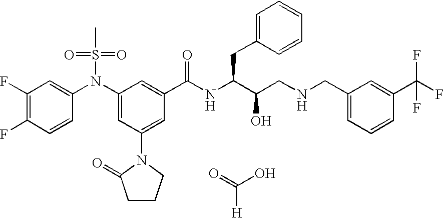 | A22 | B6 | 731.2 | 3.17 |
| N-[(1S,2R)-1-Benzyl-2-hydroxy-3- (3-trifluoromethyl-benzylamino)- propyl]-[methanesulfonyl-(4- methoxy-phenyl)-amino]-(2- oxo-pyrrolidin-1-yl)-benzamide formate salt (E74) | 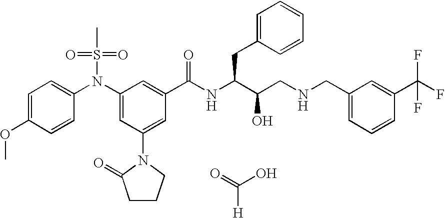 | A21 | B6 | 725.2 | 3.06 |
| N-[(1S,2R)-1-Benzyl-2-hydroxy-3- (3-trifluoromethyl-benzylamino)- propyl]-[(3,5-difluoro-phenyl)- methanesulfonyl-amino]-(2- oxo-pyrrolidin-1-yl)-benzamide formate salt (E75) | 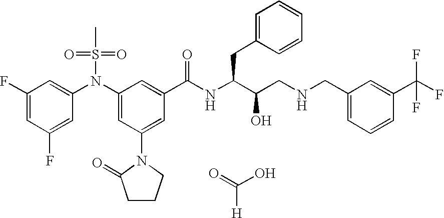 | A23 | B6 | 731.1 | 2.94 |
| N-[(1S,2R)-1-Benzyl-2-hydroxy-3- (3-trifluoromethyl-benzylamino)- propyl]-[(4-cyano-phenyl)- methanesulfonyl-amino]-(2- oxo-pyrrolidin-1-yl)-benzamide formate salt (E76) | 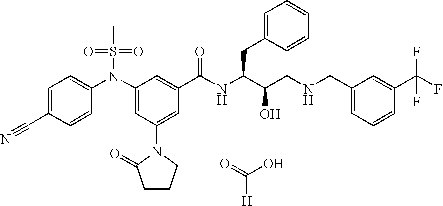 | A25 | B6 | 720.1 | 2.81 |
| N-[(1S,2R)-1-Benzyl-2-hydroxy-3- (3-trifluoromethyl-benzylamino)- propyl]-[(3-cyano-phenyl)- methanesulfonyl-amino]-(2- oxo-pyrrolidin-1-yl)-benzamide formate salt (E77) | 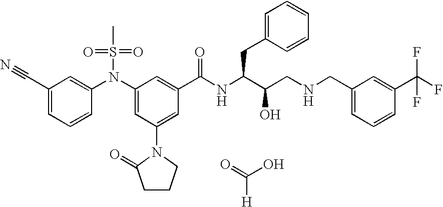 | A24 | B6 | 720.1 | 2.82 |
| N-[(1S,2R)-1-Benzyl-2-hydroxy-3- (3-trifluoromethyl-benzylamino)- propyl]-[(3-formamide-phenyl)- methanesulfonyl-amino]- (2-oxo-pyrrolidin-1-yl)-benzamide formate salt (E78) | 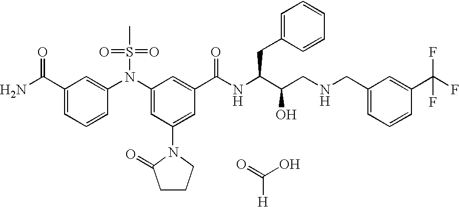 | A26 | B6 | 738.1 | 2.58 |
| N-[(1S,2R)-1-Benzyl-2-hydroxy-3- (3-trifluoromethyl-benzylamino)- propyl]-isobutyl-(methanesulfonyl- phenyl-amino)- benzamide (E79) | 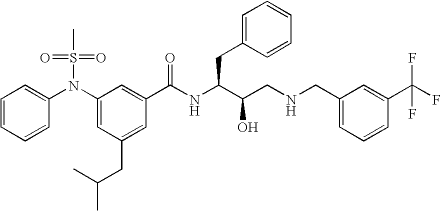 | A52 | B6 | 668.2 | 3.11 |

### Example 80

N-[(1S,2R)-Benzyl-hydroxy-(3-trifluoromethyl-benzylamino)-propyl]-3-(methanesulfonyl-phenyl-amino)-5-vinyl-benzamide (E80)

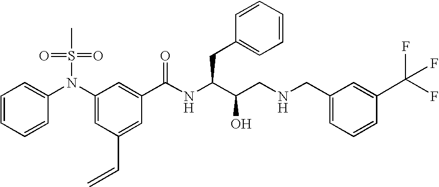

To a solution of N-[(1S,2R)-benzyl-hydroxy-(3-trifluoromethyl-benzylamino)-propyl]-3-bromo-5-(methanesulfonyl-phenyl-amino)-benzamide (E45) (140 mg, 0.2 mmol, 1 equiv) In DME (0.7 ml) and H₂O (0.6 ml) was added tetrakis(triphenylphosphine)-palladium(0) (12 mg, 0.01 mmol, 0.05 equiv), and the suspension was stirred for 10 min. 2,4,6 Trivinylcyclotriboroxane-pyridine complex (49 mg, 0.2 mmol, 1 equiv) in DME (1.3 ml) and K₂CO₃ (28 mg, 0.2 mmol, 1 equiv) were added and the resulting mixture was stirred at 90° C. for 1 h, cooled to room temperature and diluted with AcOEt. The organic phase was washed with H₂O, dried over MgSO₄ and concentrated in vacuo. Purification by flash chromatography on silica gel (CH₂Cl₂/MeOH: 96/4) gave N-[(1S,2R)benzyl-hydroxy-(3-trifluoromethyl-benzylamino)-propyl]-3-(methanesulfonyl-phenyl-amino-5-vinyl-benzamide (E80) (100 mg, 77%) as a pale yellow foam.

[M+H]⁺=638.2

RT=2.92 min

### Examples 81-83 (E81-E83)

Examples 81-83 were prepared in an analogous manner to Example 80 from Example 45 (E45) and the appropriate vinylcyclotriboroxane-pyridine complex (as described by F. Kerins and D. F. O'Shea in J. Org. Chem, 2002, 67, 4968-4971):

| | | Boroxane | | RT |
|---|---|---|---|---|
| Example | Structure | Complex | [M + H ]⁺ | (min) |
| N-[(1S,2R)-Benzyl- hydroxy-(3- trifluoromethyl- benzylamino)- propyl]- (methanesulfonyl- phenyl- amino)-(Z/E)- propenyl- benzamide (E81) | 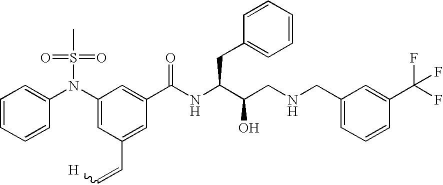 | 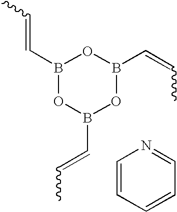 | 652.3 | 2.92 |
| N-[(1S,2R)-Benzyl- 2-hydroxy-3-(3- trifluoromethyl- benzylamino)- propyl]- 3-(methanesulfonyl- phenyl-amino)- 5-(2-methyl- propenyl)- benzamide (E82) | 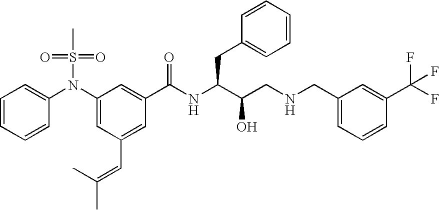 | 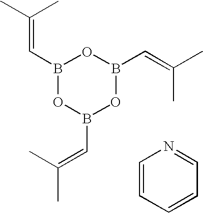 | 666.3 | 2.96 |
| N-[(1S,2R)-Benzyl- hydroxy-3-(3- trifluoromethyl- benzylamino)- propyl]- 3-isopropenyl-5- (methanesulfonyl- phenyl-amino)- benzamide (E83) | 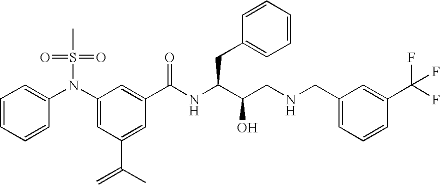 | 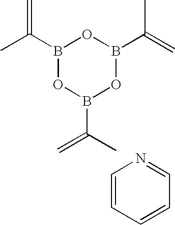 | 652.2 | 2.77 |

### Example 84

5-(Methanesulfonyl-phenyl-amino)-biphenyl-3-carboxylic acid [(1S,2R)-1-benzyl-2-hydroxy-3-(3-trifluoromethyl-benzylamino)-propyl]-amide (E84)

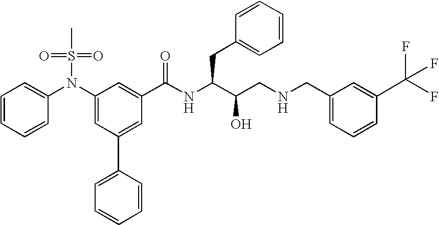

To a solution of N-[(1S,2R)-benzyl-hydroxy-(3-trifluoromethyl-benzylamino)-propyl]-3-bromo-5-(methanesulfonyl-phenyl-amino)-benzamide (E45) (140 mg, 0.2 mmol, 1 equiv) in toluene (10 ml) were added palladium(II)acetate (2 mg, 0.01 mmol, 0.05 equiv), phenylboronic acid (29 mg, 0.24 mmol, 1.2 equiv), 2-(dicyclohexylphosphino)biphenyl (7 mg, 0.02 mmol, 0.1 equiv) and K₃PO₄ (127 mg, 0.6 mmol, 3 equiv) and the resulting mixture was stirred at 100° C. for 16 h, cooled to room temperature and diluted with AcOEt. The organic phase was washed with water and brine, dried over MgSO₄ and concentrated in vacuo. Purification by flash chromatography on silica gel gave 5-(methanesulfonyl-phenyl-amino)biphenyl-3-carboxylic acid [(1S,2R)-1-benzyl-2-hydroxy-3-(3-trifluoromethyl-benzylamino)propyl]-amide (E84) (84 mg, 61%) as a pale yellow oil.

[M+H]⁺=688.2

RT=3.10 min

### Example 85

N-[(1S,2R)-Benzyl-hydroxy-(3-trifluoromethyl-benzylamino)-propyl]-3-ethyl-5-(methanesulfonyl-phenyl-amino)-benzamide (E85)

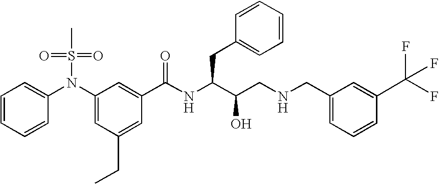

A flask was charged with N-[(1S,2R)-benzyl-hydroxy-(3-trifluoromethyl-benzylamino) propyl]-3-(methanesulfonyl-phenyl-amino)-5-vinyl-benzamide (E80) (100 mg, 0.16 mmol, 1 equiv), 10% palladium on charcoal (50% wet, 100 mg, 50% w/w), NH₄COOH (99 mg, 1.6 mmol, 10 equiv) H₂O (2 ml) and EtOH (10 ml). The resulting mixture was stirred at 50° C. for 4 h, cooled to room temperature and the catalyst was filtered off through a pad of celite. Most of the EtOH was removed in vacuo and the residue diluted with H₂O and AcOEt. The layers were separated. The organic phase was dried over MgSO₄ and concentrated in vacuo to give N-[(1S,2R)-benzyl-hydroxy-(3-trifluoromethyl-benzylamino)-propyl]-3-ethyl-5-(methanesulfonyl-phenyl-amino)-benzamide (E85) (70 mg, 70%) as a white solid.

[M+H]⁺=640.3

RT=2.86 min

### Examples 86-87 (E86-E87)

Examples 86-87 were prepared in an analogous manner to that described for Example 85 (E85) from the appropriate starting material:

| | | Starting | | RT |
|---|---|---|---|---|
| Example | Structure | Material | [M + H]⁺ | (min) |
| N-[(1S,2R)-Benzyl- hydroxy-(3- trifluoromethyl- benzylamino)-propyl]-3- (methanesulfonyl- phenyl-amino)-5-propyl- benzamide (E86) | 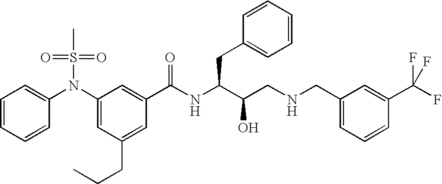 | E81 | 654.2 | 2.81 |
| N-[(1S,2R)-1-Benzyl-2- hydroxy-3-(3- trifluoromethyl- benzylamino)-propyl]-3- isopropyl-5- (methanesulfonyl- phenyl-amino)- benzamide (E87) | 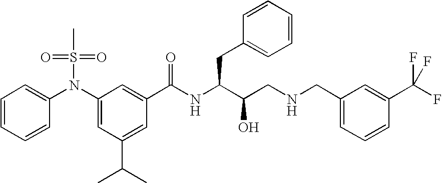 | E83 | 654.2 | 2.78 |

### Example 88

N-[(1S,2R)-1-Benzyl-3-((S)-1-cyclohexylcarbamoyl-ethylamino)-2-hydroxy-propyl]-3-(methanesulfonyl-phenyl-amino)-5-methylamino-benzamide (EBB)

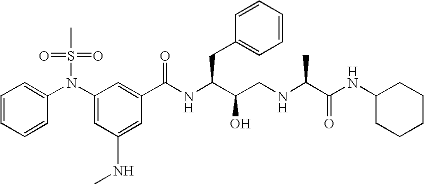

A flask was charged with N-[(1S,2R)-benzyl-((S)-1-cyclohexylcarbamoyl-ethylamino) hydroxy-propyl]-(benzyl-methyl-amino-(methanesulfonyl-phenyl-amino)-benzamide (E39) (50 mg, 0.07 mmol, 1 equiv), 10% palladium on charcoal (50% wet, 5 mg, 5% w/w), NH₄COOH (44 mg, 0.7 mmol, 10 equiv), H₂O (2 ml) and EtOH (10 ml). The resulting mixture was stirred at 50° C. for 3 h, cooled to room temperature and the catalyst was filtered off through a pad of celite. Most of the EtOH was removed in vacuo and the residue diluted with saturated NaHCO₃ aqueous solution and AcOEt. The layers were separated. The organic phase was dried over MgSO₄ and concentrated in vacuo. The residue was triturated with Et₂O to give N-[(1S,2R)-1-benzyl-3-((S)-1-cyclohexylcarbamoyl-ethylamino)-2-hydroxy-propyl]-3-(methanesulfonyl-phenyl-amino)-5-methylamino-benzamide (E88) (18 mg, 41%) as a white solid.

[M+H]⁺=636.2, RT=2.62 min

### Examples 89-90 (E89-E90)

Examples 89-90 were prepared in an analogous manner to Example 1 from the appropriate acid and amines indicated in the below table:

| | | | | | RT |
|---|---|---|---|---|---|
| Example | Structure | Acid | Amine | [M + H]⁺ | (min) |
| Ethyl-(methanesulfonyl- phenyl-amino)-1H- indole-5-carboxylic acid [(1S,2R)-1-benzyl-2- hydroxy-3-(3- trifluoromethyl- benzylamino)-propyl]- amide formate salt (E89) | 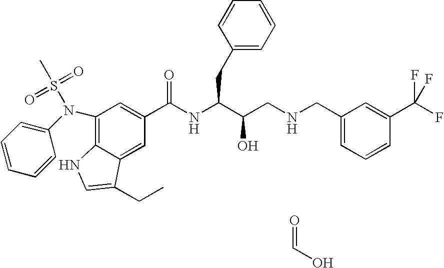 | A61 | B6 | 679.5 | 2.88 |
| Ethyl-(methanesulfonyl- phenyl-amino)-1H- indole-5-carboxylic acid [(1S,2R)-1-benzyl-2- hydroxy-3-(trimethyl- hexylamino)-propyl]- amide formate salt E90 | 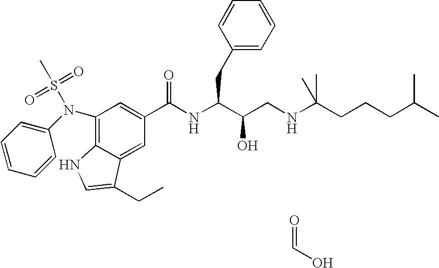 | A61 | B10 | 647.6 | 2.93 |

Compounds of the invention may be tested for in vitro biological activity in accordance with the following assays:

(I) Asp-2 Inhibitory Assay

For each compound being assayed, in a 384 well plate, is added:—

a) 1 μl of a DMSO solution of the test compound (IC₅₀ curve uses ten 1 in 2 serial dilutions from 500 μM).

b) 10 μl of substrate (FAM-[SEVNLDAEFK]-TAMRA) solution in buffer. This is prepared by diluting 2 ml of a 2 mM DMSO solution of the substrate into 400 ml of buffer (100 mM Sodium acetate pH=4.5, 1 l Milli-Q water, 0.06% Triton X-100 (0.5 ml/l), pH adjusted to 4.5 using glacial acetic acid). Aminomethyl fluorescein (FAM) and tetramethyl rhodamine (TAMRA) are fluorescent molecules which co-operate to emit fluorescence at 535 nm upon cleavage of the SEVNLDAEFK peptide.

c) 10 μl enzyme solution. This is prepared by diluting 16 ml of a 500 nM enzyme solution into 384 ml of buffer (prepared as above).

Blank wells (enzyme solution replaced by buffer) are included as controls on each plate. Wells are incubated for 1 h at room temperature and fluorescence read using a Tecan Ultra Fluorimeter/Spectrophotometer (485 nm excitation, 535 nm emission).

(II) Cathepsin D Inhibitory Assay

For each compound being assayed, in a 384 well plate, is added:—

a) 1 μl of a DMSO solution of the test compound (IC₅₀ curve uses ten 1 in 2 serial dilutions from 500 μM).

b) 10 μl of substrate (FAM-[SEVNLDAEFK]-TAMRA) solution in buffer. This is prepared by diluting 2 ml of a 2 mM DMSO solution of the substrate into 400 ml of buffer (100 mM Sodium acetate pH=4.5, 1 l Milli-Q water, 0.06% Triton X-100 (0.5 ml/l), pH adjusted to 4.5 using glacial acetic add).

c) 10 μl enzyme solution. This is prepared by diluting 1.6 ml of a 200 unit/ml (in 10 mM HCl) enzyme solution into 398.4 ml of buffer (prepared as above).

Blank wells (enzyme solution replaced by buffer) are included as controls on each plate. Wells are incubated for 1 h at room temperature and fluorescence read using a Tecan Ultra Fluorimeter/Spectrophotometer (485 nm excitation, 535 nm emission).

Pharmacological Data

The compounds of E1-E90 were tested in Assays (I) and (II) and exhibited inhibition within the following range: 1-5000 nM (Asp2) and 5025000 nM (CatD). More particularly, the compounds of E17, 25, 38, 41, 46, 49, 50, 55-56, 60, 61, 64, 77, 83 and 87 exhibited inhibition within the following range: 1-100 nM (Asp-2) and 200-2500 nM (CatD). Most particularly, the compounds of E38, 41, 49, 55, 60, 64, 77 and 87 exhibited inhibition within the following range: 1-10 nM (Asp-2) and 400-1000 nM.

### ABBREVIATIONS

DMF dimethylformamide
DMSO dimethylsulfoxide
DMAP dimethylaminophenol
DABCO 1,4-diazabicyclo [2.2.2] octane
DME dimethyl ether
THF tetrahydrofuran
HOBT N-hydroxybenzotriazole
FAM carboxyfluorescein
TAMRA carboxytetramethylrhodamine
[ ] single amino acid letter code relating to peptide sequence

What is claimed is:

1.-8. (canceled)

9. A compound of formula (I): 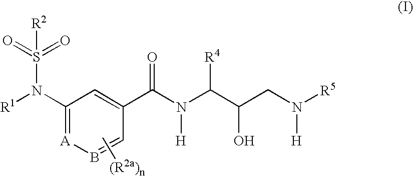
wherein
R¹ represents aryl or heteroaryl;
R² represents C₁₋₆ alkyl or C₃₋₈ cycloalkyl;
R^{2a} represents hydrogen, halogen, C₁₋₃ alkyl, or C₁₋₃ alkoxy;
n represents 0, 1, or 2;
A represents —C(H)═, —C(R^{2b})═, or —N═;
R^{2b} represents C₁₋₃ alkyl, C₂₋₄ alkenyl, halogen, C₁₋₃ alkoxy, amino, cyano, or hydroxy;
B represents —C(R³)═or —N═;
R³ represents hydrogen, halogen, optionally substituted C₁₋₆ alkyl, C₂₋₆ alkenyl, aryl, heteroaryl, heterocyclyl, —C₁₋₆ alkyl-aryl, —C₁₋₆ alkyl-heteroaryl, —C₁₋₆ alkyl-heterocyclyl, —C₂₋₆ alkenyl-aryl, —C₂₋₆ alkenyl-heteroaryl, —C₂₋₆ alkenyl-heterocyclyl, C₃₋₈ cycloalkyl, —C₁₋₆ alkyl-C₃₋₈ cycloalkyl, cyano, azido, nitro, sulphoxide, —NR⁷R⁸, —NR⁹COR¹⁰, —NR¹¹SO₂R¹², —NR¹¹CO₂R¹², —OR¹³, —SO₂R¹⁴, —SR¹⁵, —C≡CR¹⁶, —C₀₋₆ alkyl-(CF)_{q}CF_{3, —CONR}¹⁷R¹⁸, COOR¹⁹, —C₁₋₆ alkyl-NR²⁰R²¹, —C₁₋₆ alkyl-N₃, or R³ together with R^{2b} on adjacent carbon atoms may form a fused 5-7 membered saturated or partially saturated carbocyclic or heterocyclic ring optionally substituted by a C₁₋₆ alkyl group;
R⁴ represents optionally substituted C₁₋₆ alkyl, —C₁₋₆ alkyl-C₃₋₈ cycloalkyl, —C₁₋₆ alkyl-aryl, —C₁₋₆ alkyl-heteroaryl, or —C₁₋₆ alkyl-heterocyclyl;
R⁵ represents hydrogen, optionally substituted C₁₋₁₀ alkyl, —C₃₋₈ cycloalkyl, —C₃₋₈ cycloalkenyl, aryl, heteroaryl, heterocyclyl, —C₁₋₆ alkyl-C₃₋₈ cycloalkyl, —C₃₋₈ cycloalkyl-aryl, -heterocyclyl-aryl, —C₁₋₆ alkyl-aryl-heteroaryl, —C(R^{a}R^{b})—CONH—C₁₋₆ alkyl, —C(R^{c}R^{d})—CONH—C₃₋₈ cycloalkyl, —C₂₋₆ alkyl-S—C₁₋₆ alkyl, —C₂₋₆ alkyl-NR^{e}R^{f}, —C(R^{g}R^{h})—C₁₋₆ alkyl, —C(RⁱR^{j})-aryl, —C(R^{k}R^{l})—C₁₋₆ alkyl-aryl, —C(R^{m}Rⁿ)—C₁₋₆ alkyl-heteroaryl, —C(R^{o}R^{p})—C₁₋₆ alkyl-heterocyclyl, —C₁₋₆ alkyl-O—C₁₋₆ alkyl-aryl, —C₁₋₆ alkyl-O—C₁₋₆ alkyl-heteroaryl, or —C₁₋₆ alkyl-O—C₁₋₆ alkyl-heterocyclyl;
R⁷, R⁸, R⁹, R¹⁰, R¹¹, R¹³, R¹⁴, R¹⁵, R¹⁶, R¹⁷, R¹⁸, R¹⁹, R²⁰, and R²¹ independently represent hydrogen, C₁₋₆ alkyl, C₂₋₆ alkenyl, C₃₋₈ cycloalkyl, —CO—C₁₋₆ alkyl, aryl, heteroaryl, heterocyclyl, —C₁₋₆ alkyl-C₃₋₈ cycloalkyl, —C₁₋₆ alkyl-aryl, —C₁₋₆ alkyl-heteroaryl, or —C₁₋₆ alkyl-heterocyclyl;
R^{a}, R^{c}, R^{e}, R^{f}, R^{g}, R^{h}, Rⁱ, R^{j}, R^{k}, R^{l}, R^{m}, Rⁿ, R^{o} and R^{p} independently represent hydrogen, C₁₋₆ alkyl, or C₃₋₈ cycloalkyl;
R^{b} and R^{d} independently represent hydrogen, C₁₋₆ alkyl, C₃₋₈ cycloalkyl, —C₁₋₆ alkyl-SO₂—C₁₋₆ alkyl, or each combination of R^{a} and R^{b}, R^{c} and R^{d}, R^{g} and R^{h}, Rⁱ and R^{j}, R^{k} and R^{l} and R^{m} and Rⁿ together with the carbon atom to which they are attached may form a C₃₋₈ cycloalkyl group;
R¹² represents C₁₋₆ alkyl or C₃₋₈ cycloalkyl;
q represents 0 to 3;
optional substituents for alkyl groups of R³, R⁴, and R⁵ include one or more halogen, C₁₋₆ alkoxy, amino, cyano, or hydroxy groups;
and wherein said aryl, heteroaryl or heterocyclyl groups may be optionally substituted by one or more (i) C₁₋₆ alkyl, (ii) halogen, (iii) —CF₃, (iv) —OCF₃, ═O, (v) hydroxyl, (vi) C₁₋₆ alkoxy, (vii) C₂₋₆ alkynyl, (viii) C₂₋₆ alkenyl, (ix) amino, (x) cyano, (xi) nitro, (xii) —NR²²COR²³, (xiii) —CONR²²R²³—C₁₋₆ alkyl-NR²²R²³ in which R²² and R²³ independently represent hydrogen or C₁₋₆ alkyl, (xiv) —C₁₋₆ alkyl-O—C₁₋₆ alkyl, or (xv) —C₁₋₆ alkanoyl groups;
or a pharmaceutically acceptable salt or solvate thereof.

10. A compound according to claim 9 which is selected from the group consisting of:
N-[(1S,2R)-1-Benzyl-3-((S)-1-cyclohexylcarbamoyl-ethylamino)-2-hydroxy-propyl]-(methanesulfonyl-phenyl-amino)-benzamide;
N-[(1S,2R)-1-Benzyl-3-((S)-1-cyclohexylcarbamoyl-ethylamino)-2-hydroxy-propyl]-(ethanesulfonyl-phenyl-amino)-benzamide;
N-[(1S,2R)-1-Benzyl-3-((S)-1-cyclohexylcarbamoyl-ethylamino)-2-hydroxy-propyl]-[(butane-1-sulfonyl)-phenyl-amino]-benzamide;
N-[(1S,2R)-1-Benzyl-3-((S)-1-cyclohexylcarbamoyl-ethylamino)-2-hydroxy-propyl]-[phenyl-(propane-2-sulfonyl)-amino]-benzamide;
N-[(1S,2R)-1-Benzyl-3-((S)-1-cyclohexylcarbamoyl-ethylamino)-2-hydroxy-propyl]-[methanesulfonyl-(4-trifluoromethyl-phenyl)-amino]-benzamide;
N-[(1S,2R)-1-Benzyl-3-((S)-1-cyclohexylcarbamoyl-ethylamino)-2-hydroxy-propyl]-[methanesulfonyl-(3-methoxy-phenyl)-amino]-benzamide;
N-[(1S,2R)-1-Benzyl-3-((S)-1-cyclohexylcarbamoyl-ethylamino)-2-hydroxy-propyl]-[methanesulfonyl-(4-methoxy-phenyl)-amino]-benzamide;
N-[(1S,2R)-1-Benzyl-3-((S)-1-cyclohexylcarbamoyl-ethylamino)-2-hydroxy-propyl]-[methanesulfonyl-(2-methoxy-phenyl)-amino]-benzamide;
N-[(1S,2R)-1-Benzyl-3-((S)-1-cyclohexylcarbamoyl-ethylamino)-2-hydroxy-propyl]-[(3,4-dichloro-phenyl)-methanesulfonyl-amino]-benzamide;
N-[(1S,2R)-1-Benzyl-3-((S)-1-cyclohexylcarbamoyl-ethylamino)-2-hydroxy-propyl]-[(3,5-dichloro-phenyl)-methanesulfonyl-amino]-benzamide;
N-[(1S,2R)-1-Benzyl-3-((S)-1-cyclohexylcarbamoyl-ethylamino)-2-hydroxy-propyl]-[(2-cyano-phenyl)-methanesulfonyl-amino]-benzamide;
N-[(1S,2R)-1-Benzyl-3-((S)-1-cyclohexylcarbamoyl-ethylamino)-2-hydroxy-propyl]-[(4-cyano-phenyl)-methanesulfonyl-amino]-benzamide;
N-[(1S,2R)-1-Benzyl-3-((S)-1-cyclohexylcarbamoyl-ethylamino)-2-hydroxy-propyl]-[(2-chloro-phenyl)-methanesulfonyl-amino]-benzamide;
N-[(1S,2R)-1-Benzyl-3-((S)-1-cyclohexylcarbamoyl-ethylamino)-2-hydroxy-propyl]-[(4-chloro-phenyl)-methanesulfonyl-amino]-benzamide;
N-[(1S,2R)-1-Benzyl-3-((S)-1-cyclohexylcarbamoyl-ethylamino)-2-hydroxy-propyl]-[(3-cyano-phenyl)-methanesulfonyl-amino]-benzamide;
N-[(1S,2R)-1-Benzyl-3-((S)-1-cyclohexylcarbamoyl-ethylamino)-2-hydroxy-propyl]-[(3-chloro-phenyl)-methanesulfonyl-amino]-benzamide;
N-[(1S,2R)-1-Benzyl-2-hydroxy-3-(3-trifluoromethyl-benzylamino)-propyl]-[(3-cyano-phenyl)-methanesulfonyl-amino]-benzamide;
N-[(1S,2R)-1-Benzyl-2-hydroxy-3-(3-trifluoromethyl-benzylamino)-propyl]-[(3-chloro-phenyl)-methanesulfonyl-amino]-benzamide;
N-[(1S,2R)-1-Benzyl-2-hydroxy-3-(3-trifluoromethyl-benzylamino)-propyl]-[methanesulfonyl-(3-methoxy-phenyl)-amino]-benzamide;
N-[(1S,2R)-1-Benzyl-2-hydroxy-3-(3-trifluoromethyl-benzylamino)-propyl]-[methanesulfonyl-(4-methoxy-phenyl)-amino]-benzamide;
N-[(1S,2R)-1-Benzyl-2-hydroxy-3-(3-trifluoromethyl-benzylamino)-propyl]-[(4-chloro-phenyl)-methanesulfonyl-amino]-benzamide;
N-[(1S,2R)-1-Benzyl-2-hydroxy-3-(3-trifluoromethyl-benzylamino)-propyl]-[(3,5-dichloro-phenyl)-methanesulfonyl-amino]-benzamide;
N-[(1S,2R)-1-Benzyl-2-hydroxy-3-(3-trifluoromethyl-benzylamino)-propyl]-(methanesulfonyl-pyridin-3-yl-amino)-benzamide;
N-[(1S,2R)-1-Benzyl-2-hydroxy-3-(3-trifluoromethyl-benzylamino)-propyl]-(methanesulfonyl-pyridin-2-yl-amino)-benzamide;
N-[(1S,2R)-1-Benzyl-2-hydroxy-3-(3-trifluoromethyl-benzylamino)-propyl]-(methanesulfonyl-pyridin-4-yl-amino)-benzamide;
N-[(1S,2R)-1-Benzyl-2-hydroxy-3-(3-trifluoromethyl-benzylamino)-propyl]-(methanesulfonyl-naphthalen-1-yl-amino)-benzamide;
N-[(1S,2R)-1-Benzyl-2-hydroxy-3-(3-trifluoromethyl-benzylamino)-propyl]-(methanesulfonyl-phenyl-amino)-benzamide;
N-((1S,2R)-1-Benzyl-3-cyclopropylamino-2-hydroxy-propyl)-(methanesulfonyl-phenyl-amino)-benzamide;
N-{(1S,2R)-1-Benzyl-2-hydroxy-3-[1-(3-methoxy-phenyl)-1-methyl-ethylamino]-propyl}-(methanesulfonyl-phenyl-amino)-benzamide;
N-[(1S,2R)-1-Benzyl-2-hydroxy-3-(3-trifluoromethyl-benzylamino)-propyl]-(methanesulfonyl-naphthalen-2-yl-amino)-benzamide;
N-[(1S,2R)-1-Benzyl-2-hydroxy-3-(3-trifluoromethyl-benzylamino)-propyl]-[(4-fluoro-phenyl)-methanesulfonyl-amino]-benzamide;
N-[(1S,2R)-1-Benzyl-2-hydroxy-3-(3-trifluoromethyl-benzylamino)-propyl]-[(3-fluoro-phenyl)-methanesulfonyl-amino]-benzamide;
N-[(1S,2R)-1-Benzyl-2-hydroxy-3-(3-trifluoromethyl-benzylamino)-propyl]-[(3,5-difluoro-phenyl)-methanesulfonyl-amino]-benzamide;
N-[(1S,2R)-1-Benzyl-3-(1,5-dimethyl-hexylamino)-2-hydroxy-propyl]-(methanesulfonyl-phenyl-amino)-benzamide;
N-[(1S,2R)-1-Benzyl-2-hydroxy-3-(3-methoxy-benzylamino)-propyl]-(methanesulfonyl-phenyl-amino)-benzamide;
N-{(1S,2R)-1-Benzyl-3-[(1-ethyl-1H-pyrazol-4-ylmethyl)-amino]-2-hydroxy-propyl}-(methanesulfonyl-phenyl-amino)-benzamide;
N-((1S,2R)-1-Benzyl-3-cyclohexylamino-2-hydroxy-propyl)-(methanesulfonyl-phenyl-amino)-benzamide;
N-[(1S,2R)-1-Benzyl-3-((S)-1-cyclohexylcarbamoyl-ethylamino)-2-hydroxy-propyl]-ethylamino-(methanesulfonyl-phenyl-amino)-benzamide;
N-[(1S,2R)-Benzyl-((S)-1-cyclohexylcarbamoyl-ethylamino)-hydroxy-propyl]-(benzyl-methyl-amino)-(methanesulfonyl-phenyl-amino)-benzamide;
N-((1S,2R)-1-Benzyl-3-cyclohexylamino-2-hydroxy-propyl)-(methanesulfonyl-phenyl-amino)-(2-oxo-pyrrolidin-1-yl)-benzamide;
N-[(1S,2R)-1-Benzyl-3-((S)-1-cyclohexylcarbamoyl-ethylamino)-2-hydroxy-propyl]-(methanesulfonyl-phenyl-amino)-(2-oxo-pyrrolidin-1-yl)-benzamide;
N-[(1S,2R)-1-Benzyl-2-hydroxy-3-(3-methoxy-benzylamino)-propyl]-(methanesulfonyl-phenyl-amino)-(2-oxo-pyrrolidin-1-yl)-benzamide;
N-[(1S,2R)-1-Benzyl-3-(1,5-dimethyl-hexylamino)-2-hydroxy-propyl]-(methanesulfonyl-phenyl-amino)-(2-oxo-pyrrolidin-1-yl)-benzamide;
N-[(1S,2R)-1-Benzyl-2-hydroxy-3-(3-trifluoromethyl-benzylamino)-propyl]-(methanesulfonyl-phenyl-amino)-(2-oxo-pyrrolidin-1-yl)-benzamide;
N-[(1S,2R)-1-Benzyl-2-hydroxy-3-(3-trifluoromethyl-benzylamino)-propyl]-bromo-(methanesulfonyl-phenyl-amino)-benzamide;
N-[(1S,2R)-1-Benzyl-2-hydroxy-3-(3-trifluoromethyl-benzylamino)-propyl]-ethynyl-(methanesulfonyl-phenyl-amino)-benzamide;
(Methanesulfonyl-phenyl-amino)-methyl-biphenyl-3-carboxylic acid [(1S,2R)-1-benzyl-2-hydroxy-3-(3-trifluoromethyl-benzylamino)-propyl]-amide;
(Methanesulfonyl-phenyl-amino)-dimethyl-biphenyl-3-carboxylic acid [(1S,2R)-1-benzyl-2-hydroxy-3-(3-trifluoromethyl-benzylamino)-propyl]-amide;
N-[(1S,2R)-1-Benzyl-2-hydroxy-3-(3-trifluoromethyl-benzylamino)-propyl]-cyclopentyl-(methanesulfonyl-phenyl-amino)-benzamide;
N-[(1S,2R)-1-Benzyl-2-hydroxy-3-(3-trifluoromethyl-benzylamino)-propyl]-cyclohexyl-(methanesulfonyl-phenyl-amino)-benzamide;
N-[(1S,2R)-1-Benzyl-2-hydroxy-3-(3-trifluoromethyl-benzylamino)-propyl]-(methanesulfonyl-phenyl-amino)-prop-1-ynyl-benzamide;
N-[(1S,2R)-1-Benzyl-2-hydroxy-3-(3-trifluoromethyl-benzylamino)-propyl]-(methanesulfonyl-phenyl-amino)-nicotinamide;
N-[(1S,2R)-1-Benzyl-2-hydroxy-3-(3-trifluoromethyl-benzylamino)-propyl]-(methanesulfonyl-phenyl-amino)-isonicotinamide;
N-((1S,2R)-1-Benzyl-3-cyclopropylamino-2-hydroxy-propyl)-(methanesulfonyl-phenyl-amino)-(2-oxo-pyrrolidin-1-yl)-benzamide;
N-[(1S,2R)-1-Benzyl-2-hydroxy-3-(3-trifluoromethoxy-benzylamino)-propyl]-(methanesulfonyl-phenyl-amino)-(2-oxo-pyrrolidin-1-yl)-benzamide;
N-[(1S,2R)-1-Benzyl-2-hydroxy-3-(3-trifluoromethyl-benzylamino)-propyl]-C-fluoro-(methanesulfonyl-phenyl-amino)-benzamide;
N-[(1S,2R)-1-Benzyl-2-hydroxy-3-(3-trifluoromethyl-benzylamino)-propyl]-fluoro-(methanesulfonyl-phenyl-amino)-benzamide;
N-[(1S,2R)-1-Benzyl-2-hydroxy-3-(3-trifluoromethyl-benzylamino)-propyl]-(methanesulfonyl-phenyl-amino)-methylsulfanyl-benzamide;
N-[(1S,2R)-1-Benzyl-2-hydroxy-3-(3-trifluoromethyl-benzylamino)-propyl]-methanesulfonyl-(methanesulfonyl-phenyl-amino)-benzamide;
N-[(1S,2R)-1-Benzyl-2-hydroxy-3-(3-trifluoromethyl-benzylamino)-propyl]-ethylsulfanyl-(methanesulfonyl-phenyl-amino)-benzamide;
N-[(1S,2R)-1-Benzyl-2-hydroxy-3-(3-trifluoromethyl-benzylamino)-propyl]-ethanesulfonyl-(methanesulfonyl-phenyl-amino)-benzamide;
N-[(1S,2R)-1-Benzyl-3-((S)-1-cyclohexylcarbamoyl-ethylamino)-2-hydroxy-propyl]-(1,1-dioxo-1/⁶-isothiazolidin-2-yl)-(methanesulfonyl-phenyl-amino)-benzamide;
N-[(1S,2R)-1-Benzyl-3-((S)-1-cyclohexylcarbamoyl-ethylamino)-2-hydroxy-propyl]-(methanesulfonyl-phenyl-amino)-N′—N′-dipropyl-isophthalamide;
N-[(1S,2R)-1-Benzyl-2-hydroxy-3-(3-trifluoromethyl-benzylamino)-propyl]-ethoxy-(methanesulfonyl-phenyl-amino)-benzamide;
N-[(1S,2R)-1-Benzyl-2-hydroxy-3-(3-trifluoromethyl-benzylamino)-propyl]-cyano-(methanesulfonyl-phenyl-amino)-benzamide;
N-[(1S,2R)-1-Benzyl-2-hydroxy-3-(3-trifluoromethyl-benzylamino)-propyl]-(methanesulfonyl-phenyl-amino)-C-methyl-benzamide;
N-[(1S,2R)-1-Benzyl-2-hydroxy-3-(3-trifluoromethyl-benzylamino)-propyl]-(methanesulfonyl-phenyl-amino)-methyl-benzamide;
N-[(1S,2R)-1-Benzyl-2-hydroxy-3-(3-trifluoromethyl-benzylamino)-propyl]-chloro-(methanesulfonyl-phenyl-amino)-benzamide;
N-[(1S,2R)-1-Benzyl-2-hydroxy-3-(3-trifluoromethyl-benzylamino)-propyl]-(methanesulfonyl-phenyl-amino)-C-methyl-benzamide;
N-[(1S,2R)-1-Benzyl-2-hydroxy-3-(3-trifluoromethyl-benzylamino)-propyl]-[(4-fluoro-phenyl)-methanesulfonyl-amino]-(2-oxo-pyrrolidin-1-yl)-benzamide;
N-[(1S,2R)-1-Benzyl-2-hydroxy-3-(3-trifluoromethyl-benzylamino)-propyl]-[(3-fluoro-phenyl)-methanesulfonyl-amino]-(2-oxo-pyrrolidin-1-yl)-benzamide;
N-[(1S,2R)-1-Benzyl-2-hydroxy-3-(3-trifluoromethyl-benzylamino)-propyl]-[(3-methoxy-phenyl)-methanesulfonyl-amino]-(2-oxo-pyrrolidin-1-yl)-benzamide;
N-[(1S,2R)-1-Benzyl-2-hydroxy-3-(3-trifluoromethyl-benzylamino)-propyl]-[(3,4-difluoro-phenyl)-methanesulfonyl-amino]-(2-oxo-pyrrolidin-1-yl)-benzamide;
N-[(1S,2R)-1-Benzyl-2-hydroxy-3-(3-trifluoromethyl-benzylamino)-propyl]-[methanesulfonyl-(4-methoxy-phenyl)-amino]-(2-oxo-pyrrolidin-1-yl)-benzamide;
N-[(1S,2R)-1-Benzyl-2-hydroxy-3-(3-trifluoromethyl-benzylamino)-propyl]-[(3,5-difluoro-phenyl)-methanesulfonyl-amino]-(2-oxo-pyrrolidin-1-yl)-benzamide;
N-[(1S,2R)-1-Benzyl-2-hydroxy-3-(3-trifluoromethyl-benzylamino)-propyl]-[(4-cyano-phenyl)-methanesulfonyl-amino]-(2-oxo-pyrrolidin-1-yl)-benzamide;
N-[(1S,2R)-1-Benzyl-2-hydroxy-3-(3-trifluoromethyl-benzylamino)-propyl]-[(3-cyano-phenyl)-methanesulfonyl-amino]-(2-oxo-pyrrolidin-1-yl)-benzamide;
N-[(1S,2R)-1-Benzyl-2-hydroxy-3-(3-trifluoromethyl-benzylamino)-propyl]-[(3-formamide-phenyl)-methanesulfonyl-amino]-(2-oxo-pyrrolidin-1-yl)-benzamide;
N-[(1S,2R)-1-Benzyl-2-hydroxy-3-(3-trifluoromethyl-benzylamino)-propyl]-isobutyl-(methanesulfonyl-phenyl-amino)-benzamide;
N-[(1S,2R)-Benzyl-hydroxy-(3-trifluoromethyl-benzylamino)-propyl]-3-(methanesulfonyl-phenyl-amino)-5-vinyl-benzamide;
N-[(1S,2R)-Benzyl-hydroxy-(3-trifluoromethyl-benzylamino)-propyl]-(methanesulfonyl-phenyl-amino)-(Z/E)-propenyl-benzamide;
N-[(1S,2R)-1-Benzyl-2-hydroxy-3-(3-trifluoromethyl-benzylamino)-propyl]-3-(methanesulfonyl-phenyl-amino)-5-(2-methyl-propenyl)-benzamide;
N-[(1S,2R)-1-Benzyl-2-hydroxy-3-(3-trifluoromethyl-benzylamino)-propyl]-3-isopropenyl-5-(methanesulfonyl-phenyl-amino)-benzamide;
5-(Methanesulfonyl-phenyl-amino)-biphenyl-3-carboxylic acid [(1S,2R)-1-benzyl-2-hydroxy-3-(3-trifluoromethyl-benzylamino)-propyl]-amide;
N-[(1S,2R)-Benzyl-hydroxy-(3-trifluoromethyl-benzylamino)-propyl]-3-ethyl-5-(methanesulfonyl-phenyl-amino)-benzamide;
N-[(1S,2R)-Benzyl-hydroxy-(3-trifluoromethyl-benzylamino)-propyl]-3-(methanesulfonyl-phenyl-amino)-5-propyl-benzamide;
N-[(1S,2R)-1-Benzyl-2-hydroxy-3-(3-trifluoromethyl-benzylamino)-propyl]-3-isopropyl-5-(methanesulfonyl-phenyl-amino)-benzamide;
N-[(1S,2R)-1-Benzyl-3-((S)-1-cyclohexylcarbamoyl-ethylamino)-2-hydroxy-propyl]-3-(methanesulfonyl-phenyl-amino)-5-methylamino-benzamide;
Ethyl-(methanesulfonyl-phenyl-amino)-1H-indole-5-carboxylic acid [(1S,2R)-1-benzyl-2-hydroxy-3-(3-trifluoromethyl-benzylamino)-propyl]-amide; and
Ethyl-(methanesulfonyl-phenyl-amino)-1H-indole-5-carboxylic acid [(1S,2R)-1-benzyl-2-hydroxy-3-(trimethyl-hexylamino)-propyl]-amide;
or a pharmaceutically acceptable salt or solvate thereof.

11. A pharmaceutical composition comprising a compound of claim 9 or a pharmaceutically acceptable salt or solvate thereof in admixture with one or more pharmaceutically acceptable diluents or carriers.

12. A pharmaceutical composition comprising a compound of claim 10 or a pharmaceutically acceptable salt or solvate thereof in admixture with one or more pharmaceutically acceptable diluents or carriers.

13. A method of treatment or prophylaxis of diseases characterised by elevated β-amyloid levels or β-amyloid deposits which comprises administering to a patient an effective amount of a compound of claim 9 or a pharmaceutically acceptable salt or solvate thereof.

14. A method of treatment of prophylaxis of diseases characterized by elevated β-amyloid levels or β-amyloid deposits which comprises administering to a patient an effective amount of a compound of claim 10 or a pharmaceutically acceptable salt or solvate thereof.

15. A pharmaceutical composition comprising a compound of claim 9 or a pharmaceutically acceptable salt or solvate thereof for use in the treatment of diseases characterised by elevated β-amyloid levels or β-amyloid deposits.

16. A pharmaceutical composition comprising a compound of claim 10 or a pharmaceutically acceptable salt or solvate thereof for use in the treatment of diseases characterised by elevated β-amyloid levels or β-amyloid deposits.

17. A pharmaceutical composition comprising a compound of formula (I) as defined in claim 9 or a pharmaceutically acceptable salt or solvate thereof in admixture with one or more pharmaceutically acceptable diluents or carriers.
